(19) United States
(12) Patent Application Publication
(10) Pub. No.: US 2022/0062281 A1
(43) Pub. Date: Mar. 3, 2022

(54) INHIBITORS TO TARGET HIV-1 NEF-CD80/CD86 INTERACTIONS FOR THERAPEUTIC INTERVENTION

(71) Applicants: NATIONAL CENTRE FOR BIOLOGICAL SCIENCES-TIFR, Karnataka Bengaluru (IN); CENTRE FOR CELLULAR AND MOLECULAR PLATFORMS NCBS-TIFR, Karnataka Bengaluru (IN); INDIAN INSTITUTE OF INTEGRATIVE MEDICINE, Jammu (IN); INTERNATIONAL CENTRE FOR GENETIC ENGINEERING AND BIOTECHNOLOGY, Delhi New Delhi (IN)

(72) Inventors: Satyajit MAYOR, Bangalore (IN); Akankshi MUNJAL, Bangalore (IN); Taslimarlf SAIYED, Karnataka Bangalore North (IN); Anandl KARUMBATl, Bangalore (IN); Ram Aysre VISHWAKARMA, Jammu (IN); Parvinder Pal SINGH, Jammu (IN); Shweta SHARMA, Jammu (IN); Gurunadham MUNAGALA, Jammu (IN); Kushalava Reddy YEMPALLA, Jammu (IN); Srinivas AMBALA, Jammu (IN); Shahid JAMEEL, New Delhi (IN); Satyajit RATH, Pune (IN)

(21) Appl. No.: 17/268,440

(22) PCT Filed: Aug. 13, 2019
(86) PCT No.: PCT/IN2019/050594

§ 371 Date: Feb. 12, 2021

(30) Foreign Application Priority Data
Aug. 13, 2018 (IN) 201841005491

(51) Int. Cl. A61K 31/506; A61K 31/5377; A61K 31/167; A61K 31/235; A61K 31/192; A61K 31/4245; A61K 31/421; A61K 31/27; A61P 31/18

(52) U.S. Cl. CPC A61K 31/506; A61K 31/5377; A61K 31/167; A61K 31/235; A61P 31/18; A61K 31/4245; A61K 31/421; A61K 31/27; A61K 31/192

(57) ABSTRACT

The compounds of Formula I, II, and III along with their stereoisomers, pharmaceutically acceptable salts, polymorphs, solvates and hydrates thereof are described in the present disclosure. The said compounds restore immune activation in case of infections or a disease associated with an HIV infection in a subject in need thereof.

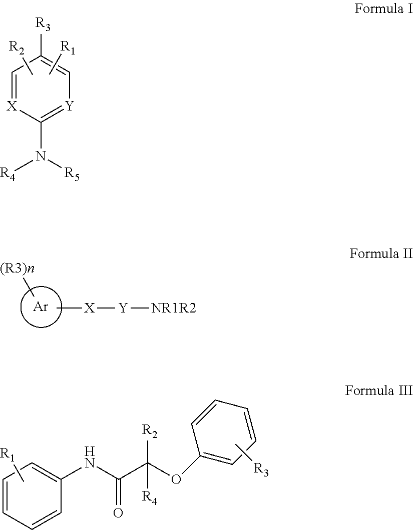

### FIELD OF INVENTION

The present disclosure relates to prevention/treatment of immune evasion by human immunodeficiency virus (HIV) related infections. The compounds of the present disclosure are useful as medicaments and their use in the manufacture of medicaments for treatment, prevention or suppression of diseases, and conditions mediated by HIV.

### BACKGROUND

In recent years, there has been considerable progress in the treatment of HIV-related illness through different approaches. One of them being use of highly active antiretroviral therapy (HAART), which involves the use of different kinds of anti-retroviral agents that act on different stages of the HIV life cycle. The anti-retro viral drugs have different mechanism of action, such as nucleoside/nucleotide reverse transcriptase inhibitors, non-nucleoside retroviral treatment, maturation and cellular inhibitors, integrase inhibitors, protease inhibitors and immune-based therapy. Despite the rapid development of the HIV therapy, the key issue for eradication of virus reservoir composed of latently infected memory CD4+ T cells carrying integrated pro-virus remains. The resting memory CD4+ T cells can persist for months to years carrying replication competent viral genome thus posing problems for patients on HAART. The current therapy does not eradicate the provirus and needs more research to address this issue.

Nef protein, a HIV-1 auxiliary protein, and inhibition of its interaction and complexes comprising Nef, with other cellular components have been studied in detail. Nef interaction with Src protein-tyrosine kinase family (series of signaling molecules) is required for the onset and progression of AIDS in HIV-1-infected persons. (Lugari et. al, Bioorganic & Medicinal Chemistry 19 (2011) 7401-7406; Emert-Sedlak et al., ACS Chem Biol. 2009 4(11), 939-947; U.S. Pat. No. 8,541,415).

Interestingly, the virus uses the Nef protein to evade killing of its host cell by cytotoxic T-lymphocytes. Inhibiting Nef-mediated functions thus presents a different strategy for increasing CTL activity against HIV infected cells by making the latter more visible to the immune system.

### SUMMARY

The present disclosure relates to compounds of Formula I, II, and III that restore immune activation in case of infections. The present disclosure specifically relates to a method for the prevention or treatment of an HIV infection or a disease associated with an HIV infection in a subject in need thereof, comprising: administering to a HIV infected subject a therapeutic dose of a compound selected from the group consisting of Formula I,

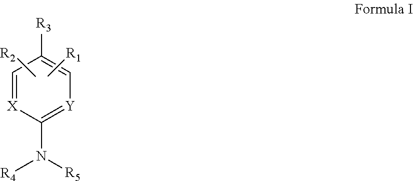

or its stereoisomers, pharmaceutically acceptable salts, polymorphs, solvates and hydrates thereof, wherein
R₁ and R₂ are independently selected from the group consisting of hydrogen, C₆₋₁₀ aryl, C₁₋₁₀ alkyl, C₂₋₁₀ alkenyl, C₂₋₁₀ alkynyl, and amino, wherein C₁₋₁₀ alkyl, C₂₋₁₀ alkenyl, C₂₋₁₀ alkynyl, and C₆₋₁₀ aryl are optionally substituted with one to four substituents independently selected from hydroxyl, halogen, C₁₋₁₀ alkyl, C₁₋₁₀ alkoxy, and C₂₋₁₀ heterocyclyl;
R₃ is C₆₋₁₀ aryl, wherein C₆₋₁₀ aryl is optionally substituted with one to four substituents independently selected from C₁₋₁₀ alkoxy, nitro, halogen or C₁₋₁₀ alkyl, wherein C₁₋₁₀ alkyl and C₁₋₁₀ alkoxy is optionally substituted with one to four substituents selected from halogen, hydrogen, hydroxyl, C₁₋₁₀ alkyl, C₁₋₁₀ alkoxy or C₂₋₁₀ heterocyclyl;
R₄ and R₅ are independently selected from the group consisting of hydrogen, C₁₋₁₀ alkyl, and —N═CHC₆ aryl, wherein N═CHC₆ aryl is further substituted with one to four halogen;
or R₄ and R₅ are joined to form a C₂₋₁₀ heterocyclyl or a C₂₋₁₀ heteroaryl, wherein C₂₋₁₀ heterocyclyl or C₂₋₁₀ heteroaryl, is optionally substituted with one to four substituents independently selected from C₁₋₁₀ alkyl; and
X and Y are N;

### Formula II,

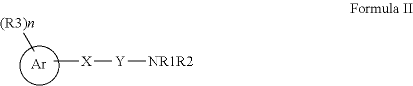 [0013] or its stereoisomers, pharmaceutically acceptable salts, polymorphs, solvates and hydrates thereof, wherein,
R₁, and R₂ are independently selected from the group consisting of hydrogen, C₁₋₁₀ alkyl, hydroxy, thio, amino, C₂₋₁₀ heterocyclyl, C₅₋₁₀ aryl, C₂₋₁₀ heteroaryl, acyl, amido, imido, sulfinyl, sulfonyl, carboxaldehyde, cyano, isocyano, azido, hydrazino, nitro, and halo; or
R₁ and R₂ are joined to form an optionally substituted monocyclic or a bicyclic ring, wherein C₁₋₁₀ alkyl, C₂₋₁₀ heterocyclyl, C₅₋₁₀ aryl, C₂₋₁₀ heteroaryl or monocyclic or a bicyclic ring are optionally substituted with one four substituents independently selected from hydroxy, C₁₋₁₀ alkyl, C₅₋₁₀ aryl, C₁₋₁₀ alkoxy, halogen, haloalkyl, perhaloalkyl, cyano, thio, and —CH₂-Ph-NH—COO-Ph-Me;
X is O;
Y is C═O;
Ar is selected from C₁₋₁₀ alkyl, C₅₋₁₀ aryl or C₂₋₁₀ heteroaryl;
R₃ is absent or is selected from hydroxy, C₁₋₁₀ alkyl, C₅₋₁₀ aryl, C₂₋₁₀ heteroaryl, C₂₋₁₀ heterocyclyl, C₁₋₁₀ alkoxy, halogen, haloalkyl, perhaloalkyl, nitro, cyano, CH₂PhNHCOOPh-Me, NHCONHPh-(Cl)₂ or CONHNHSO₂Ph-Me; and
n is selected from 0-5; and

### Formula III

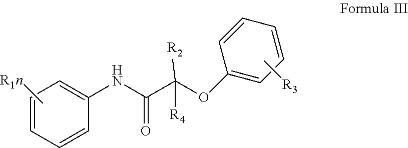 [0022] or its stereoisomers, pharmaceutically acceptable salts, polymorphs, solvates and hydrates thereof, wherein,
R₁ is independently selected from the group consisting of OH, COOH, and COORa, wherein Ra is C₁₋₁₀ alkyl;
R₂ and R₄ is independently selected from hydrogen or C₁₋₁₀ alkyl;
R₃ is selected from C₁₋₁₀ alkyl or halogen; and n is selected from 1 to 5.

The present disclosure further relates to a compound selected from the group consisting of Formula I, Formula II, and Formula III or its stereoisomers, pharmaceutically acceptable salts, complexes, hydrates, solvates, tautomers, polymorphs, racemic mixtures, optically active forms and pharmaceutically active derivative thereof, for prophylaxis and/or treatment of an HIV infection or a disease associated with an HIV infection

The present disclosure further relates to use of a compound selected from the group consisting of Formula I, Formula II, and Formula III or its stereoisomers, pharmaceutically acceptable salts, complexes, hydrates, solvates, tautomers, polymorphs, racemic mixtures, optically active forms and pharmaceutically active derivative thereof, towards restoration of immune signaling via T cell activation through inhibiting Nef-CD80/86 interactions.

The present disclosure further relates to a compound selected from the group consisting of Formula I, Formula II, and Formula III or its stereoisomers, pharmaceutically acceptable salts, complexes, hydrates, solvates, tautomers, polymorphs, racemic mixtures, optically active forms and pharmaceutically active derivative thereof, for use in treating a disease or condition in a patient wherein said disease or condition is caused by cancers including chronic lymphocytic leukemia, colon carcinoma, multiple myeloma or viral infections including HIV, HPV, herpes and the like.

The present disclosure further relates to use of a compound selected from the group consisting of Formula I, Formula II, and Formula III or its stereoisomers, pharmaceutically acceptable salts, complexes, hydrates, solvates, tautomers, polymorphs, racemic mixtures, optically active forms and pharmaceutically active derivative thereof, in treating disease or condition in a subject, wherein said disease or condition is caused by HIV. The subject may be a mammal including humans. These and other features, aspects, and advantages of the present subject matter will become better understood with reference to the following description. This summary is provided to introduce a selection of concepts in a simplified form. This summary is not intended to identify key features or essential features of the disclosure, nor is it intended to be used to limit the scope of the subject matter.

### BRIEF DESCRIPTION OF DRAWINGS

FIG. 1 illustrates the biochemical screening of the compounds in accordance with an implementation of the present disclosure.

FIG. 2 illustrates the schematic representation of the cell-based assay, in accordance with an implementation of the present disclosure.

FIG. 3 illustrates the inhibition of Nef mediated internalization of surface CD80/86 as measured by flow cytometry by compounds from Formula I, II and III in accordance with an implementation of the present disclosure.

FIG. 4 illustrates the schematic representation of the cell-based T cell activation assay in accordance with an implementation of the present disclosure.

FIG. 5 illustrates the inhibition of Nef mediated T cell inactivation in an APC in accordance with an implementation of the present disclosure.

### DETAILED DESCRIPTION

Those skilled in the art will be aware that the present disclosure is subject to variations and modifications other than those specifically described. It is to be understood that the present disclosure includes all such variations and modifications. The disclosure also includes all such steps, features, compositions and compounds referred to or indicated in this specification, individually or collectively, and any and all combinations of any or more of such steps or features.

### Definitions

For convenience, before further description of the present disclosure, certain terms employed in the specification, and examples are collected here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. Throughout this specification, unless the context requires otherwise the word “comprise”, and variations, such as “comprises” and “comprising”, will be understood to imply the inclusion of a stated element or step or group of element or steps but not the exclusion of any other element or step or group of element or steps.

The term “including” is used to mean “including but not limited to”. “Including” and “including but not limited to” are used interchangeably.

In the structural formulae given herein and throughout the present disclosure, the following terms have been indicated meaning, unless specifically stated otherwise.

The term “therapeutic dose” refers to providing any compound of the present disclosure (drug) in a dose per unit time over an extended time to a subject in need thereof. The therapeutic dose according to the present disclosure may be in a range of 50 μM to 1000 μM.

The term “relevant dose” refers to providing any compound of the present disclosure (drug) in a dose per unit time over an extended time to a subject for preventing HIV.

The term “low levels of CD80/86 receptors” can be defined as any condition which leads to a decrease in levels of CD80/86 receptors on the T cells.

The term “alkyl” refers to a monoradical branched or unbranched saturated hydrocarbon chain having from 1 to 10 carbon atoms. This term is exemplified by groups such as n-butyl, iso-butyl, t-butyl, n-hexyl, n-decyl, and the like. The groups may be optionally substituted.

The term “alkenyl” refers to a monoradical of a branched or unbranched unsaturated hydrocarbon group preferably having from 4, 5, 6, 7, 8, 9, or 10 carbon atoms and having 1, 2, 3, 4, 5 or 6 double bond (vinyl), preferably 1 double bond. The groups may be optionally substituted.

The term “alkynyl” refers to a monoradical of an unsaturated hydrocarbon, preferably having from 4, 5, 6, 7, 8, 9, or 10 carbon atoms and having 1, 2, 3, 4, 5 or 6 sites of acetylene (triple bond) unsaturation, preferably 1 triple bond. The groups may be optionally substituted.

“Halo” or “Halogen”, alone or in combination with any other term means halogens such as chloro (Cl), fluoro (F), bromo (Br) and iodo (I).

The term “aryl” refers to an aromatic carbocyclic group of 5 to 18 carbon atoms having a single ring (e.g. phenyl) or multiple rings (e.g. biphenyl), or multiple condensed (fused) rings (e.g. naphthyl or anthranyl). Preferred aryls include phenyl, naphthyl and the like. The groups may be optionally substituted.

The term “heteroaryl” refers to a heteroaromatic carbocyclic group of 2 to 10 carbon atoms having a single ring or multiple rings, or multiple condensed rings. Preferred heteroaryls include pyrazole, thiazole, oxazole, benzoxazole, pyridine and the like. The groups may be optionally substituted.

The term “cycloalkyl” refers to carbocyclic groups of from 3 to 12 carbon atoms having a single cyclic ring or multiple condensed rings, which may be partially unsaturated. Such cycloalkyl groups include, by way of example, single ring structures such as cyclopropyl, cyclohexyl, cyclohexenyl, and the like, or multiple ring structures or carbocyclic groups to which is fused an aryl group. The groups may be optionally substituted.

The term “heterocyclyl” refers to a saturated or partially unsaturated group or unsaturated group having a single ring or multiple condensed rings, having from 2 to 10 carbon atoms and from 1 to 10 hetero atoms, preferably 1, 2, or 3 heteroatoms, selected from nitrogen, sulfur, phosphorus, and/or oxygen within the ring. Preferred heterocyclyls include morpholine, piperidine, and the like. The groups may be optionally substituted.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds.

The term “effective amount” means an amount of a compound or composition, which is sufficient enough to significantly and positively modify the symptoms and/or conditions to be treated. The effective amount will vary depending on various conditions and is within the knowledge and expertise of the attending physician.

The compounds described herein may contain one or more chiral centers and/or double bonds and therefore, may exist as stereoisomers. Accordingly, the chemical structures depicted herein encompass all possible enantiomers and stereoisomers of the illustrated or identified compounds including the stereoisomerically pure form and enantiomeric and stereoisomeric mixtures. The compounds may also exist in several tautomeric forms including the enol form, the keto form and mixtures thereof. Accordingly, the chemical structures depicted herein encompass all possible tautomeric forms of the illustrated or identified compounds.

The term “pharmaceutically acceptable” refers to those compounds, materials, compositions, and/or dosage forms, which are suitable for use in contact with the tissues of human beings and animals and is understood by a person skilled in the art.

“Pharmaceutically acceptable salt” embraces salts with a pharmaceutically acceptable acid or base. Pharmaceutically acceptable acids include both inorganic acids, and organic bases.

The term “polymorphs” refers to crystal forms of the same molecule, and different polymorphs may have different physical properties.

The term “solvate”, as used herein, refers to a crystal form of a substance, which contains solvent.

The term “hydrate” refers to a solvate wherein the solvent is water. Ratios, concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a concentration range of about 50 to 1000 μM should be interpreted to include not only the explicitly recited limits of about 50 μM to about 1000 μM, but also to include sub-ranges, such as 75-875 μM, 80-500 μM, and so forth, as well as individual amounts, including fractional amounts, within the specified ranges, such as 100 μM, and 155 μM, for example.

The present disclosure relates to compounds for treatment of HIV infection. The target is novel interaction of Nef protein from the HIV-1 virus with the host immune receptors CD80/CD86 responsible for T cell stimulation.

An indispensable factor for HIV pathogenicity is the Nef protein. It allows HIV to evade the immune response, maintain high viral load and is needed for replication and dissemination, down-regulate cell surface receptors involved in the generation of immune response, including MHC-1 and MHC-2. It has been surprising found that in addition to relocation of MHC, Nef also down-regulates surface expression of the B-7 family of co-stimulatory proteins, namely CD80 and CD86 expressed on antigen presenting cells, leading to impaired T cell stimulation in vitro. Nef binds directly to the cytoplasmic tails of CD80 and CD86 and prone to reversal by introduction of peptides, making it a suitable target for therapeutic intervention. Targeting Nef-CD80/86 interactions has a unique mechanism of action: prevention of immune evasion of infected cells. The disclosure revolves around the inhibition of Nef-mediated functions, which is a distinct strategy since it targets a key function of Nef in its ability to directly modulate infected immune cell (macrophage) capacity to generate HIV-specific T cells from naïve T cell, potentially bringing forth a different class of drugs. The disclosure presents a new line of antiviral therapy through immune signaling restoration.

The host-virus interface is also less amenable to drug resistance than purely viral targets. The present disclosure discloses a method for preventing/treating HIV subtypes causing disease in a subject comprising: administering to a HIV infected subject a therapeutic dose of a compound selected the group consisting of Formula I. Formula II, and Formula III as described herein, wherein the therapeutic dose of the compound is in a range of 50 to 1000 μM. The present disclosure is intended to cover all the sub-ranges and individual values. The range can be 50 to 100 μM, or 100 to 1000 μM, or 150 to 900 μM, or 125 to 500 μM, or 500 to 900 μM, or 750 to 950 μM, or 75 to 150 μM, or 100 to 200 μM.

In an embodiment, the present disclosure provides a method for preventing/treating HIV subtypes causing disease in a subject comprising: administering to a HIV infected subject a therapeutic dose of a compound selected the group consisting of Formula I,

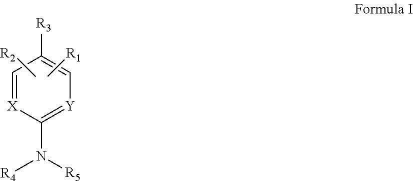

or its stereoisomers, pharmaceutically acceptable salts, polymorphs, solvates and hydrates thereof, wherein
R₁ and R₂ are independently selected from the group consisting of hydrogen, C₆₋₁₀ aryl, C₁₋₁₀ alkyl, C₂₋₁₀ alkenyl, C₂₋₁₀ alkynyl, and amino, wherein C₁₋₁₀ alkyl, C₂₋₁₀ alkenyl, C₂₋₁₀ alkynyl, and C₅₋₁₀ aryl are optionally substituted with one to four substituents independently selected from hydroxyl, halogen, C₁₋₁₀ alkyl, C₁₋₁₀ alkoxy, and C₂₋₁₀ heterocyclyl;
R₃ is C₆₋₁₀ aryl, wherein C₆₋₁₀ aryl is optionally substituted with one to four substituents independently selected from C₁₋₁₀ alkoxy, nitro, halogen or C₁₋₁₀ alkyl, wherein C₁₋₁₀ alkyl, and C₁₋₁₀ alkoxy is optionally substituted with one to four substituents selected from halogen, hydrogen, hydroxyl, C₁₋₁₀ alkyl, C₁₋₁₀ alkoxy or C₂₋₁₀ heterocyclyl;
R₄ and R₅ are independently selected from the group consisting of hydrogen, C₁₋₁₀ alkyl, and —N═CHC₆ aryl, wherein —N═CHC₆ aryl is further substituted with one to four halogen;
or R₄ and R₅ are joined to form a C₂₋₁₀ heterocyclyl or a C₂₋₁₀ heteroaryl, wherein C₂₋₁₀ heterocyclyl or C₂₋₁₀ heteroaryl is optionally substituted with one to four substituents independently selected from C₁₋₁₀ alkyl; and
X and Y are N;

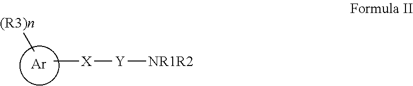

or its stereoisomers, pharmaceutically acceptable salts, polymorphs, solvates and hydrates thereof, wherein,
R₁, and R₂ are independently selected from the group consisting of hydrogen, C₁₋₁₀ alkyl, hydroxy, thio, amino, C₂₋₁₀ heterocyclyl, C₅₋₁₀ aryl, C₂₋₁₀ heteroaryl, acyl, amido, imido, sulfinyl, sulfonyl, carboxaldehyde, cyano, isocyano, azido, hydrazino, nitro, and halo; or
R₁ and R₂ are joined to form an optionally substituted monocyclic or a bicyclic ring, wherein C₁₋₁₀ alkyl, C₂₋₁₀ heterocyclyl, C₅₋₁₀ aryl, C₂₋₁₀ heteroaryl or monocyclic or a bicyclic ring are optionally substituted with one four substituents independently selected from hydroxy, C₁₋₁₀ alkyl, C₅₋₁₀ aryl, C₁₋₁₀ alkoxy, halogen, haloalkyl, perhaloalkyl, cyano, thio, and —CH₂-Ph-NH—COO-Ph-Me;
X is selected from O;
Y is C═O;
Ar is selected from C₁₋₁₀ alkyl, C₅₋₁₀ aryl or C₂₋₁₀ heteroaryl;
R₃ is absent or is selected from hydroxy, C₁₋₁₀ alkyl, C₅₋₁₀ aryl, C₂₋₁₀ heteroaryl, C₂₋₁₀ heterocyclyl, C₁₋₁₀ alkoxy, halogen, haloalkyl, perhaloalkyl, nitro, cyano, CH₂PhNHCOOPh-Me, NHCONHPh-(Cl)₂ or CONHNHSO₂Ph-Me; and
n is selected from 0-5; and

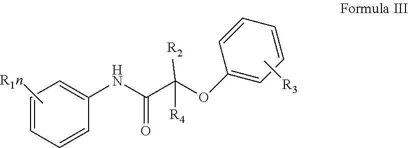

or its stereoisomers, pharmaceutically acceptable salts, polymorphs, solvates and hydrates thereof, wherein,
R₁ is independently selected from the group consisting of OH, COOH, and COORa, wherein Ra is C₁₋₁₀ alkyl;
R₂ and R₄ is independently selected from hydrogen or C₁₋₁₀ alkyl;
R₃ is selected from C₁₋₁₀ alkyl or halogen; and
n is selected from 1 to 5. In another embodiment of the present disclosure, the therapeutic dose of the compound is in a range of 50 to 1000 μM. In yet another embodiment of the present disclosure, the therapeutic dose of the compound is in a range of 50 to 100 μM. In an alternate embodiment of the present disclosure, the therapeutic dose of the compound is in a range of 100 to 1000 μM. In one another embodiment of the present disclosure, the therapeutic dose of the compound is in the range of 150 to 900 μM. In an alternate embodiment of the present disclosure, the therapeutic dose of the compound is in the range of 125 to 500 μM. In another embodiment of the present disclosure, the therapeutic dose of the compound is in the range of 500 to 900 μM. In another embodiment of the present disclosure, the therapeutic dose of the compound is in the range of 750 to 950 μM. In another embodiment of the present disclosure, the therapeutic dose of the compound is in the range of 75 to 150 μM. In another embodiment of the present disclosure, the therapeutic dose of the compound is in the range of 100 to 200 μM.

In an embodiment, the present disclosure provides a method for treating HIV subtypes causing disease in a subject comprising: administering to a HIV infected subject a therapeutic dose of a compound of Formula I,

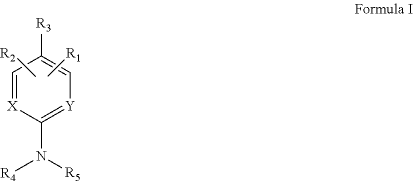

or its stereoisomers, pharmaceutically acceptable salts, polymorphs, solvates and hydrates thereof, wherein
R₁ and R₂ are independently selected from the group consisting of hydrogen, C₆₋₁₀ aryl, C₁₋₁₀ alkyl, and amino, wherein C₁₋₁₀ alkyl, and C₆₋₁₀ aryl are optionally substituted with one to four substituents independently selected from hydroxyl, halogen, C₁₋₁₀ alkyl, C₁₋₁₀ alkoxy, and C₂₋₁₀ heterocyclyl;
R₃ is C₆₋₁₀ aryl, wherein C₆₋₁₀ aryl is optionally substituted with one to four substituents independently selected from C₁₋₁₀ alkoxy, nitro, halogen or C₁₋₁₀ alkyl, wherein C₁₋₁₀ alkyl and C₁₋₁₀ alkoxy is optionally substituted with one to four substituents selected from halogen, hydrogen, hydroxyl, C₁₋₁₀ alkyl, C₁₋₁₀ alkoxy or C₂₋₁₀ heterocyclyl;
R₄ and R₅ are independently selected from the group consisting of hydrogen, C₁₋₁₀ alkyl, and —N═CHC₆ aryl, wherein —N═CHC₆ aryl is further substituted with one to four halogen;
or R₄ and R₅ are joined to form a C₂₋₁₀ heterocyclyl or a C₂₋₁₀ heteroaryl, wherein C₂₋₁₀ heterocyclyl or C₂₋₁₀ heteroaryl is optionally substituted with one to four substituents independently selected from C₁₋₁₀ alkyl; and X and Y are N.

In an embodiment, the present disclosure provides a method for treating HIV subtypes causing disease in a subject comprising: administering to a HIV infected subject a therapeutic dose of a compound of Formula I,

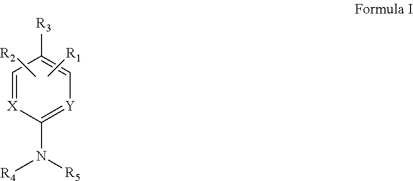

or its stereoisomers, pharmaceutically acceptable salts, polymorphs, solvates and hydrates thereof, wherein
R₁ and R₂ are independently selected from the group consisting of hydrogen, C₆₋₁₀ aryl, and C₁₋₁₀ alkyl, wherein C₁₋₁₀ alkyl, and C₆₋₁₀ aryl are optionally substituted with one to four substituents independently selected from hydroxyl, halogen, C₁₋₁₀ alkyl, and C₁₋₁₀ alkoxy;
R₃ is C₆₋₁₀ aryl, wherein C₆₋₁₀ aryl is optionally substituted with one to four substituents independently selected from C₁₋₁₀ alkoxy, nitro, halogen or C₁₋₁₀ alkyl, wherein C₁₋₁₀ alkyl and C₁₋₁₀ alkoxy is optionally substituted with one to four substituents selected from halogen, hydrogen, hydroxyl, C₁₋₁₀ alkyl, C₁₋₁₀ alkoxy or C₂₋₁₀ heterocyclyl;
R₄ and R₅ are independently selected from the group consisting of hydrogen, C₁₋₁₀ alkyl, and —N═CHC₆ aryl, wherein —N═CHC₆ aryl is further substituted with one to four halogen;
or R₄ and R₅ are joined to form a C₂₋₁₀ heterocyclyl or a C₂₋₁₀ heteroaryl, wherein C₂₋₁₀ heterocyclyl or C₂₋₁₀ heteroaryl is optionally substituted with one to four substituents independently selected from C₁₋₁₀ alkyl; and X and Y are N.

In an embodiment, the present disclosure provides a method for treating HIV subtypes causing disease in a subject comprising: administering to a HIV infected subject a therapeutic dose of a compound of Formula I,

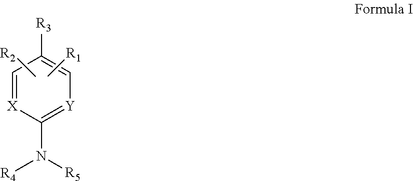

or its stereoisomers, pharmaceutically acceptable salts, polymorphs, solvates and hydrates thereof, wherein
R₁ and R₂ are independently selected from the group consisting of hydrogen, C₆ aryl, and C₁₋₅ alkyl, wherein C₁₋₅ alkyl, and C₆ aryl are optionally substituted with one to four substituents independently selected from hydroxyl, fluorine, C₁₋₅ alkyl or C₁₋₅ alkoxy;
R₃ is C₆ aryl, wherein C₆ aryl is optionally substituted with one to four substituents independently selected from C₁₋₅ alkoxy, nitro, halogen or C₁₋₅ alkyl, wherein C₁₋₅ alkyl and C₁₋₅ alkoxy is optionally substituted with one to four substituents selected from halogen, hydrogen, hydroxyl, C₁₋₅ alkyl, C₁₋₅ alkoxy or C₂₋₁₀heterocyclyl;
R₄ and R₅ are independently selected from the group consisting of hydrogen, C₁₋₅ alkyl, and —N═CHC₆ aryl, wherein —N═CHC₆ aryl is further substituted with one to four halogen;
or R₄ and R₅ are joined to form a C₂₋₁₀ heterocyclyl or a C₂₋₁₀ heteroaryl, wherein C₂₋₁₀ heterocyclyl or C₂₋₁₀ heteroaryl is optionally substituted with one to four substituents independently selected from C₁₋₅ alkyl; and X and Y are N.

In an embodiment, the present disclosure provides a method for treating HIV subtypes causing disease in a subject comprising: administering to a HIV infected subject a therapeutic dose of a compound of Formula I,

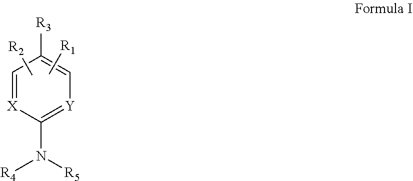

or its stereoisomers, pharmaceutically acceptable salts, polymorphs, solvates and hydrates thereof, wherein
R₁ and R₂ are independently selected from the group consisting of hydrogen, C₆ aryl, and CF₃, wherein C₆ aryl are optionally substituted with one to four substituents independently selected from hydroxyl, C₁₋₅ alkyl or C₁₋₅ alkoxy;
R₃ is C₆ aryl, wherein C₆ aryl is optionally substituted with one to four substituents independently selected from —OCH₃, —OCF₃, nitro, fluorine, CF₃;
R₄ and R₅ are independently selected from the group consisting of hydrogen, C₁₋₅ alkyl, and —N═CHC₆ aryl, wherein N═CHC₆ aryl is further substituted with one to four substituents selected from fluorine and bromine; or R₄ and R₅ are joined to form a C₂₋₁₀ heterocyclyl or a C₂₋₁₀ heteroaryl, wherein C₂₋₁₀ heterocyclyl or C₂₋₁₀ heteroaryl is optionally substituted with one to four substituents independently selected from C₁₋₅ alkyl; and X and Y are N.

In an embodiment, the present disclosure provides a method for treating HIV subtypes causing disease in a subject comprising: administering to a HIV infected subject a therapeutic dose of a compound of Formula I,

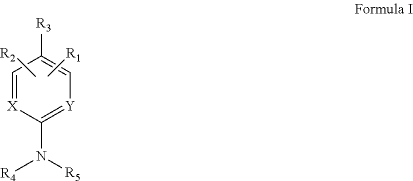

or its stereoisomers, pharmaceutically acceptable salts, polymorphs, solvates and hydrates thereof, wherein
R₁ and R₂ are independently selected from the group consisting of hydrogen, C₆₋₁₀ aryl, C₁₋₁₀ alkyl, C₂₋₁₀ alkenyl, C₂₋₁₀ alkynyl, and amino, wherein C₁₋₁₀ alkyl, C₂₋₁₀ alkenyl, C₂₋₁₀ alkynyl, and C₆₋₁₀ aryl are optionally substituted with one to four substituents independently selected from hydroxyl, halogen, C₁₋₁₀ alkyl or C₁₋₁₀ alkoxy, C₂₋₁₀ heterocyclyl;
R₃ is C₆₋₁₀ aryl, wherein C₆₋₁₀ aryl is optionally substituted with one to four substituents independently selected from C₁₋₁₀ alkoxy, nitro, C₁₋₁₀ alkyl, wherein C₁₋₁₀ alkyl and C₁₋₁₀ alkoxy is optionally substituted with one to four substituents selected from halogen, hydrogen, hydroxyl, C₁₋₁₀ alkyl, C₁₋₁₀ alkoxy or C₂₋₁₀ heterocyclyl;
R₄ and R₅ are independently selected from the group consisting of hydrogen, C₁₋₁₀ alkyl, and —N═CHC₆ aryl, wherein —N═CHC₆ aryl is further substituted with one to four halogen;
or R₄ and R₅ are joined to form a C₂₋₁₀ heterocyclyl or a C₂₋₁₀ heteroaryl, wherein C₂₋₁₀ heterocyclyl or C₂₋₁₀ heteroaryl is optionally substituted with one to four substituents independently selected from C₁₋₁₀ alkyl; and X and Y are N.

In an embodiment, the present disclosure provides a compound of Formula I or its stereoisomers, pharmaceutically acceptable salts, complexes, hydrates, solvates, tautomers, polymorphs, racemic mixtures, optically active forms and pharmaceutically active derivative thereof, which is selected from a group consisting of:

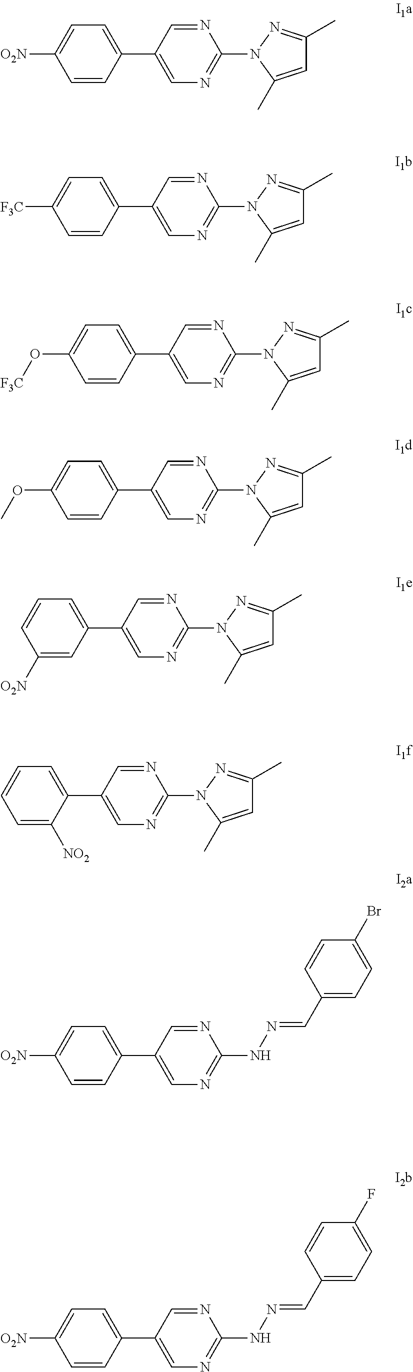

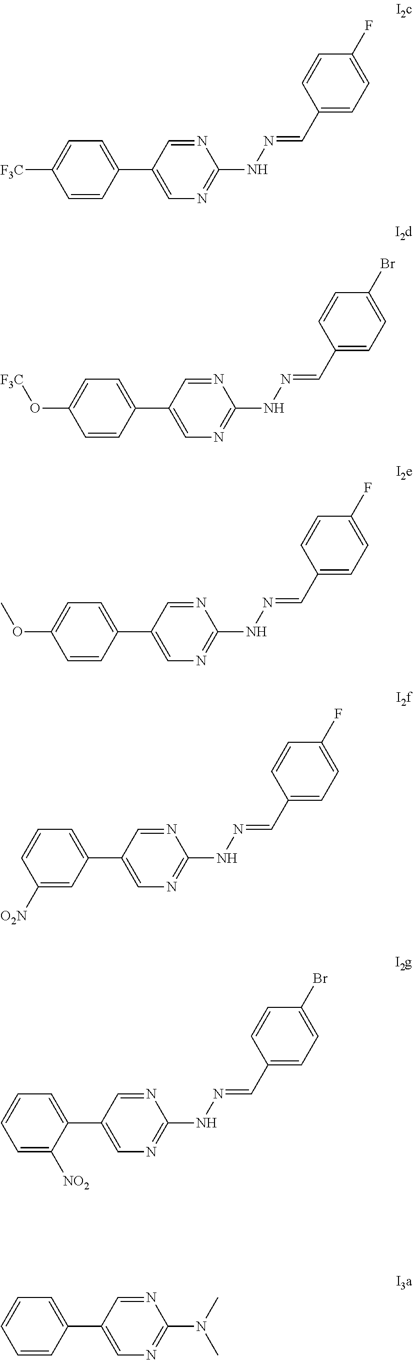 [SMILES: Fc1ccc(/C=N/Nc2ncc(-c3ccc(C(F)(F)F)cc3)cn2)cc1 ; FC(F)(F)Oc1ccc(-c2cnc(N/N=C/c3ccc(Br)cc3)nc2)cc1 ; COc1ccc(-c2cnc(N/N=C/c3ccc(F)cc3)nc2)cc1 ; O=[N+]([O-])c1cccc(-c2cnc(N/N=C/c3ccc(F)cc3)nc2)c1 ; O=[N+]([O-])c1ccccc1-c1cnc(N/N=C/c2ccc(Br)cc2)nc1 ; CN(C)c1ncc(-c2ccccc2)cn1]

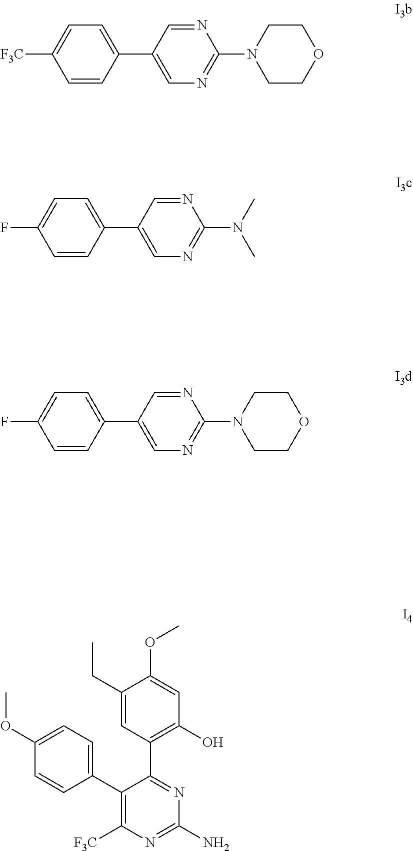 [SMILES: FC(F)(F)c1ccc(-c2cnc(N3CCOCC3)nc2)cc1 ; CN(C)c1ncc(-c2ccc(F)cc2)cn1 ; Fc1ccc(-c2cnc(N3CCOCC3)nc2)cc1 ; CCc1cc(-c2nc(N)nc(C(F)(F)F)c2-c2ccc(OC)cc2)c(O)cc1OC]

In an embodiment, the present disclosure provides a compound of Formula I or its stereoisomers, pharmaceutically acceptable salts, complexes, hydrates, solvates, tautomers, polymorphs, racemic mixtures, optically active forms and pharmaceutically active derivative thereof, which is selected from a group consisting of:

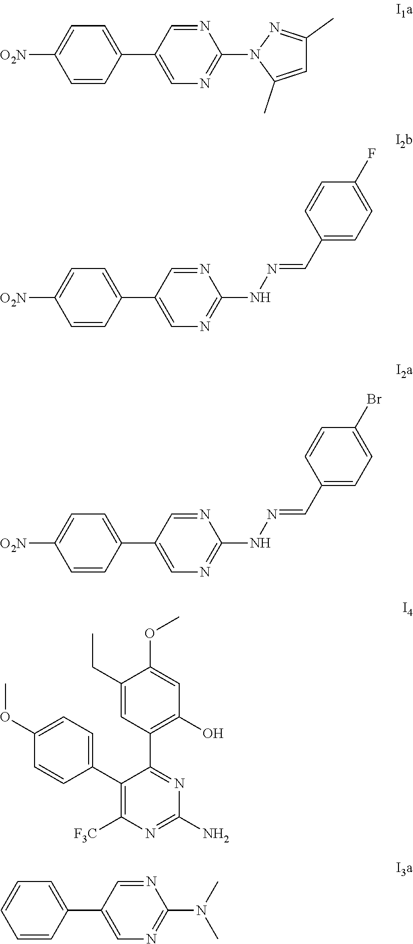

In an embodiment, the present disclosure provides a method for treating HIV subtypes causing disease in a subject comprising: administering to a HIV infected subject a therapeutic dose of a compound of Formula II

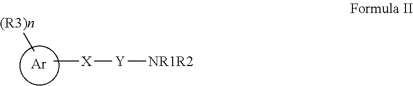

or its stereoisomers, pharmaceutically acceptable salts, polymorphs, solvates and hydrates thereof, wherein,
R₁, and R₂ are independently selected from the group consisting of hydrogen, C₁₋₁₀ alkyl, hydroxy, thio, amino, C₂₋₁₀ heterocyclyl, C₅₋₁₀ aryl, C₂₋₁₀ heteroaryl, acyl, amido, imido, sulfinyl, sulfonyl, carboxaldehyde, cyano, isocyano, azido, hydrazino, nitro, and halo; or
R₁ and R₂ are joined to form an optionally substituted monocyclic or a bicyclic ring, wherein C₁₋₁₀ alkyl, C₂₋₁₀ heterocyclyl, C₅₋₁₀ aryl, C₂₋₁₀ heteroaryl or monocyclic or a bicyclic ring are optionally substituted with one four substituents independently selected from hydroxy, C₁₋₁₀ alkyl, C₅₋₁₀ aryl, C₁₋₁₀ alkoxy, halogen, haloalkyl, perhaloalkyl, cyano, thio, and —CH₂-Ph-NH—COO-Ph-Me;
X is O;
Y is C═O;
Ar is selected from C₁₋₁₀ alkyl, C₅₋₁₀ aryl or C₂₋₁₀ heteroaryl;
R₃ is absent or is selected from hydroxy, C₁₋₁₀ alkyl, C₅₋₁₀ aryl, C₂₋₁₀ heteroaryl, C₂₋₁₀ heterocyclyl, C₁₋₁₀ alkoxy, halogen, haloalkyl, perhaloalkyl, nitro, cyano, CH₂PhNHCOOPh-Me, NHCONHPh-(Cl)₂ or CONHNHSO₂Ph-Me; and
n is selected from 0-5.

In an embodiment, the present disclosure provides a method for treating HIV subtypes causing disease in a subject comprising: administering to a HIV infected subject a therapeutic dose of a compound of Formula II

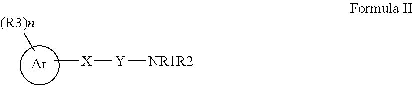

or its stereoisomers, pharmaceutically acceptable salts, polymorphs, solvates and hydrates thereof, wherein,
R₁, and R₂ are independently selected from the group consisting of hydrogen, C₁₋₁₀ alkyl, hydroxy, thio, amino, C₂₋₁₀ heterocyclyl, C₅₋₁₀ aryl, C₂₋₁₀ heteroaryl, acyl, amido, imido, sulfinyl, sulfonyl, carboxaldehyde, cyano, isocyano, azido, hydrazino, nitro, and halo; or
R₁ and R₂ are joined to form an optionally substituted monocyclic or a bicyclic ring, wherein C₁₋₁₀ alkyl, C₂₋₁₀ heterocyclyl, C₅₋₁₀ aryl, C₂₋₁₀ heteroaryl or monocyclic or a bicyclic ring are optionally substituted with one four substituents independently selected from hydroxy, C₁₋₁₀ alkyl, C₅₋₁₀ aryl, C₁₋₁₀ alkoxy, halogen, haloalkyl, perhaloalkyl, cyano, thio, and —CH₂-Ph-NH—COO-Ph-Me;
X is O;
Y is C═O;
Ar is selected from C₁₋₁₀ alkyl, C₅₋₁₀ aryl or C₂₋₁₀ heteroaryl;
R₃ is absent or is selected from hydroxy, C₁₋₁₀ alkyl, C₅₋₁₀ aryl, C₂₋₁₀ heteroaryl, C₂₋₁₀ heterocyclyl, C₁₋₁₀ alkoxy, halogen, haloalkyl, perhaloalkyl, nitro, cyano, CH₂PhNHCOOPh-Me, NHCONHPh-(Cl)₂ or CONHNHSO₂Ph-Me; and
n is selected from 0-5.

In an embodiment, the present disclosure provides a method for treating HIV subtypes causing disease in a subject comprising: administering to a HIV infected subject a therapeutic dose of a compound of Formula II

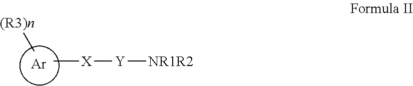

or its stereoisomers, pharmaceutically acceptable salts, polymorphs, solvates and hydrates thereof, wherein,
R₁, and R₂ are independently selected from the group consisting of hydrogen, C₁₋₁₀ alkyl, hydroxy, thio, amino, C₂₋₁₀ heterocyclyl, C₅₋₁₀ aryl, C₂₋₁₀ heteroaryl, acyl, amido, imido, sulfinyl, sulfonyl, carboxaldehyde, cyano, isocyano, azido, hydrazino, nitro, and halo; or
R₁ and R₂ are joined to form an optionally substituted monocyclic or a bicyclic ring, wherein C₁₋₁₀ alkyl, C₂₋₁₀ heterocyclyl, C₅₋₁₀ aryl, C₂₋₁₀ heteroaryl or monocyclic or a bicyclic ring are optionally substituted with one four substituents independently selected from hydroxy, C₁₋₁₀ alkyl, C₅₋₁₀ aryl, C₁₋₁₀ alkoxy, halogen, haloalkyl, perhaloalkyl, cyano, thio, and —CH₂-Ph-NH—COO-Ph-Me;
X is O;
Y is C═O;
Ar is selected from C₅₋₁₀aryl or C₂₋₁₀heteroaryl;
R₃ is absent or is selected from hydroxy, C₁₋₁₀ alkyl, C₅₋₁₀ aryl, C₂₋₁₀ heteroaryl, C₂₋₁₀ heterocyclyl, C₁₋₁₀ alkoxy, halogen, haloalkyl, perhaloalkyl, nitro, cyano, CH₂PhNHCOOPh-Me, NHCONHPh-(Cl)₂ or CONHNHSO₂Ph-Me; and
n is selected from 0-5.

In an embodiment, the present disclosure provides a method for treating HIV subtypes causing disease in a subject comprising: administering to a HIV infected subject a therapeutic dose of a compound of Formula II

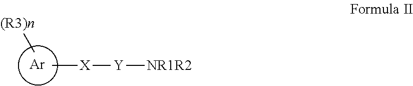

or its stereoisomers, pharmaceutically acceptable salts, polymorphs, solvates and hydrates thereof, wherein,
R₁, and R₂ are independently selected from the group consisting of hydrogen, C₁₋₁₀ alkyl, hydroxy, thio, amino, C₂₋₁₀ heterocyclyl, C₅₋₁₀ aryl, C₂₋₁₀ heteroaryl, acyl, amido, imido, sulfinyl, sulfonyl, carboxaldehyde, cyano, isocyano, azido, hydrazino, nitro, and halo; or
R₁ and R₂ are joined to form an optionally substituted monocyclic or a bicyclic ring, wherein C₁₋₁₀ alkyl, C₂₋₁₀ heterocyclyl, C₅₋₁₀ aryl, C₂₋₁₀ heteroaryl or monocyclic or a bicyclic ring are optionally substituted with one four substituents independently selected from hydroxy, C₁₋₁₀ alkyl, C₅₋₁₀ aryl, C₁₋₁₀ alkoxy, halogen, haloalkyl, perhaloalkyl, cyano, thio, and —CH₂-Ph-NH—COO-Ph-Me;
X is O;
Y is C═O;
Ar is C₅₋₁₀ aryl;
R₃ is absent or is selected from hydroxy, C₁₋₁₀ alkyl, C₅₋₁₀ aryl, C₂₋₁₀ heteroaryl, C₂₋₁₀ heterocyclyl, C₁₋₁₀ alkoxy, halogen, haloalkyl, perhaloalkyl, nitro, cyano, CH₂PhNHCOOPh-Me, NHCONHPh-(Cl)₂ or CONHNHSO₂Ph-Me; and
n is selected from 0-5.

In an embodiment, the present disclosure provides a compound of Formula II or its stereoisomers, pharmaceutically acceptable salts, complexes, hydrates, solvates, tautomers, polymorphs, racemic mixtures, optically active forms and pharmaceutically active derivative thereof, which is selected from a group consisting of:

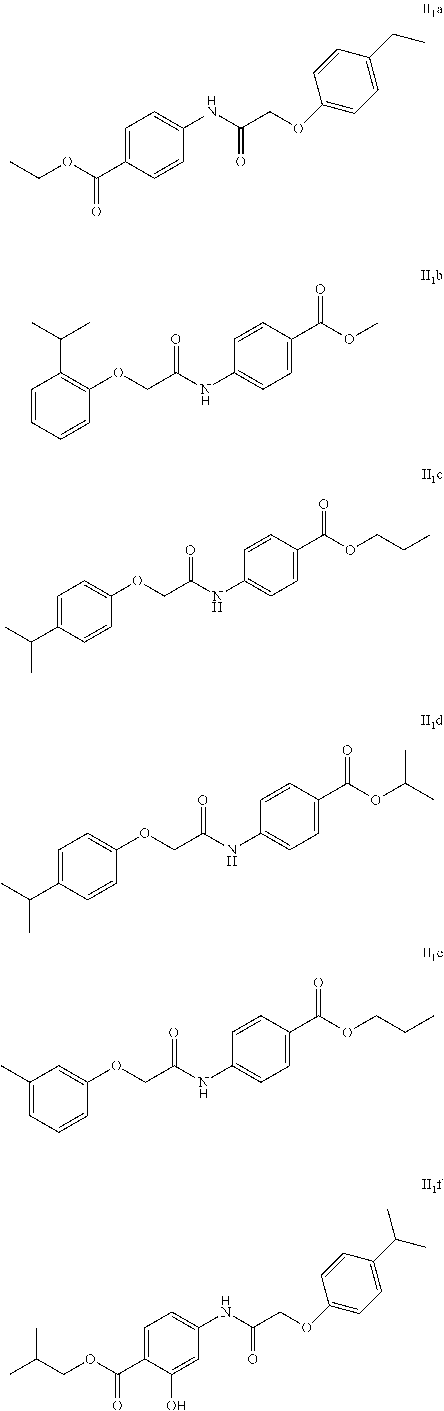

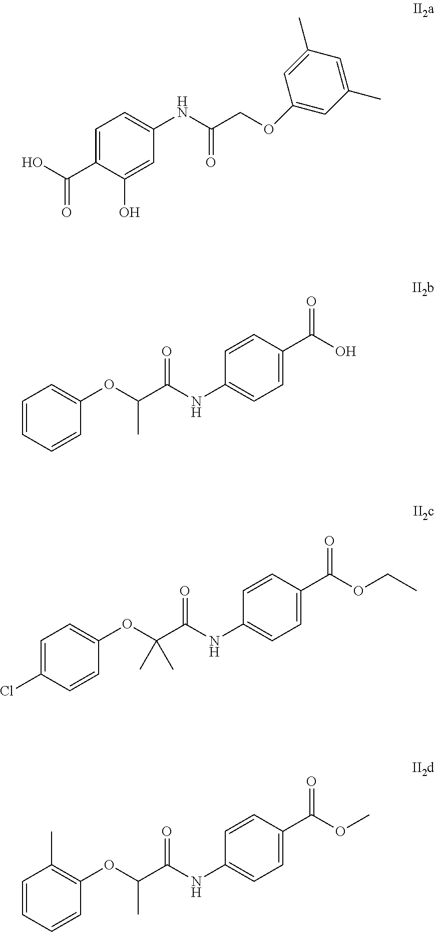 [SMILES: Cc1cc(C)cc(OCC(=O)Nc2ccc(C(=O)O)c(O)c2)c1 ; CC(Oc1ccccc1)C(=O)Nc1ccc(C(=O)O)cc1 ; CCOC(=O)c1ccc(NC(=O)C(C)(C)Oc2ccc(Cl)cc2)cc1 ; COC(=O)c1ccc(NC(=O)C(C)Oc2ccccc2C)cc1]

In an embodiment, the present disclosure provides a method for treating HIV subtypes causing disease in a subject comprising: administering to a HIV infected subject a therapeutic dose of a compound of Formula III

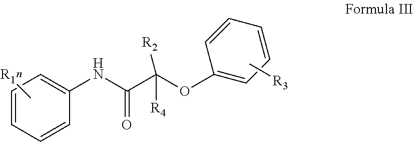

or its stereoisomers, pharmaceutically acceptable salts, polymorphs, solvates and hydrates thereof, wherein,
R₁ is independently selected from the group consisting of OH, COOH, and COORa, wherein Ra is C₁₋₁₀ alkyl;
R₂ and R₄ is independently selected from hydrogen or C₁₋₁₀ alkyl;
R₃ is selected from C₁₋₁₀ alkyl or halogen; and
n is selected from 1 to 5.

In an embodiment, the present disclosure provides a method for treating HIV subtypes causing disease in a subject comprising: administering to a HIV infected subject a therapeutic dose of a compound of Formula III

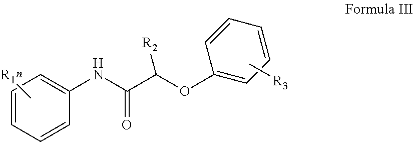

or its stereoisomers, pharmaceutically acceptable salts, polymorphs, solvates and hydrates thereof, wherein,
R₁ is independently selected from the group consisting of OH, COOH, and COORa, wherein Ra is C₁₋₅alkyl;
R₂ and R₄ is independently selected from hydrogen or C₁₋₅ alkyl;
R₃ is selected from C₁₋₅alkyl or chlorine; and n is selected from 1 to 2.

In an embodiment, the present disclosure provides a compound of Formula III or its stereoisomers, pharmaceutically acceptable salts, complexes, hydrates, solvates, tautomers, polymorphs, racemic mixtures, optically active forms and pharmaceutically active derivative thereof, which is selected from a group consisting of:

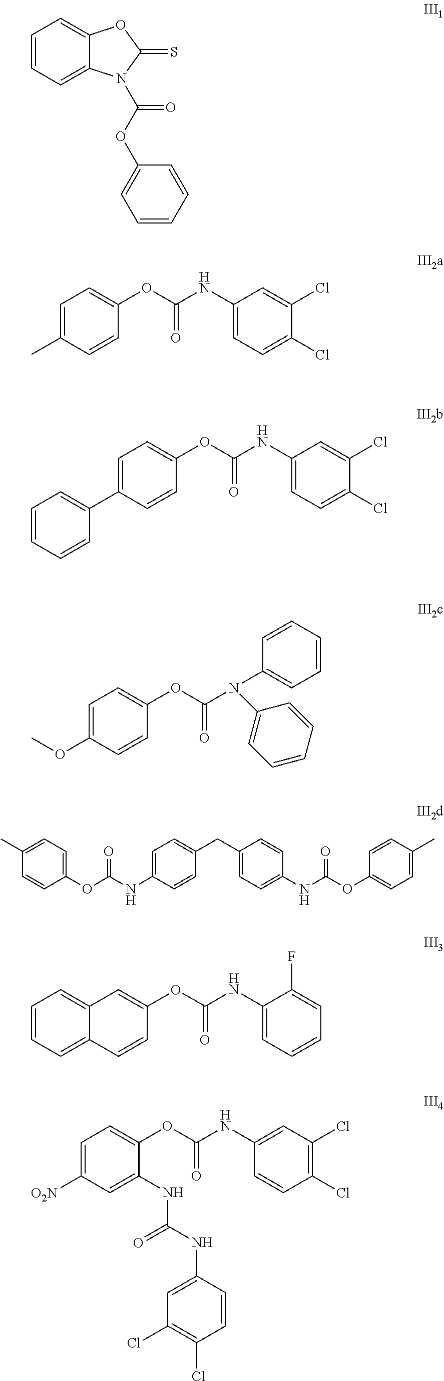

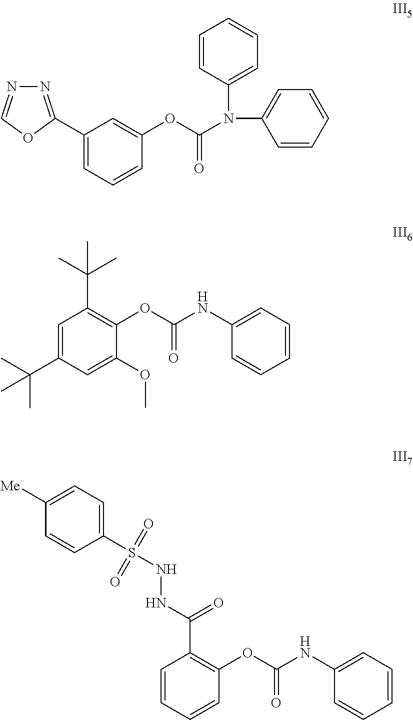 [SMILES: O=C(Oc1cccc(-c2nnco2)c1)N(c1ccccc1)c1ccccc1 ; COc1cc(C(C)(C)C)cc(C(C)(C)C)c1OC(=O)Nc1ccccc1 ; Cc1ccc(S(=O)(=O)NNC(=O)c2ccccc2OC(=O)Nc2ccccc2)cc1]

In an embodiment, the present disclosure provides a method for preventing HIV subtypes causing disease in a subject in need thereof comprising: administering to a HIV high risk subject a relevant dose of a compound selected from the group consisting of Formula I,

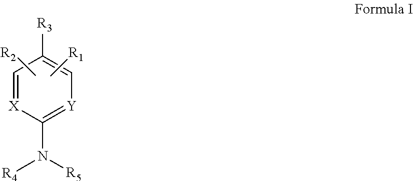

or its stereoisomers, pharmaceutically acceptable salts, polymorphs, solvates and hydrates thereof, wherein
R₁ and R₂ are independently selected from the group consisting of hydrogen, C₆₋₁₀ aryl, C₁₋₁₀ alkyl, C₂₋₁₀ alkenyl, C₂₋₁₀ alkynyl, and amino, wherein C₁₋₁₀ alkyl, C₂₋₁₀ alkenyl, C₂₋₁₀ alkynyl, and C₆₋₁₀ aryl are optionally substituted with one to four substituents independently selected from hydroxyl, halogen, C₁₋₁₀ alkyl, C₁₋₁₀ alkoxy or C₂₋₁₀ heterocyclyl;
R₃ is C₆₋₁₀ aryl, wherein C₆₋₁₀ aryl is optionally substituted with one to four substituents independently selected from C₁₋₁₀ alkoxy, nitro, halogen or C₁₋₁₀ alkyl, wherein C₁₋₁₀ alkyl and C₁₋₁₀ alkoxy is optionally substituted with one to four substituents selected from halogen, hydrogen, hydroxyl, C₁₋₁₀ alkyl, C₁₋₁₀ alkoxy, C₂₋₁₀ heterocyclyl;
R₄ and R₅ are independently selected from the group consisting of hydrogen, C₁₋₁₀ alkyl, and —N═CHC₆ aryl, wherein —N═CHC₆ aryl is further substituted with one to four halogen;
or R₄ and R₅ are joined to form a C₂₋₁₀heterocyclyl or a C₂₋₁₀ heteroaryl, wherein C₂₋₁₀ heterocyclyl or C₂₋₁₀ heteroaryl is optionally substituted with one to four substituents independently selected from C₁₋₁₀ alkyl; and X and Y are N;

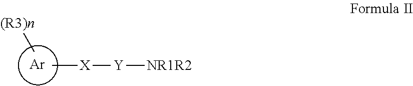

or its stereoisomers, pharmaceutically acceptable salts, polymorphs, solvates and hydrates thereof, wherein,
R₁, and R₂ are independently selected from the group consisting of hydrogen, C₁₋₁₀ alkyl, hydroxy, thio, amino, C₂₋₁₀ heterocyclyl, C₅₋₁₀ aryl, C₂₋₁₀ heteroaryl, acyl, amido, imido, sulfinyl, sulfonyl, carboxaldehyde, cyano, isocyano, azido, hydrazino, nitro, and halo; or
R₁ and R₂ are joined to form an optionally substituted monocyclic or a bicyclic ring, wherein C₁₋₁₀ alkyl, C₂₋₁₀ heterocyclyl, C₅₋₁₀ aryl, C₂₋₁₀ heteroaryl or monocyclic or a bicyclic ring are optionally substituted with one four substituents independently selected from hydroxy, C₁₋₁₀ alkyl, C₅₋₁₀ aryl, C₁₋₁₀ alkoxy, halogen, haloalkyl, perhaloalkyl, cyano, thio, and —CH₂-Ph-NH—COO-Ph-Me;
X is O;
Y is C═O;
Ar is selected from C₁₋₁₀ alkyl, C₅₋₁₀ aryl or C₂₋₁₀ heteroaryl;
R₃ is absent or is selected from hydroxy, C₁₋₁₀ alkyl, C₅₋₁₀ aryl, C₂₋₁₀ heteroaryl, C₂₋₁₀ heterocyclyl, C₁₋₁₀ alkoxy, halogen, haloalkyl, perhaloalkyl, nitro, cyano, CH₂PhNHCOOPh-Me, NHCONHPh-(Cl)₂ or CONHNHSO₂Ph-Me; and n is selected from 0-5; and

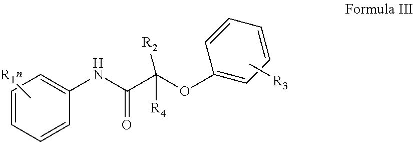

or its stereoisomers, pharmaceutically acceptable salts, polymorphs, solvates and hydrates thereof, wherein,
R₁ is independently selected from the group consisting of OH, COOH, and COORa, wherein Ra is C₁₋₁₀ alkyl;
R₂ and R₄ is independently selected from hydrogen or C₁₋₁₀ alkyl;
R₃ is selected from C₁₋₁₀ alkyl or halogen; and
n is selected from 1 to 5. In another embodiment of the present disclosure, the therapeutic dose of the compound is in the range of 50 to 1000 μM. In yet another embodiment of the present disclosure, the therapeutic dose of the compound is in the range of 50 to 100 μM. In an another embodiment of the present disclosure, the therapeutic dose of the compound is in the range of 100 to 1000 μM. In an alternate embodiment of the present disclosure, the therapeutic dose of the compound is in the range of 150 to 900 μM. In one another embodiment of the present disclosure, the therapeutic dose of the compound is in the range of 125 to 500 μM. In an embodiment, the present disclosure provides a method for preventing HIV subtypes causing disease in a subject in need thereof comprising: administering to a HIV high risk subject a relevant dose of a compound selected from the group consisting of Formula I,

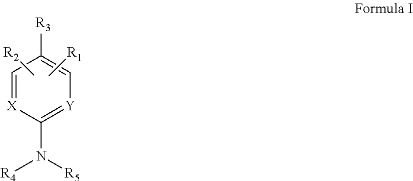

or its stereoisomers, pharmaceutically acceptable salts, polymorphs, solvates and hydrates thereof, wherein
R₁ and R₂ are independently selected from the group consisting of hydrogen, C₆₋₁₀ aryl, C₁₋₁₀ alkyl, and amino, wherein C₁₋₁₀ alkyl, and C₆₋₁₀ aryl are optionally substituted with one to four substituents independently selected from hydroxyl, halogen, C₁₋₁₀ alkyl, C₁₋₁₀ alkoxy or C₂₋₁₀heterocyclyl;
R₃ is C₆₋₁₀ aryl, wherein C₆₋₁₀ aryl is optionally substituted with one to four substituents independently selected from C₁₋₁₀ alkoxy, nitro, halogen or C₁₋₁₀ alkyl, wherein C₁₋₁₀ alkyl and C₁₋₁₀ alkoxy is optionally substituted with one to four substituents selected from halogen, hydrogen, hydroxyl, C₁₋₁₀ alkyl, C₁₋₁₀ alkoxy or C₂₋₁₀heterocyclyl;
R₄ and R₅ are independently selected from the group consisting of hydrogen, C₁₋₁₀ alkyl, and —N═CHC₆ aryl, wherein —N═CHC₆ aryl is further substituted with one to four halogen;
or R₄ and R₅ are joined to form a C₂₋₁₀ heterocyclyl or a C₂₋₁₀ heteroaryl, wherein C₂₋₁₀ heterocyclyl or C₂₋₁₀ heteroaryl is optionally substituted with one to four substituents independently selected from C₁₋₁₀ alkyl; and X and Y are N.

In an embodiment, the present disclosure provides a method for preventing HIV subtypes causing disease in a subject in need thereof comprising: administering to a HIV high risk subject a relevant dose of a compound selected from the group consisting of Formula I,

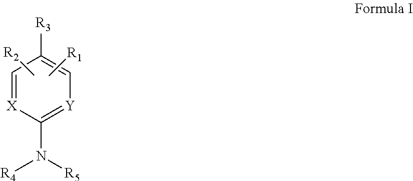

or its stereoisomers, pharmaceutically acceptable salts, polymorphs, solvates and hydrates thereof, wherein
R₁ and R₂ are independently selected from the group consisting of hydrogen, C₆₋₁₀ aryl, and C₁₋₁₀ alkyl, wherein C₁₋₁₀ alkyl, and C₆₋₁₀ aryl are optionally substituted with one to four substituents independently selected from hydroxyl, halogen, C₁₋₁₀ alkyl or C₁₋₁₀ alkoxy;
R₃ is C₆₋₁₀ aryl, wherein C₆₋₁₀ aryl is optionally substituted with one to four substituents independently selected from C₁₋₁₀ alkoxy, nitro, halogen, C₁₋₁₀ alkyl, wherein C₁₋₁₀ alkyl and C₁₋₁₀ alkoxy is optionally substituted with one to four substituents selected from halogen, hydrogen, hydroxyl, C₁₋₁₀ alkyl, C₁₋₁₀ alkoxy or C₂₋₁₀ heterocyclyl;
R₄ and R₅ are independently selected from the group consisting of hydrogen, C₁₋₁₀ alkyl, and —N═CHC₆ aryl, wherein —N═CHC₆ aryl is further substituted with one to four halogen, or R₄ and R₅ are joined to form a C₂₋₁₀ heterocyclyl or a C₂₋₁₀ heteroaryl, wherein C₂₋₁₀ heterocyclyl or C₂₋₁₀ heteroaryl is optionally substituted with one to four substituents independently selected from C₁₋₁₀ alkyl; and X and Y are N.

In an embodiment, the present disclosure provides a method for preventing HIV subtypes causing disease in a subject in need thereof comprising: administering to a HIV high risk subject a relevant dose of a compound selected from the group consisting of Formula I,

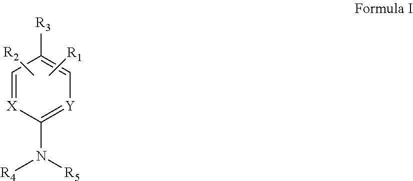

or its stereoisomers, pharmaceutically acceptable salts, polymorphs, solvates and hydrates thereof, wherein
R₁ and R₂ are independently selected from the group consisting of hydrogen, C₆ aryl, and C₁₋₅ alkyl, wherein C₁₋₅ alkyl, and C₆ aryl are optionally substituted with one to four substituents independently selected from hydroxyl, fluorine, Ci-5 alkyl or C₁₋₅ alkoxy;
R₃ is C₆ aryl, wherein C₆ aryl is optionally substituted with one to four substituents independently selected from C₁₋₅alkoxy, nitro, halogen, C₁₋₅ alkyl, wherein C₁₋₅ alkyl and C₁₋₅ alkoxy is optionally substituted with one to four substituents selected from halogen, hydrogen, hydroxyl, C₁₋₅ alkyl, C₁₋₅ alkoxy or C₂₋₁₀ heterocyclyl;
R₄ and R₅ are independently selected from the group consisting of hydrogen, C₁₋₅ alkyl, and —N═CHC₆ aryl, wherein —N═CHC₆ aryl is further substituted with one to four halogen;
or R₄ and R₅ are joined to form a C₂₋₁₀ heterocyclyl or a C₂₋₁₀ heteroaryl, wherein C₂₋₁₀ heterocyclyl or C₂₋₁₀ heteroaryl is optionally substituted with one to four substituents independently selected from C₁₋₅ alkyl; and X and Y are N.

In an embodiment, the present disclosure provides a method for preventing HIV subtypes causing disease in a subject in need thereof comprising: administering to a HIV high risk subject a relevant dose of a compound selected from the group consisting of Formula I,

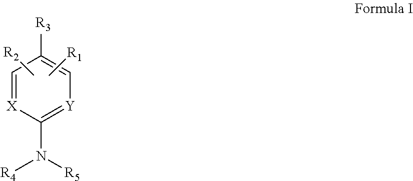

or its stereoisomers, pharmaceutically acceptable salts, polymorphs, solvates and hydrates thereof, wherein
R₁ and R₂ are independently selected from the group consisting of hydrogen, C₆ aryl, and CF₃, wherein C₆ aryl is optionally substituted with one to four substituents independently selected from hydroxyl, C₁₋₅ alkyl or C₁₋₅ alkoxy;
R₃ is C₆ aryl, wherein C₆ aryl is optionally substituted with one to four substituents independently selected from —OCH₃, —OCF₃, nitro, fluorine or CF₃;
R₄ and R₅ are independently selected from the group consisting of hydrogen, C₁₋₅ alkyl, and —N═CHC₆ aryl, wherein —N═CHC₆ aryl is further substituted with one to four substituents selected from fluorine and bromine; or R₄ and R₅ are joined to form a C₂₋₁₀ heterocyclyl or a C₂₋₁₀ heteroaryl, wherein C₂₋₁₀ heterocyclyl or C₂₋₁₀ heteroaryl is optionally substituted with one to four substituents independently selected from C₁₋₅ alkyl; and X and Y are N.

In an embodiment, the present disclosure provides a method for preventing HIV subtypes causing disease in a subject in need thereof comprising: administering to a HIV high risk subject a relevant dose of a compound selected from the group consisting of Formula I,

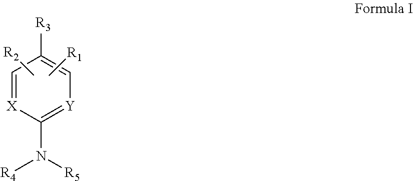

or its stereoisomers, pharmaceutically acceptable salts, polymorphs, solvates and hydrates thereof, wherein
R₁ and R₂ are independently selected from the group consisting of hydrogen, C₆₋₁₀ aryl, C₁₋₁₀ alkyl, C₂₋₁₀ alkenyl, C₂₋₁₀ alkynyl, and amino, wherein C₁₋₁₀ alkyl, C₂₋₁₀ alkenyl, C₂₋₁₀ alkynyl, and C₆₋₁₀ aryl are optionally substituted with one to four substituents independently selected from hydroxyl, halogen, C₁₋₁₀ alkyl, C₁₋₁₀ alkoxy or C₂₋₁₀ heterocyclyl;
R₃ is C₆₋₁₀ aryl, wherein C₆₋₁₀ aryl is optionally substituted with one to four substituents independently selected from C₁₋₁₀ alkoxy, nitro or C₁₋₁₀ alkyl, wherein C₁₋₁₀ alkyl and C₁₋₁₀ alkoxy is optionally substituted with one to four substituents selected from halogen, hydrogen, hydroxyl, C₁₋₁₀ alkyl, C₁₋₁₀ alkoxy or C₂₋₁₀heterocyclyl;
R₄ and R₅ are independently selected from the group consisting of hydrogen, C₁₋₁₀ alkyl, and —N═CHC₆ aryl, wherein —N═CHC₆ aryl is further substituted with one to four halogen, or R₄ and R₅ are joined to form a C₂₋₁₀ heterocyclyl or a C₂₋₁₀ heteroaryl, wherein C₂₋₁₀ heterocyclyl or C₂₋₁₀ heteroaryl is optionally substituted with one to four substituents independently selected from C₁₋₁₀ alkyl; and X and Y are N.

In an embodiment, the present disclosure provides a method for preventing HIV subtypes causing disease in a subject in need thereof comprising: administering to a HIV high risk subject a relevant dose of a compound selected from the group consisting of Formula II

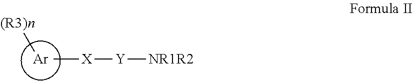

or its stereoisomers, pharmaceutically acceptable salts, polymorphs, solvates and hydrates thereof, wherein,
R₁, and R₂ are independently selected from the group consisting of hydrogen, C₁₋₁₀ alkyl, hydroxy, thio, amino, C₂₋₁₀ heterocyclyl, C₅₋₁₀ aryl, C₂₋₁₀ heteroaryl, acyl, amido, imido, sulfinyl, sulfonyl, carboxaldehyde, cyano, isocyano, azido, hydrazino, nitro, and halo; or
R₁ and R₂ are joined to form an optionally substituted monocyclic or a bicyclic ring, wherein C₁₋₁₀ alkyl, C₂₋₁₀ heterocyclyl, C₅₋₁₀ aryl, C₂₋₁₀ heteroaryl or monocyclic or a bicyclic ring are optionally substituted with one four substituents independently selected from hydroxy, C₁₋₁₀ alkyl, C₅₋₁₀ aryl, C₁₋₁₀ alkoxy, halogen, haloalkyl, perhaloalkyl, cyano, thio, and —CH₂-Ph-NH—COO-Ph-Me;
X is O;
Y is C═O;
Ar is selected from C₁₋₁₀ alkyl, C₅₋₁₀ aryl or C₂₋₁₀ heteroaryl;
R₃ is absent or is selected from hydroxy, C₁₋₁₀ alkyl, C₅₋₁₀ aryl, C₂₋₁₀ heteroaryl, C₂₋₁₀ heterocyclyl, C₁₋₁₀ alkoxy, halogen, haloalkyl, perhaloalkyl, nitro, cyano, CH₂PhNHCOOPh-Me, NHCONHPh-(Cl)₂ or CONHNHSO₂Ph-Me; and
n is selected from 0-5.

In an embodiment, the present disclosure provides a method for preventing HIV subtypes causing disease in a subject in need thereof comprising: administering to a HIV high risk subject a relevant dose of a compound selected from the group consisting of Formula II

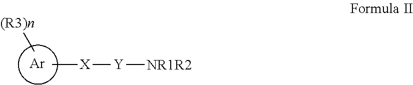

or its stereoisomers, pharmaceutically acceptable salts, polymorphs, solvates and hydrates thereof, wherein,
R₁, and R₂ are independently selected from the group consisting of hydrogen, C₁₋₁₀ alkyl, hydroxy, thio, amino, C₂₋₁₀ heterocyclyl, C₅₋₁₀ aryl, C₂₋₁₀ heteroaryl, acyl, amido, imido, sulfinyl, sulfonyl, carboxaldehyde, cyano, isocyano, azido, hydrazino, nitro, and halo; or
R₁ and R₂ are joined to form an optionally substituted monocyclic or a bicyclic ring, wherein C₁₋₁₀ alkyl, C₂₋₁₀ heterocyclyl, C₅₋₁₀ aryl, C₂₋₁₀ heteroaryl or monocyclic or a bicyclic ring are optionally substituted with one four substituents independently selected from hydroxy, C₁₋₁₀ alkyl, C₅₋₁₀ aryl, C₁₋₁₀ alkoxy, halogen, haloalkyl, perhaloalkyl, cyano, thio, and —CH₂-Ph-NH—COO-Ph-Me;
X is O;
Y is C═O;
Ar is selected from C₁₋₁₀ alkyl, C₅₋₁₀ aryl or C₂₋₁₀ heteroaryl;
R₃ is absent or is selected from hydroxy, C₁₋₁₀ alkyl, C₅₋₁₀ aryl, C₂₋₁₀ heteroaryl, C₂₋₁₀ heterocyclyl, C₁₋₁₀ alkoxy, halogen, haloalkyl, perhaloalkyl, nitro, cyano, CH₂PhNHCOOPh-Me, NHCONHPh-(Cl)₂ or CONHNHSO₂Ph-Me; and
n is selected from 0-5.

In an embodiment, the present disclosure provides a method for preventing HIV subtypes causing disease in a subject in need thereof comprising: administering to a HIV high risk subject a relevant dose of a compound selected from the group consisting of Formula II

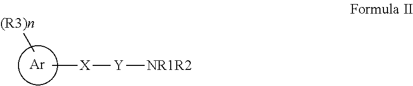

or its stereoisomers, pharmaceutically acceptable salts, polymorphs, solvates and hydrates thereof, wherein,
R₁, and R₂ are independently selected from the group consisting of hydrogen, C₁₋₁₀ alkyl, hydroxy, thio, amino, C₂₋₁₀ heterocyclyl, C₅₋₁₀ aryl, C₂₋₁₀ heteroaryl, acyl, amido, imido, sulfinyl, sulfonyl, carboxaldehyde, cyano, isocyano, azido, hydrazino, nitro, and halo; or
R₁ and R₂ are joined to form an optionally substituted monocyclic or a bicyclic ring, wherein C₁₋₁₀ alkyl, C₂₋₁₀ heterocyclyl, C₅₋₁₀ aryl, C₂₋₁₀ heteroaryl or monocyclic or a bicyclic ring are optionally substituted with one four substituents independently selected from hydroxy, C₁₋₁₀ alkyl, C₅₋₁₀ aryl, C₁₋₁₀ alkoxy, halogen, haloalkyl, perhaloalkyl, cyano, thio, and —CH₂-Ph-NH—COO-Ph-Me;
X is O;
Y is C═O;
Ar is selected from C₅₋₁₀ aryl or C₂₋₁₀ heteroaryl;
R₃ is absent or is selected from hydroxy, C₁₋₁₀ alkyl, C₅₋₁₀ aryl, C₂₋₁₀ heteroaryl, C₂₋₁₀ heterocyclyl, C₁₋₁₀ alkoxy, halogen, haloalkyl, perhaloalkyl, nitro, cyano, CH₂PhNHCOOPh-Me, NHCONHPh-(Cl)₂ or CONHNHSO₂Ph-Me; and n is selected from 0-5.

In an embodiment, the present disclosure provides a method for preventing HIV subtypes causing disease in a subject in need thereof comprising: administering to a HIV high risk subject a relevant dose of a compound selected from the group consisting of Formula II

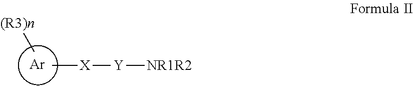

or its stereoisomers, pharmaceutically acceptable salts, polymorphs, solvates and hydrates thereof, wherein,
R₁, and R₂ are independently selected from the group consisting of hydrogen, C₁₋₁₀ alkyl, hydroxy, thio, amino, C₂₋₁₀ heterocyclyl, C₅₋₁₀ aryl, C₂₋₁₀ heteroaryl, acyl, amido, imido, sulfinyl, sulfonyl, carboxaldehyde, cyano, isocyano, azido, hydrazino, nitro, and halo; or
R₁ and R₂ are joined to form an optionally substituted monocyclic or a bicyclic ring, wherein C₁₋₁₀ alkyl, C₂₋₁₀ heterocyclyl, C₅₋₁₀ aryl, C₂₋₁₀ heteroaryl or monocyclic or a bicyclic ring are optionally substituted with one four substituents independently selected from hydroxy, C₁₋₁₀ alkyl, C₅₋₁₀ aryl, C₁₋₁₀ alkoxy, halogen, haloalkyl, perhaloalkyl, cyano, thio, and —CH₂-Ph-NH—COO-Ph-Me;
X is O;
Y is C═O;
Ar is C₅₋₁₀ aryl;
R₃ is absent or is selected from hydroxy, C₁₋₁₀ alkyl, C₅₋₁₀ aryl, C₂₋₁₀ heteroaryl, C₂₋₁₀ heterocyclyl, C₁₋₁₀ alkoxy, halogen, haloalkyl, perhaloalkyl, nitro, cyano, CH₂PhNHCOOPh-Me, NHCONHPh-(Cl)₂ or CONHNHSO₂Ph-Me; and
n is selected from 0-5.

In an embodiment, the present disclosure provides a method for preventing HIV subtypes causing disease in a subject in need thereof comprising: administering to a HIV high risk subject a relevant dose of a compound selected from the group consisting of Formula III

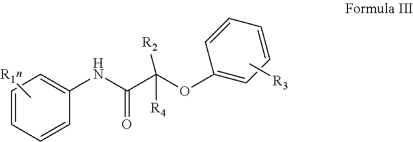

or its stereoisomers, pharmaceutically acceptable salts, polymorphs, solvates and hydrates thereof, wherein,
R₁ is independently selected from the group consisting of OH, COOH, and COORa, wherein Ra is C₁₋₁₀ alkyl;
R₂ and R₄ is independently selected from hydrogen or C₁₋₁₀ alkyl;
R₃ is selected from C₁₋₁₀ alkyl or halogen; and n is selected from 1 to 5.

In an embodiment, the present disclosure provides a method for preventing HIV subtypes causing disease in a subject in need thereof comprising: administering to a HIV high risk subject a relevant dose of a compound selected from the group consisting of Formula III

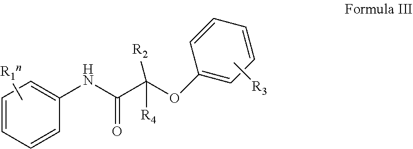

or its stereoisomers, pharmaceutically acceptable salts, polymorphs, solvates and hydrates thereof, wherein,
R₁ is independently selected from the group consisting of OH, COOH, and COORa, wherein Ra is C₁₋₅ alkyl;
R₂ and R₄ is independently selected from hydrogen or C₁₋₅ alkyl;
R₃ is selected from C₁₋₅ alkyl or chlorine; and n is selected from 1 to 2.

In an embodiment, the present disclosure provides a method for treating or preventing HIV infection in a subject by immune evasion mediated by internalization of CD80/86 receptors by its Nef protein comprising: administering to a subject a relevant dose of a compound selected from the group consisting of Formula I,

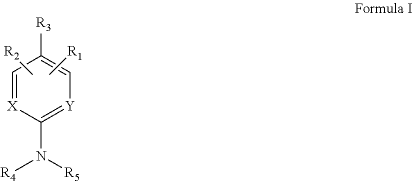

or its stereoisomers, pharmaceutically acceptable salts, polymorphs, solvates and hydrates thereof, wherein
R₁ and R₂ are independently selected from the group consisting of hydrogen, C₆₋₁₀ aryl, C₁₋₁₀ alkyl, C₂₋₁₀ alkenyl, C₂₋₁₀ alkynyl, and amino, wherein C₁₋₁₀ alkyl, C₂₋₁₀ alkenyl, C₂₋₁₀ alkynyl, and C₆₋₁₀ aryl are optionally substituted with one to four substituents independently selected from hydroxyl, halogen, C₁₋₁₀ alkyl, C₁₋₁₀ alkoxy or C₂₋₁₀ heterocyclyl;
R₃ is C₆₋₁₀ aryl, wherein C₆₋₁₀ aryl is optionally substituted with one to four substituents independently selected from C₁₋₁₀ alkoxy, nitro, halogen or C₁₋₁₀ alkyl, wherein C₁₋₁₀ alkyl, and C₁₋₁₀ alkoxy is optionally substituted with one to four substituents selected from halogen, hydrogen, hydroxyl, C₁₋₁₀ alkyl, C₁₋₁₀ alkoxy or C₂₋₁₀ heterocyclyl;
R₄ and R₅ are independently selected from the group consisting of hydrogen, C₁₋₁₀ alkyl, and —N═CHC₆ aryl, wherein —N═CHC₆ aryl is further substituted with one to four halogen;
or R₄ and R₅ are joined to form a C₂₋₁₀ heterocyclyl or a C₂₋₁₀ heteroaryl, wherein C₂₋₁₀ heterocyclyl or C₂₋₁₀ heteroaryl is optionally substituted with one to four substituents independently selected from C₁₋₁₀ alkyl; and
X and Y are N;

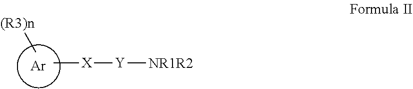

or its stereoisomers, pharmaceutically acceptable salts, polymorphs, solvates and hydrates thereof, wherein,
R₁, and R₂ are independently selected from the group consisting of hydrogen, C₁₋₁₀ alkyl, hydroxy, thio, amino, C₂₋₁₀ heterocyclyl, C₅₋₁₀ aryl, C₂₋₁₀ heteroaryl, acyl, amido, imido, sulfinyl, sulfonyl, carboxaldehyde, cyano, isocyano, azido, hydrazino, nitro, and halo; or
R₁ and R₂ are joined to form an optionally substituted monocyclic or a bicyclic ring, wherein C₁₋₁₀ alkyl, C₂₋₁₀ heterocyclyl, C₅₋₁₀ aryl, C₂₋₁₀ heteroaryl or monocyclic or a bicyclic ring are optionally substituted with one four substituents independently selected from hydroxy, C₁₋₁₀ alkyl, C₅₋₁₀ aryl, C₁₋₁₀ alkoxy, halogen, haloalkyl, perhaloalkyl, cyano, thio, and —CH₂-Ph-NH—COO-Ph-Me;
X is O;
Y is C═O;
Ar is selected from C₁₋₁₀ alkyl, C₅₋₁₀ aryl or C₂₋₁₀ heteroaryl;
R₃ is absent or is selected from hydroxy, C₁₋₁₀ alkyl, C₅₋₁₀ aryl, C₂₋₁₀ heteroaryl, C₂₋₁₀ heterocyclyl, C₁₋₁₀ alkoxy, halogen, haloalkyl, perhaloalkyl, nitro, cyano, CH₂PhNHCOOPh-Me, NHCONHPh-(Cl)₂ or CONHNHSO₂Ph-Me; and
n is selected from 0-5; and

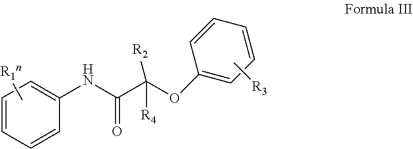

or its stereoisomers, pharmaceutically acceptable salts, polymorphs, solvates and hydrates thereof, wherein,
R₁ is independently selected from the group consisting of OH, COOH, and COORa, wherein Ra is C₁₋₁₀ alkyl;
R₂ and R₄ is independently selected from hydrogen or C₁₋₁₀ alkyl;
R₃ is selected from C₁₋₁₀ alkyl or halogen; and
n is selected from 1 to 5.

In an embodiment, the present disclosure provides a method for preventing HIV subtypes causing disease in a subject in need thereof comprising: administering to a HIV high risk subject a relevant dose of a compound selected from the group consisting of Formula I,

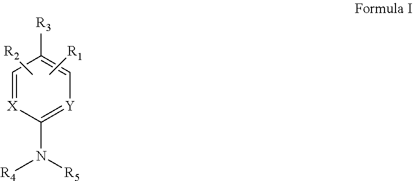

or its stereoisomers, pharmaceutically acceptable salts, polymorphs, solvates and hydrates thereof, wherein
R₁ and R₂ are independently selected from the group consisting of hydrogen, C₆₋₁₀ aryl, C₁₋₁₀ alkyl, and amino, wherein C₁₋₁₀ alkyl, and C₆₋₁₀ aryl are optionally substituted with one to four substituents independently selected from hydroxyl, halogen, C₁₋₁₀ alkyl, C₁₋₁₀ alkoxy or C₂₋₁₀ heterocyclyl;
R₃ is C₆₋₁₀ aryl, wherein C₆₋₁₀ aryl is optionally substituted with one to four substituents independently selected from C₁₋₁₀ alkoxy, nitro, halogen or C₁₋₁₀ alkyl, wherein C₁₋₁₀ alkyl and C₁₋₁₀ alkoxy is optionally substituted with one to four substituents selected from halogen, hydrogen, hydroxyl, C₁₋₁₀ alkyl, C₁₋₁₀ alkoxy or C₂₋₁₀ heterocyclyl;
R₄ and R₅ are independently selected from the group consisting of hydrogen, C₁₋₁₀ alkyl, and —N═CHC₆ aryl, wherein —N═CHC₆ aryl is further substituted with one to four halogen;
or R₄ and R₅ are joined to form a C₂₋₁₀ heterocyclyl or a C₂₋₁₀ heteroaryl, wherein C₂₋₁₀ heterocyclyl or C₂₋₁₀ heteroaryl is optionally substituted with one to four substituents independently selected from C₁₋₁₀ alkyl; and X and Y are N.

In an embodiment, the present disclosure provides a method of treating or preventing HIV infection in a subject by immune evasion mediated by internalization of CD80/86 receptors by its Nef protein comprising: administering to a subject a relevant dose of a compound selected from the group consisting of Formula I,

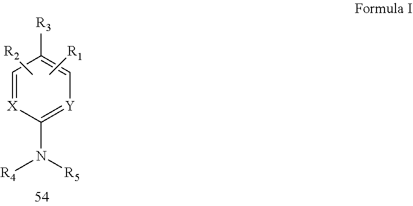

or its stereoisomers, pharmaceutically acceptable salts, polymorphs, solvates and hydrates thereof, wherein
R₁ and R₂ are independently selected from the group consisting of hydrogen, C₆₋₁₀ aryl, and C₁₋₁₀ alkyl, wherein C₁₋₁₀ alkyl, and C₆₋₁₀ aryl are optionally substituted with one to four substituents independently selected from hydroxyl, halogen, C₁₋₁₀ alkyl or C₁₋₁₀ alkoxy;
R₃ is C₆₋₁₀ aryl, wherein C₆₋₁₀ aryl is optionally substituted with one to four substituents independently selected from C₁₋₁₀ alkoxy, nitro, halogen or C₁₋₁₀ alkyl, wherein C₁₋₁₀ alkyl and C₁₋₁₀ alkoxy is optionally substituted with one to four substituents selected from halogen, hydrogen, hydroxyl, C₁₋₁₀ alkyl, C₁₋₁₀ alkoxy or C₂₋₁₀ heterocyclyl;
R₄ and R₅ are independently selected from the group consisting of hydrogen, C₁₋₁₀ alkyl, and —N═CHC₆ aryl, wherein —N═CHC₆ aryl is further substituted with one to four halogen;
or R₄ and R₅ are joined to form a C₂₋₁₀ heterocyclyl or a C₂₋₁₀ heteroaryl, wherein C₂₋₁₀ heterocyclyl or C₂₋₁₀ heteroaryl is optionally substituted with one to four substituents independently selected from C₁₋₁₀ alkyl; and X and Y are N.

In an embodiment, the present disclosure provides a method of treating or preventing HIV infection in a subject by immune evasion mediated by internalization of CD80/86 receptors by its Nef protein comprising: administering to a subject a relevant dose of a compound selected from the group consisting of Formula I,

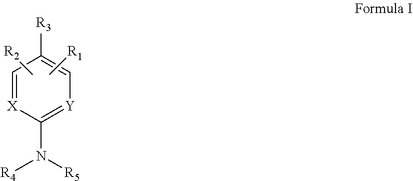

or its stereoisomers, pharmaceutically acceptable salts, polymorphs, solvates and hydrates thereof, wherein
R₁ and R₂ are independently selected from the group consisting of hydrogen, C₆ aryl, and C₁₋₅ alkyl, wherein C₁₋₅ alkyl, and C₆ aryl are optionally substituted with one to four substituents independently selected from hydroxyl, fluorine, C₁₋₅ alkyl or C₁₋₅ alkoxy;
R₃ is C₆ aryl, wherein C₆ aryl is optionally substituted with one to four substituents independently selected from C₁₋₅ alkoxy, nitro, halogen or C₁₋₅ alkyl, wherein C₁₋₅ alkyl and C₁₋₅ alkoxy is optionally substituted with one to four substituents selected from halogen, hydrogen, hydroxyl, C₁₋₅ alkyl, C₁₋₅ alkoxy or C₂₋₁₀heterocyclyl;
R₄ and R₅ are independently selected from the group consisting of hydrogen, C₁₋₅ alkyl, and —N═CHC₆ aryl, wherein —N═CHC₆ aryl is further substituted with one to four halogen;
or R₄ and R₅ are joined to form a C₂₋₁₀ heterocyclyl or a C₂₋₁₀ heteroaryl, wherein C₂₋₁₀ heterocyclyl or C₂₋₁₀ heteroaryl is optionally substituted with one to four substituents independently selected from C₁₋₅ alkyl; and X and Y are N.

In an embodiment, the present disclosure provides a method of treating or preventing HIV infection in a subject by immune evasion mediated by internalization of CD80/86 receptors by its Nef protein comprising: administering to a subject a relevant dose of a compound selected from the group consisting of Formula I,

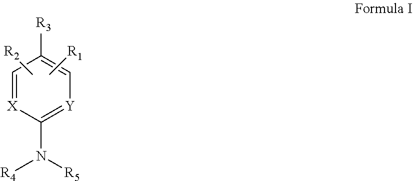

or its stereoisomers, pharmaceutically acceptable salts, polymorphs, solvates and hydrates thereof, wherein
R₁ and R₂ are independently selected from the group consisting of hydrogen, C₆ aryl, and CF₃, wherein C₆ aryl are optionally substituted with one to four substituents independently selected from hydroxyl, C₁₋₅ alkyl or C₁₋₅ alkoxy;
R₃ is C₆ aryl, wherein C₆ aryl is optionally substituted with one to four substituents independently selected from —OCH₃, —OCF₃, nitro, fluorine or CF₃;
R₄ and R₅ are independently selected from the group consisting of hydrogen, C₁₋₅ alkyl, and —N═CHC₆ aryl, wherein —N═CHC₆ aryl is further substituted with one to four substituents selected from fluorine and bromine; or R₄ and R₅ are joined to form a C₂₋₁₀ heterocyclyl or a C₂₋₁₀ heteroaryl, wherein C₂₋₁₀ heterocyclyl or C₂₋₁₀ heteroaryl is optionally substituted with one to four substituents independently selected from C₁₋₅ alkyl; and X and Y are N.

In an embodiment, the present disclosure provides a method of treating or preventing HIV infection in a subject by immune evasion mediated by internalization of CD80/86 receptors by its Nef protein comprising: administering to a subject a relevant dose of a compound selected from the group consisting of Formula I,

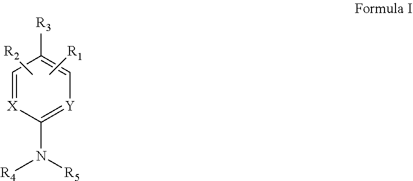

or its stereoisomers, pharmaceutically acceptable salts, polymorphs, solvates and hydrates thereof, wherein
R₁ and R₂ are independently selected from the group consisting of hydrogen, C₆₋₁₀ aryl, C₁₋₁₀ alkyl, C₂₋₁₀ alkenyl, C₂₋₁₀ alkynyl, and amino, wherein C₁₋₁₀ alkyl, C₂₋₁₀ alkenyl, C₂₋₁₀ alkynyl, and C₆₋₁₀ aryl are optionally substituted with one to four substituents independently selected from hydroxyl, halogen, C₁₋₁₀ alkyl, C₁₋₁₀ alkoxy or C₂₋₁₀ heterocyclyl;
R₃ is C₆₋₁₀ aryl, wherein C₆₋₁₀ aryl is optionally substituted with one to four substituents independently selected from C₁₋₁₀ alkoxy, nitro or C₁₋₁₀ alkyl, wherein C₁₋₁₀ alkyl and C₁₋₁₀ alkoxy is optionally substituted with one to four substituents selected from halogen, hydrogen, hydroxyl, C₁₋₁₀ alkyl, C₁₋₁₀ alkoxy or C₂₋₁₀ heterocyclyl;
R₄ and R₅ are independently selected from the group consisting of hydrogen, C₁₋₁₀ alkyl, and —N═CHC₆ aryl, wherein —N═CHC₆ aryl is further substituted with one to four halogen;
or R₄ and R₅ are joined to form a C₂₋₁₀ heterocyclyl or a C₂₋₁₀ heteroaryl, wherein C₂₋₁₀ heterocyclyl or C₂₋₁₀ heteroaryl is optionally substituted with one to four substituents independently selected from C₁₋₁₀ alkyl; and X and Y are N.

In an embodiment, the present disclosure provides a method of treating or preventing HIV infection in a subject by immune evasion mediated by internalization of CD80/86 receptors by its Nef protein comprising: administering to a subject a relevant dose of a compound selected from the group consisting of Formula II

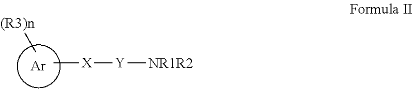

or its stereoisomers, pharmaceutically acceptable salts, polymorphs, solvates and hydrates thereof, wherein,
R₁, and R₂ are independently selected from the group consisting of hydrogen, C₁₋₁₀ alkyl, hydroxy, thio, amino, C₂₋₁₀ heterocyclyl, C₅₋₁₀ aryl, C₂₋₁₀ heteroaryl, acyl, amido, imido, sulfinyl, sulfonyl, carboxaldehyde, cyano, isocyano, azido, hydrazino, nitro, and halo; or
R₁ and R₂ are joined to form an optionally substituted monocyclic or a bicyclic ring, wherein C₁₋₁₀ alkyl, C₂₋₁₀ heterocyclyl, C₅₋₁₀ aryl, C₂₋₁₀ heteroaryl or monocyclic or a bicyclic ring are optionally substituted with one four substituents independently selected from hydroxy, C₁₋₁₀ alkyl, C₅₋₁₀ aryl, C₁₋₁₀ alkoxy, halogen, haloalkyl, perhaloalkyl, cyano, thio, and —CH₂-Ph-NH—COO-Ph-Me;
X is O;
Y is C═O;
Ar is selected from C₁₋₁₀ alkyl, C₅₋₁₀ aryl or C₂₋₁₀ heteroaryl;
R₃ is absent or is selected from hydroxy, C₁₋₁₀ alkyl, C₅₋₁₀ aryl, C₂₋₁₀ heteroaryl, C₂₋₁₀ heterocyclyl, C₁₋₁₀ alkoxy, halogen, haloalkyl, perhaloalkyl, nitro, cyano, CH₂PhNHCOOPh-Me, NHCONHPh-(Cl)₂ or CONHNHSO₂Ph-Me; and n is selected from 0-5.

In an embodiment, the present disclosure provides a method of treating or preventing HIV infection in a subject by immune evasion mediated by internalization of CD80/86 receptors by its Nef protein comprising: administering to a subject a relevant dose of a compound selected from the group consisting of Formula II

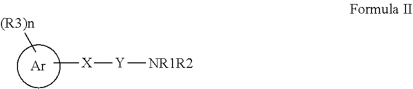

or its stereoisomers, pharmaceutically acceptable salts, polymorphs, solvates and hydrates thereof, wherein,
R₁, and R₂ are independently selected from the group consisting of hydrogen, C₁₋₁₀ alkyl, hydroxy, thio, amino, C₂₋₁₀ heterocyclyl, C₅₋₁₀ aryl, C₂₋₁₀ heteroaryl, acyl, amido, imido, sulfinyl, sulfonyl, carboxaldehyde, cyano, isocyano, azido, hydrazino, nitro, and halo; or
R₁ and R₂ are joined to form an optionally substituted monocyclic or a bicyclic ring, wherein C₁₋₁₀ alkyl, C₂₋₁₀ heterocyclyl, C₅₋₁₀ aryl, C₂₋₁₀ heteroaryl or monocyclic or a bicyclic ring are optionally substituted with one four substituents independently selected from hydroxy, C₁₋₁₀ alkyl, C₅₋₁₀ aryl, C₁₋₁₀ alkoxy, halogen, haloalkyl, perhaloalkyl, cyano, thio, and —CH₂-Ph-NH—COO-Ph-Me;
X is O;
Y is C═O;
Ar is selected from C₁₋₁₀ alkyl, C₅₋₁₀ aryl or C₂₋₁₀ heteroaryl;
R₃ is absent or is selected from hydroxy, C₁₋₁₀ alkyl, C₅₋₁₀ aryl, C₂₋₁₀ heteroaryl, C₂₋₁₀ heterocyclyl, C₁₋₁₀ alkoxy, halogen, haloalkyl, perhaloalkyl, nitro, cyano, CH₂PhNHCOOPh-Me, NHCONHPh-(Cl)₂ or CONHNHSO₂Ph-Me; and n is selected from 0-5.

In an embodiment, the present disclosure provides a method of treating or preventing HIV infection in a subject by immune evasion mediated by internalization of CD80/86 receptors by its Nef protein comprising: administering to a subject a relevant dose of a compound selected from the group consisting of Formula II

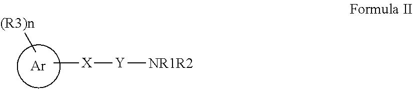

or its stereoisomers, pharmaceutically acceptable salts, polymorphs, solvates and hydrates thereof, wherein,
R₁, and R₂ are independently selected from the group consisting of hydrogen, C₁₋₁₀ alkyl, hydroxy, thio, amino, C₂₋₁₀ heterocyclyl, C₅₋₁₀ aryl, C₂₋₁₀ heteroaryl, acyl, amido, imido, sulfinyl, sulfonyl, carboxaldehyde, cyano, isocyano, azido, hydrazino, nitro, and halo; or
R₁ and R₂ are joined to form an optionally substituted monocyclic or a bicyclic ring, wherein C₁₋₁₀ alkyl, C₂₋₁₀ heterocyclyl, C₅₋₁₀ aryl, C₂₋₁₀ heteroaryl or monocyclic or a bicyclic ring are optionally substituted with one four substituents independently selected from hydroxy, C₁₋₁₀ alkyl, C₅₋₁₀ aryl, C₁₋₁₀ alkoxy, halogen, haloalkyl, perhaloalkyl, cyano, thio, and —CH₂-Ph-NH—COO-Ph-Me;
X is O;
Y is C═O;
Ar is selected from C₅₋₁₀ aryl or C₂₋₁₀ heteroaryl;
R₃ is absent or is selected from hydroxy, C₁₋₁₀ alkyl, C₅₋₁₀ aryl, C₂₋₁₀ heteroaryl, C₂₋₁₀ heterocyclyl, C₁₋₁₀ alkoxy, halogen, haloalkyl, perhaloalkyl, nitro, cyano, CH₂PhNHCOOPh-Me, NHCONHPh-(Cl)₂ or CONHNHSO₂Ph-Me; and n is selected from 0-5.

In an embodiment, the present disclosure provides a method of treating or preventing HIV infection in a subject by immune evasion mediated by internalization of CD80/86 receptors by its Nef protein comprising: administering to a subject a relevant dose of a compound selected from the group consisting of Formula II

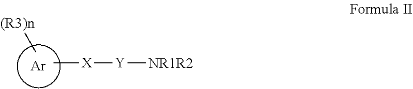

or its stereoisomers, pharmaceutically acceptable salts, polymorphs, solvates and hydrates thereof, wherein,
R₁, and R₂ are independently selected from the group consisting of hydrogen, C₁₋₁₀ alkyl, hydroxy, thio, amino, C₂₋₁₀ heterocyclyl, C₅₋₁₀ aryl, C₂₋₁₀ heteroaryl, acyl, amido, imido, sulfinyl, sulfonyl, carboxaldehyde, cyano, isocyano, azido, hydrazino, nitro, and halo; or
R₁ and R₂ are joined to form an optionally substituted monocyclic or a bicyclic ring, wherein C₁₋₁₀ alkyl, C₂₋₁₀ heterocyclyl, C₅₋₁₀ aryl, C₂₋₁₀ heteroaryl or monocyclic or a bicyclic ring are optionally substituted with one four substituents independently selected from hydroxy, C₁₋₁₀ alkyl, C₅₋₁₀ aryl, C₁₋₁₀ alkoxy, halogen, haloalkyl, perhaloalkyl, cyano, thio, and —CH₂-Ph-NH—COO-Ph-Me;
X is O;
Y is C═O;
Ar is C₅₋₁₀ aryl;
R₃ is absent or is selected from hydroxy, C₁₋₁₀ alkyl, C₅₋₁₀ aryl, C₂₋₁₀ heteroaryl, C₂₋₁₀ heterocyclyl, C₁₋₁₀ alkoxy, halogen, haloalkyl, perhaloalkyl, nitro, cyano, CH₂PhNHCOOPh-Me, NHCONHPh-(Cl)₂ or CONHNHSO₂Ph-Me; and n is selected from 0-5.

In an embodiment, the present disclosure provides a method of treating or preventing HIV infection in a subject by immune evasion mediated by of CD80/86 receptors by its Nef protein comprising: administering to a subject a relevant dose of a compound selected from the group consisting of Formula III

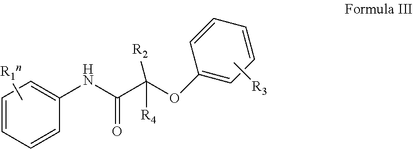

or its stereoisomers, pharmaceutically acceptable salts, polymorphs, solvates and hydrates thereof, wherein,
R₁ is independently selected from the group consisting of OH, COOH, and COORa, wherein Ra is C₁₋₁₀ alkyl;
R₂ and R₄ is independently selected from hydrogen or C₁₋₁₀ alkyl;
R₃ is selected from C₁₋₁₀ alkyl or halogen; and n is selected from 1 to 5.

In an embodiment, the present disclosure provides a method of treating or preventing HIV infection in a subject by immune evasion mediated by internalization of CD80/86 receptors by its Nef protein comprising: administering to a subject a relevant dose of a compound selected from the group consisting of Formula III

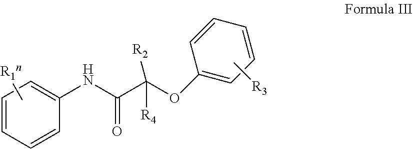

or its stereoisomers, pharmaceutically acceptable salts, polymorphs, solvates and hydrates thereof, wherein,
R₁ is independently selected from the group consisting of OH, COOH, and COORa, wherein Ra is C₁₋₅ alkyl;
R₂ and R₄ is independently selected from hydrogen or C₁₋₅ alkyl;
R₃ is selected from C₁₋₅ alkyl or chlorine; and n is selected from 1 to 2.

In an embodiment, the present disclosure provides a compound of Formula I, II, or III or its or its stereoisomers, pharmaceutically acceptable salts, polymorphs, solvates and hydrates thereof, which is selected from a group consisting of
2-(3,5-Dimethyl-1H-pyrazol-1-yl)-5-(4-nitrophenyl)pyrimidine (I₁a),
2-(3,5-Dimethyl-1H-pyrazol-1-yl)-5-[4-(trifluoromethyl)phenyl]pyrimidine (I₁b),
2-(3,5-Dimethyl-1H-pyrazol-1-yl)-5-[4-(trifluoromethoxy)phenyl]pyrimidine (I₁c),
2-(3,5-Dimethyl-1H-pyrazol-1-yl)-5-(4-methoxyphenyl)pyrimidine (I₁d),
2-(3,5-Dimethyl-1H-pyrazol-1-yl)-5-(3-nitrophenyl)pyrimidine (I₁e),
2-(3,5-Dimethyl-1H-pyrazol-1-yl)-5-(2-nitrophenyl)pyrimidine (I₁f),
2-[2-(4-Bromobenzylidene)hydrazinyl)-5-(4-nitrophenyl)]pyrimidine (I₂a),
2-(2-(4-Fluorobenzylidene)hydrazinyl)-5-(4-nitrophenyl)pyrimidine (I₂b),
(E)-2-(2-(4-Fluorobenzylidene)hydrazinyl)-5-(4-(trifluoromethyl)phenyl)pyrimidine (I₂c),
(E)-2-[2-(4-Bromobenzylidene)hydrazinyl)-5-(4-(trifluoromethoxy)phenyl]pyrimidine (I₂d),
(E)-2-[2-(4-Fluorobenzylidene)hydrazinyl)-5-(4-methoxyphenyl]pyrimidine (I₂e),
(E)-2-(2-(4-Fluorobenzylidene)hydrazinyl)-5-(3-nitrophenyl)pyrimidine (I₂f),
(E)-2-[(2-(4-Bromobenzylidene)hydrazinyl)-5-(2-nitrophenyl)]pyrimidine (I₂g),
N,N-Dimethyl-5-phenylpyrimidin-2-amine (I₃a),
4-(5-(4-(Trifluoromethyl)phenyl)pyrimidin-2-yl)morpholine (I₃b),
5-(4-Fluorophenyl)-N,N-dimethylpyrimidin-2-amine (I₃c),
4-(5-(4-Fluorophenyl)pyrimidin-2-yl)morpholine (I₃d),
2-(2-amino-5-(4-methoxyphenyl)-6-(trifluoromethyl)pyrimidin-4-yl)-4-ethyl-5-methoxyphenol (I₄₎,
Phenyl 2-thioxobenzo[d]oxazole-3(2H)-carboxylate (II₁),
p-Tolyl (3,4-dichlorophenyl)carbamate (II₂a),
[1,1′-biphenyl]-4-yl (3,4-dichlorophenyl)carbamate (II₂b),
4-Methoxyphenyl diphenylcarbamate (II₂c),
Di-p-tolyl [methylenebis(4,1-phenylene)]dicarbamate (II₂d),
Naphthalen-2-yl (2-fluorophenyl)carbamate (II₃),
2-[3-(3,4-dichlorophenyl)ureido]-4-nitrophenyl (3,4-dichlorophenyl)carbamate (II₄),
3-[(1,3,4-oxadiazol-2-yl)phenyl] diphenylcarbamate (II₅),
2,4-di-tert-butyl-6-methoxyphenyl phenylcarbamate (II₆),
2-[(2-Tosylhydrazine-1-carbonyl)phenyl] phenylcarbamate (II₇),
Ethyl 4-[2-(4-ethylphenoxy)acetamido]benzoate (III₁a),
Methyl 4-[2-(2-isopropylphenoxy)acetamido]benzoate (III₁b),
Propyl 4-[2-(4-isopropylphenoxy)acetamido]benzoate (III₁c),
Isopropyl 4-[2-(4-isopropylphenoxy)acetamido]benzoate (III₁d),
Propyl 4-[2-(m-tolyloxy)acetamido]benzoate (III₁e),
Isobutyl 2-hydroxy-4-[2-(4-isopropylphenoxy)acetamido]benzoate (III₁f),
4-[2-(3,5-dimethylphenoxy)acetamido]-2-hydroxybenzoic acid (III₂a),
4-(2-Phenoxypropanamido)benzoic acid (III₂b),
Ethyl 4-[2-(4-chlorophenoxy)-2-methylpropanamido]benzoate (III₂c), and
Methyl 4-[2-(o-tolyloxy)propanamido]benzoate (III₂d).

The compounds for Formula I, II, or III causes restoration of immune signaling via T cell activation through inhibiting Nef-CD80/86 interactions.

In an embodiment, the present disclosure provides a method of treating or preventing HIV subtypes causing disease in a subject comprising: administering to a HIV infected subject a therapeutic dose of a compound selected the group consisting of Formula I, wherein compound of Formula I is selected from a group consisting of:

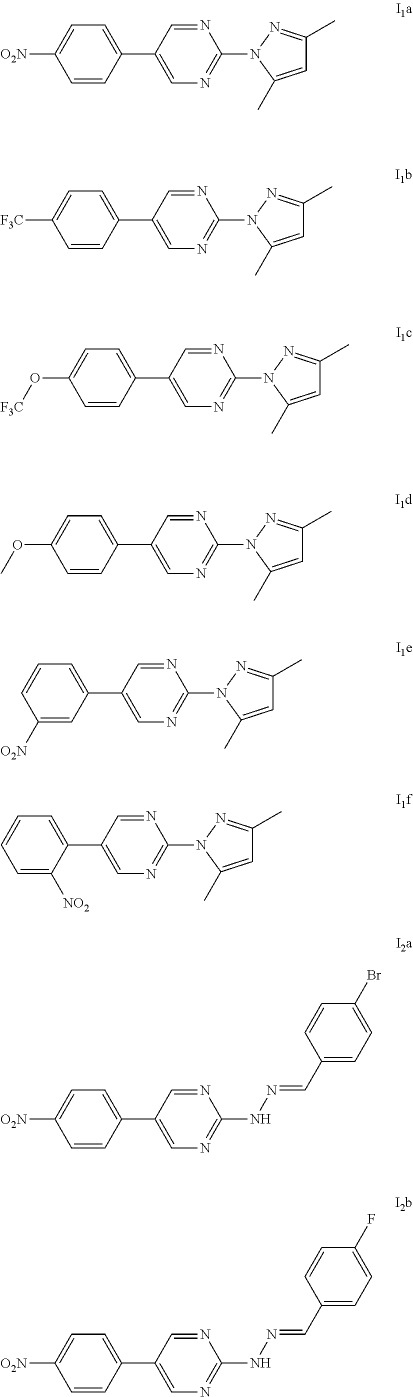

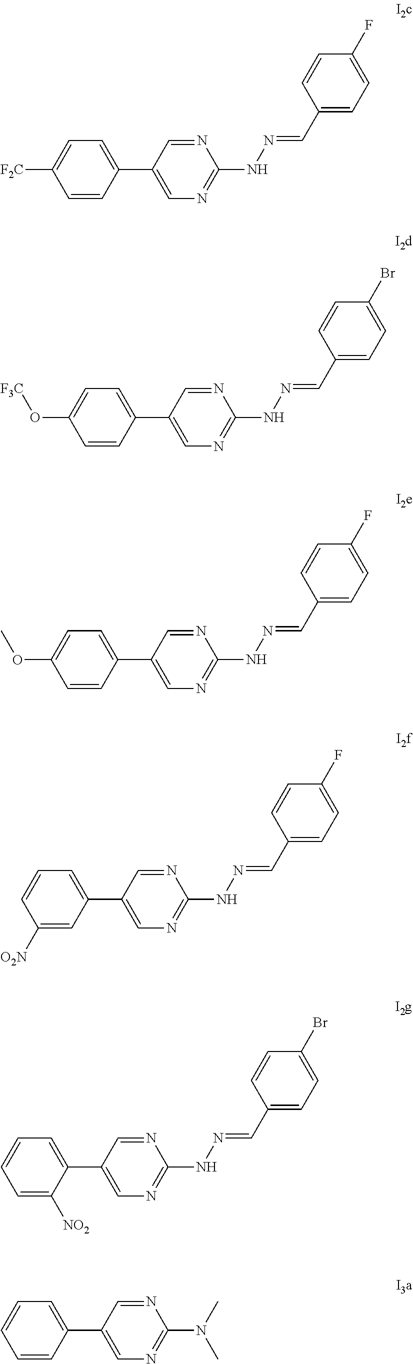

In an embodiment, the present disclosure provides a method of treating or preventing HIV subtypes causing disease in a subject in need thereof comprising: administering to a HIV high risk subject a relevant dose of a compound selected from the group consisting of Formula I, wherein compound of Formula I is selected from a group consisting of:

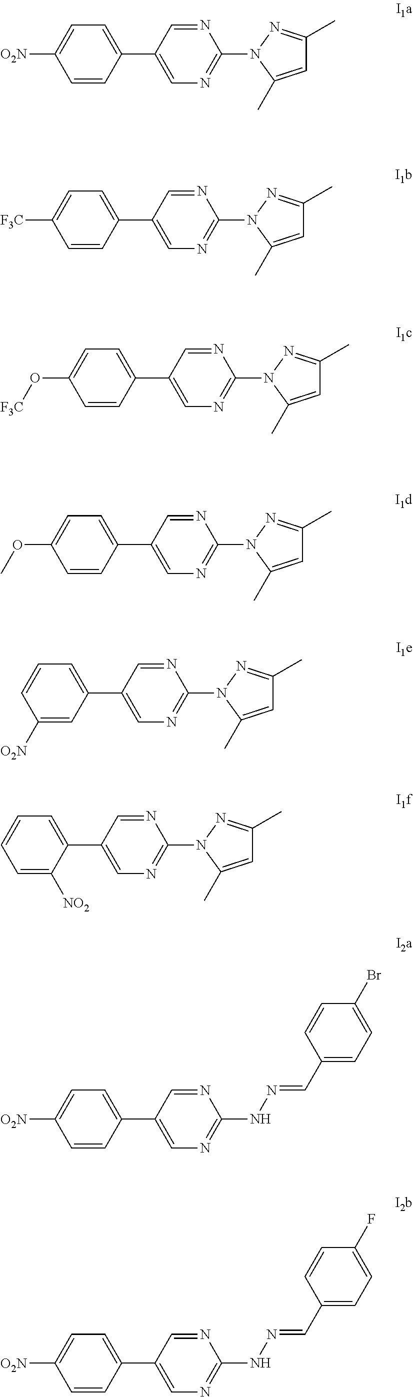

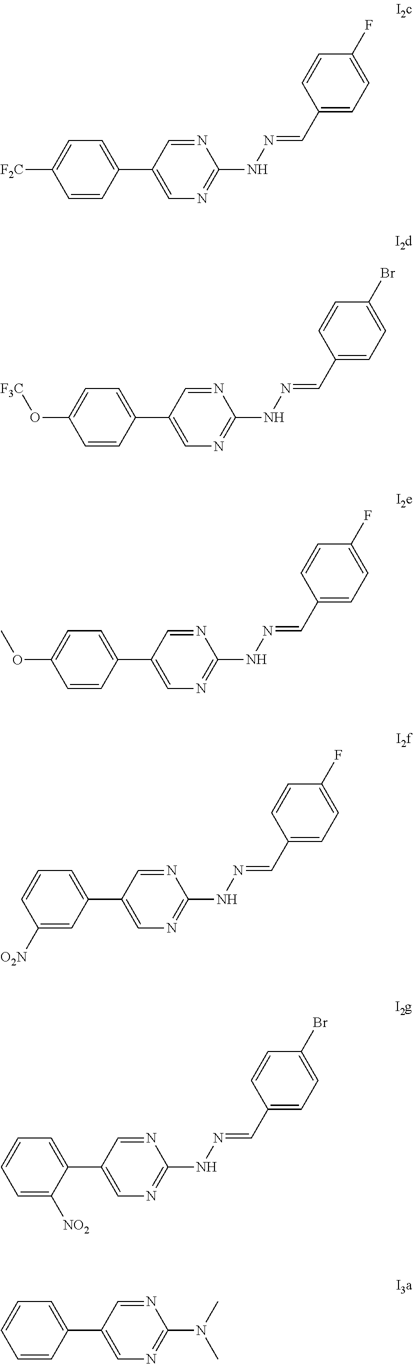

In an embodiment, the present disclosure provides a method of treating or preventing HIV subtypes causing disease in a subject comprising: administering to a HIV infected subject a therapeutic dose of a compound selected the group consisting of Formula I, wherein compound of Formula I is selected from a group consisting of:

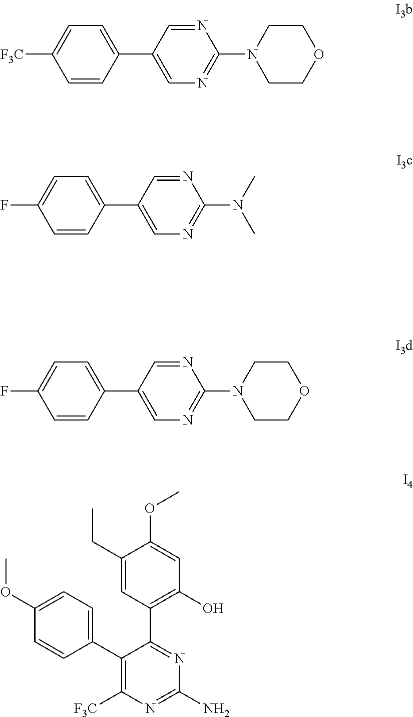 [SMILES: FC(F)(F)c1ccc(-c2cnc(N3CCOCC3)nc2)cc1 ; CN(C)c1ncc(-c2ccc(F)cc2)cn1 ; Fc1ccc(-c2cnc(N3CCOCC3)nc2)cc1 ; CCc1cc(-c2nc(N)nc(C(F)(F)F)c2-c2ccc(OC)cc2)c(O)cc1OC]

In an embodiment, the present disclosure provides a method of treating or preventing HIV subtypes causing disease in a subject in need thereof comprising: administering to a HIV high risk subject a relevant dose of a compound selected from the group consisting of Formula I, wherein compound of Formula I is selected from a group consisting of:

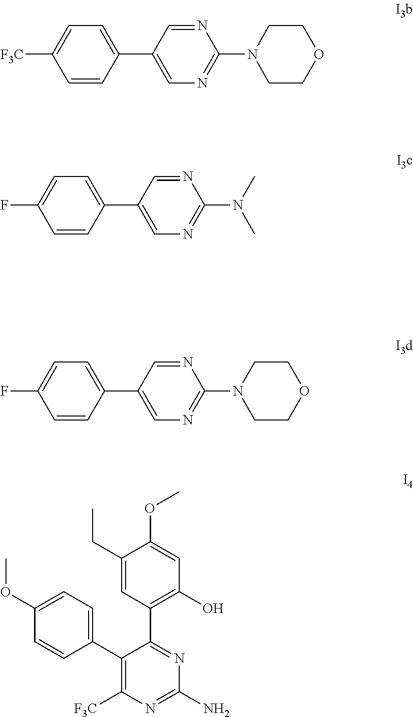 [SMILES: FC(F)(F)c1ccc(-c2cnc(N3CCOCC3)nc2)cc1 ; CN(C)c1ncc(-c2ccc(F)cc2)cn1 ; Fc1ccc(-c2cnc(N3CCOCC3)nc2)cc1 ; CCc1cc(-c2nc(N)nc(C(F)(F)F)c2-c2ccc(OC)cc2)c(O)cc1OC]

In an embodiment, the present disclosure provides a method of treating or preventing HIV subtypes causing disease in a subject comprising: administering to a HIV infected subject a therapeutic dose of a compound selected the group consisting of Formula I, wherein compound of Formula I is selected from a group consisting of:

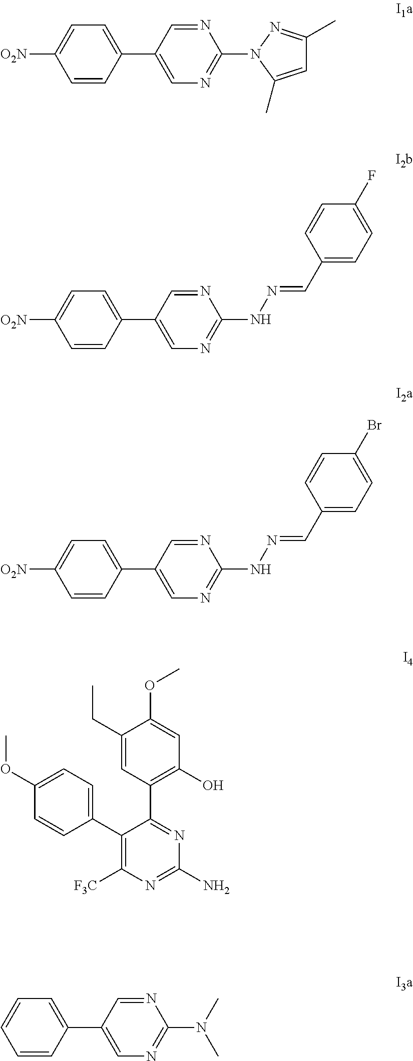 [SMILES: Cc1cc(C)n(-c2ncc(-c3ccc([N+](=O)[O-])cc3)cn2)n1 ; O=[N+]([O-])c1ccc(-c2cnc(N/N=C/c3ccc(F)cc3)nc2)cc1 ; O=[N+]([O-])c1ccc(-c2cnc(N/N=C/c3ccc(Br)cc3)nc2)cc1 ; CCc1cc(-c2nc(N)nc(C(F)(F)F)c2-c2ccc(OC)cc2)c(O)cc1OC ; CN(C)c1ncc(-c2ccccc2)cn1]

In an embodiment, the present disclosure provides a method of treating or preventing HIV subtypes causing disease in a subject in need thereof comprising: administering to a HIV high risk subject a relevant dose of a compound selected from the group consisting of Formula I, wherein compound of Formula I is selected from a group consisting of:

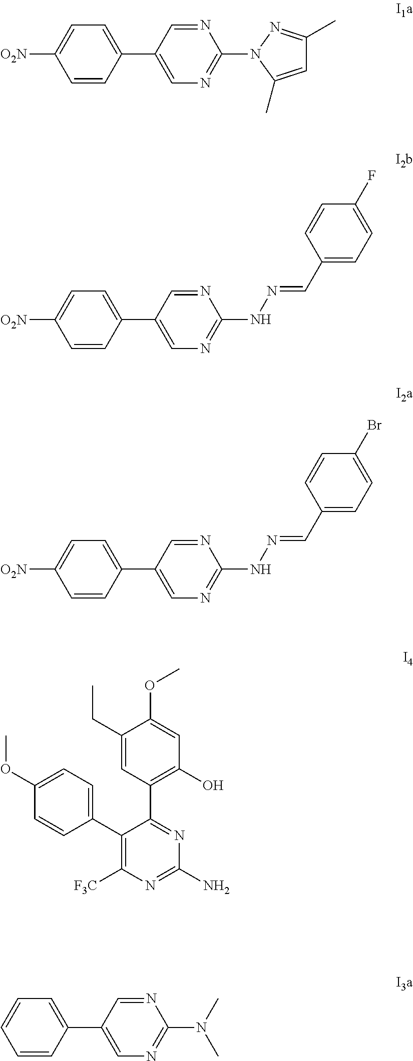 [SMILES: Cc1cc(C)n(-c2ncc(-c3ccc([N+](=O)[O-])cc3)cn2)n1 ; O=[N+]([O-])c1ccc(-c2cnc(N/N=C/c3ccc(F)cc3)nc2)cc1 ; O=[N+]([O-])c1ccc(-c2cnc(N/N=C/c3ccc(Br)cc3)nc2)cc1 ; CCc1cc(-c2nc(N)nc(C(F)(F)F)c2-c2ccc(OC)cc2)c(O)cc1OC ; CN(C)c1ncc(-c2ccccc2)cn1]

In an embodiment, the present disclosure provides a method of treating or preventing HIV subtypes causing disease in a subject comprising: administering to a HIV infected subject a therapeutic dose of a compound selected the group consisting of Formula II, wherein compound of Formula II is selected from a group consisting of:

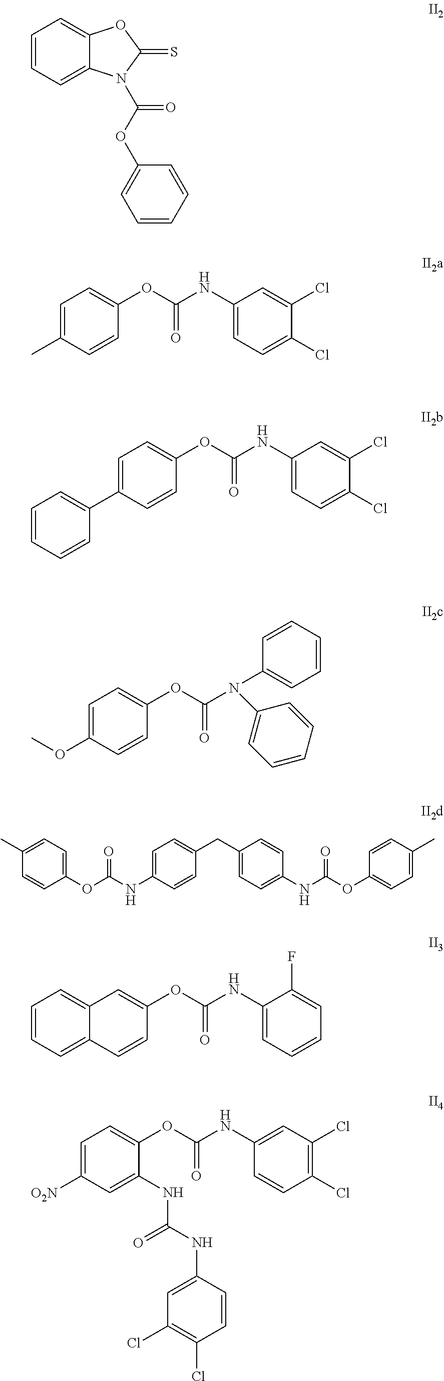

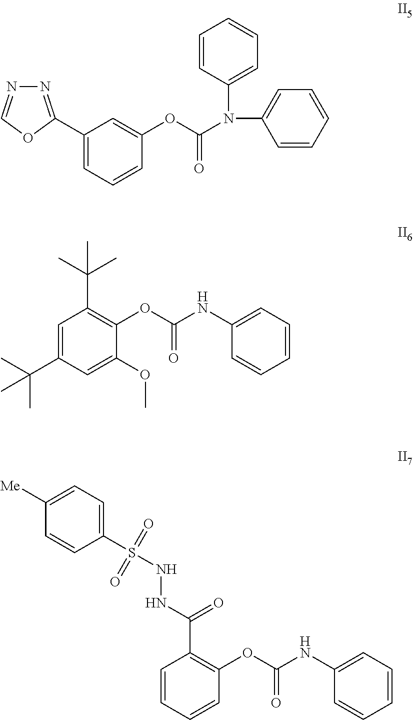 [SMILES: O=C(Oc1cccc(-c2nnco2)c1)N(c1ccccc1)c1ccccc1 ; COc1cc(C(C)(C)C)cc(C(C)(C)C)c1OC(=O)Nc1ccccc1 ; Cc1ccc(S(=O)(=O)NNC(=O)c2ccccc2OC(=O)Nc2ccccc2)cc1]

In an embodiment, the present disclosure provides a method of treating or preventing HIV subtypes causing disease in a subject in need thereof comprising: administering to a HIV high risk subject a relevant dose of a compound selected from the group consisting of Formula II, wherein compound of Formula II is selected from a group consisting of:

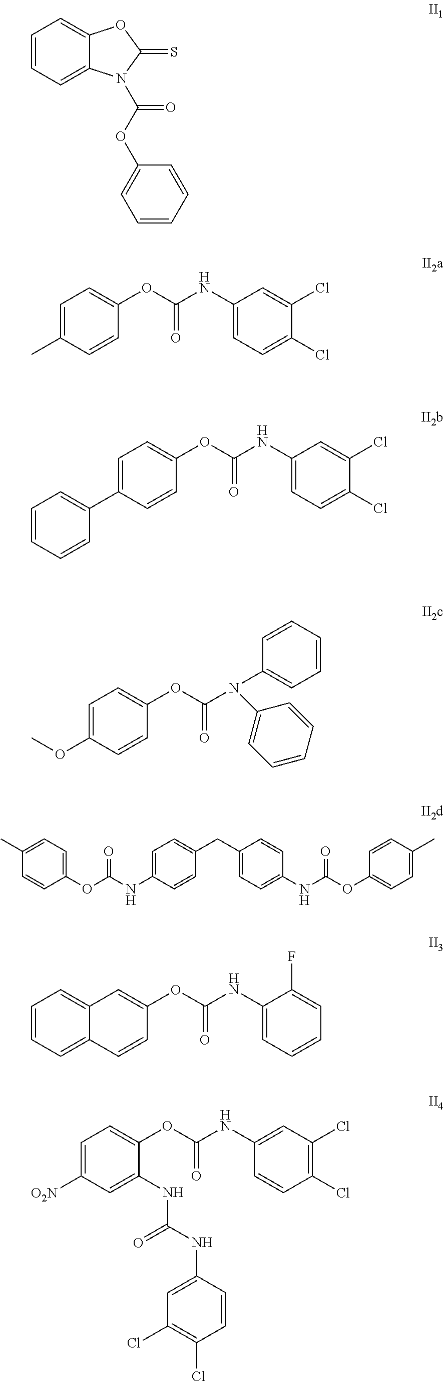

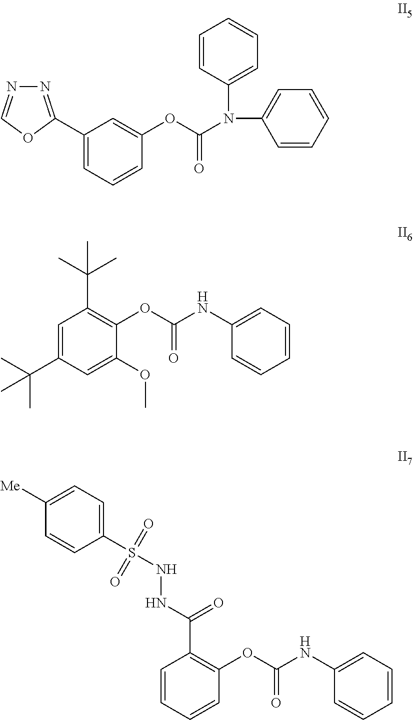 [SMILES: O=C(Oc1cccc(-c2nnco2)c1)N(c1ccccc1)c1ccccc1 ; COc1cc(C(C)(C)C)cc(C(C)(C)C)c1OC(=O)Nc1ccccc1 ; Cc1ccc(S(=O)(=O)NNC(=O)c2ccccc2OC(=O)Nc2ccccc2)cc1]

In an embodiment, the present disclosure provides a method of treating or preventing HIV subtypes causing disease in a subject comprising: administering to a HIV infected subject a therapeutic dose of a compound selected the group consisting of Formula III, wherein compound of Formula III is selected from a group consisting of:

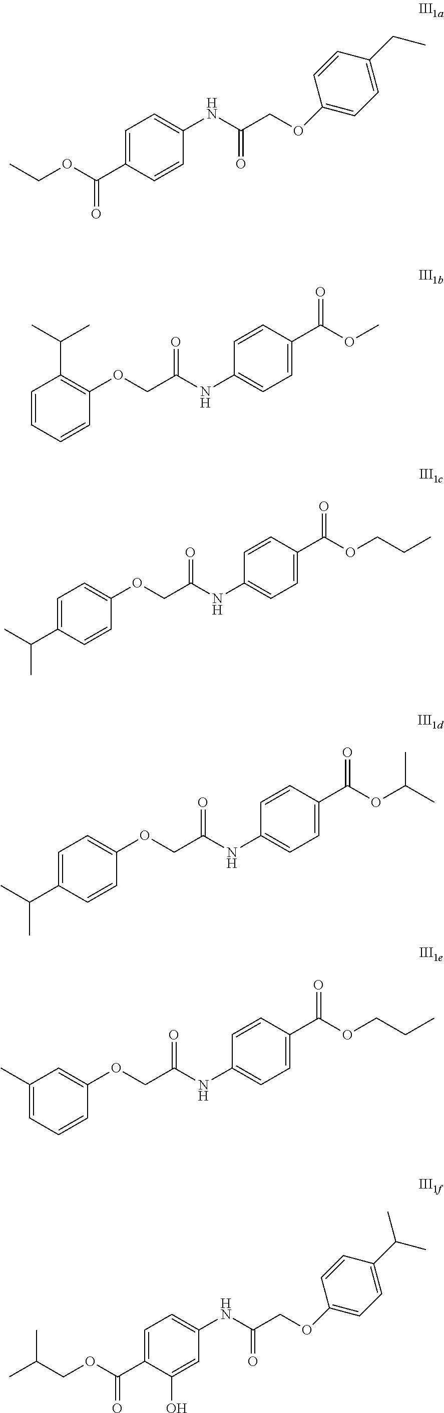

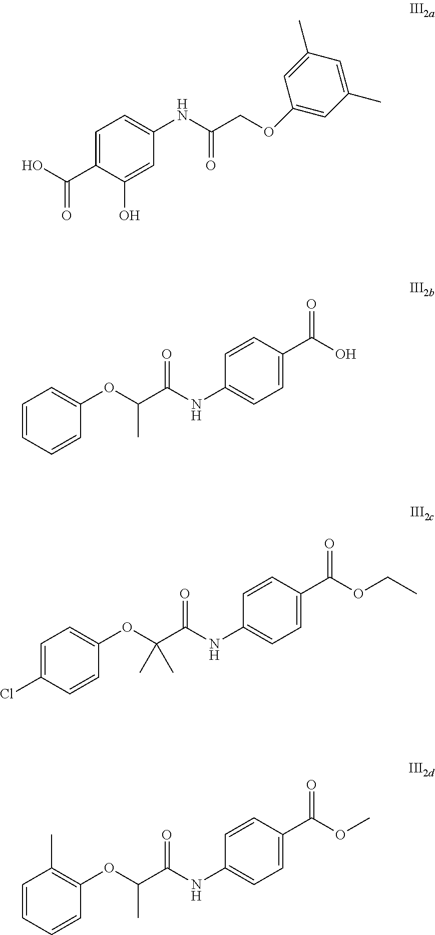 [SMILES: Cc1cc(C)cc(OCC(=O)Nc2ccc(C(=O)O)c(O)c2)c1 ; CC(Oc1ccccc1)C(=O)Nc1ccc(C(=O)O)cc1 ; CCOC(=O)c1ccc(NC(=O)C(C)(C)Oc2ccc(Cl)cc2)cc1 ; COC(=O)c1ccc(NC(=O)C(C)Oc2ccccc2C)cc1]

In an embodiment, the present disclosure provides a method of treating or preventing HIV subtypes causing disease in a subject in need thereof comprising: administering to a HIV high risk subject a relevant dose of a compound selected from the group consisting of Formula III, wherein compound of Formula III is selected from a group consisting of:

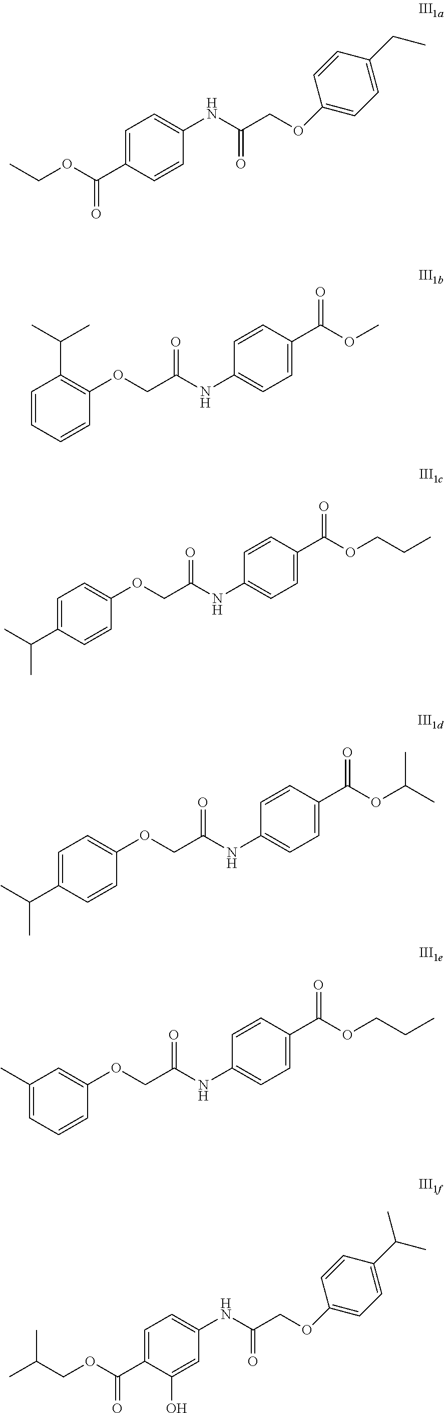

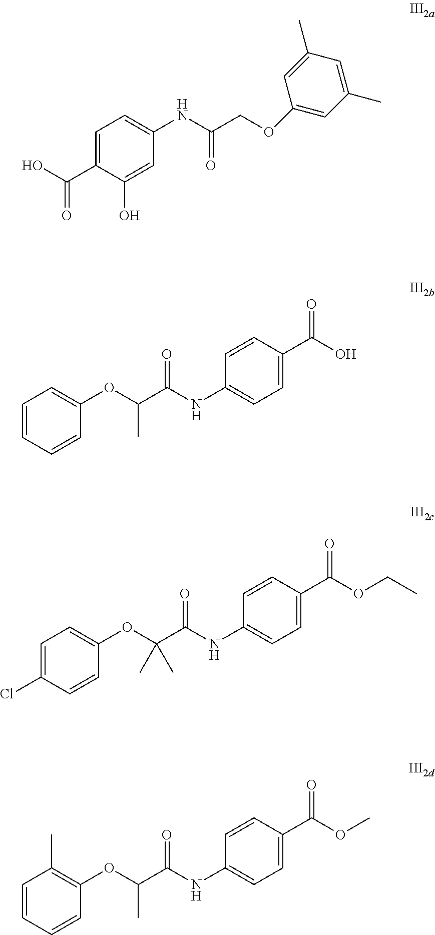 [SMILES: Cc1cc(C)cc(OCC(=O)Nc2ccc(C(=O)O)c(O)c2)c1 ; CC(Oc1ccccc1)C(=O)Nc1ccc(C(=O)O)cc1 ; CCOC(=O)c1ccc(NC(=O)C(C)(C)Oc2ccc(Cl)cc2)cc1 ; COC(=O)c1ccc(NC(=O)C(C)Oc2ccccc2C)cc1]

In an embodiment, the present disclosure provides a method of treating or preventing HIV infection in a subject by immune evasion mediated by internalization of CD80/86 receptors by its Nef protein comprising: administering to a subject a relevant dose of a compound selected from the group consisting of Formula I,

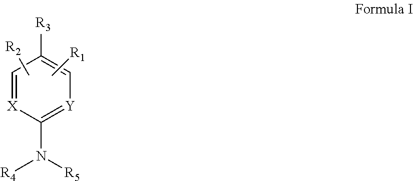

or its stereoisomers, pharmaceutically acceptable salts, polymorphs, solvates and hydrates thereof, wherein
R₁ and R₂ are independently selected from the group consisting of hydrogen, C₆₋₁₀ aryl, C₁₋₁₀ alkyl, C₂₋₁₀ alkenyl, C₂₋₁₀ alkynyl, and amino, wherein C₁₋₁₀ alkyl, C₂₋₁₀ alkenyl, C₂₋₁₀ alkynyl, and C₅₋₁₀ aryl are optionally substituted with one to four substituents independently selected from hydroxyl, halogen, C₁₋₁₀ alkyl, C₁₋₁₀ alkoxy, and C₂₋₁₀ heterocyclyl;
R₃ is C₆₋₁₀ aryl, wherein C₆₋₁₀ aryl is optionally substituted with one to four substituents independently selected from C₁₋₁₀ alkoxy, nitro, halogen or C₁₋₁₀ alkyl, wherein C₁₋₁₀ alkyl, and C₁₋₁₀ alkoxy is optionally substituted with one to four substituents selected from halogen, hydrogen, hydroxyl, C₁₋₁₀ alkyl, C₁₋₁₀ alkoxy or C₂₋₁₀ heterocyclyl;
R₄ and R₅ are independently selected from the group consisting of hydrogen, C₁₋₁₀ alkyl, and —N═CHC₆ aryl, wherein —N═CHC₆ aryl is further substituted with one to four halogen;
or R₄ and R₅ are joined to form a C₂₋₁₀ heterocyclyl or a C₂₋₁₀ heteroaryl, wherein C₂₋₁₀ heterocyclyl or C₂₋₁₀ heteroaryl is optionally substituted with one to four substituents independently selected from C₁₋₁₀ alkyl; and
X and Y are N;

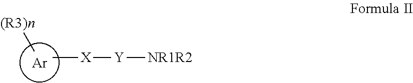

or its stereoisomers, pharmaceutically acceptable salts, polymorphs, solvates and hydrates thereof, wherein,
R₁, and R₂ are independently selected from the group consisting of hydrogen, C₁₋₁₀ alkyl, hydroxy, thio, amino, C₂₋₁₀ heterocyclyl, C₅₋₁₀ aryl, C₂₋₁₀ heteroaryl, acyl, amido, imido, sulfinyl, sulfonyl, carboxaldehyde, cyano, isocyano, azido, hydrazino, nitro, and halo; or
R₁ and R₂ are joined to form an optionally substituted monocyclic or a bicyclic ring, wherein C₁₋₁₀ alkyl, C₂₋₁₀ heterocyclyl, C₅₋₁₀ aryl, C₂₋₁₀ heteroaryl or monocyclic or a bicyclic ring are optionally substituted with one four substituents independently selected from hydroxy, C₁₋₁₀ alkyl, C₅₋₁₀ aryl, C₁₋₁₀ alkoxy, halogen, haloalkyl, perhaloalkyl, cyano, thio, and —CH₂-Ph-NH—COO-Ph-Me;
X is selected from O;
Y is C═O;
Ar is selected from C₁₋₁₀ alkyl, C₅₋₁₀ aryl or C₂₋₁₀ heteroaryl;
R₃ is absent or is selected from hydroxy, C₁₋₁₀ alkyl, C₅₋₁₀ aryl, C₂₋₁₀ heteroaryl, C₂₋₁₀ heterocyclyl, C₁₋₁₀ alkoxy, halogen, haloalkyl, perhaloalkyl, nitro, cyano, CH₂PhNHCOOPh-Me, NHCONHPh-(Cl)₂ or CONHNHSO₂Ph-Me; and
n is selected from 0-5; and

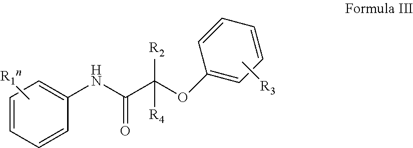

or its stereoisomers, pharmaceutically acceptable salts, polymorphs, solvates and hydrates thereof, wherein,
R₁ is independently selected from the group consisting of OH, COOH, and COORa, wherein Ra is C₁₋₁₀ alkyl;
R₂ and R₄ is independently selected from hydrogen or C₁₋₁₀ alkyl;
R₃ is selected from C₁₋₁₀ alkyl or halogen; and
n is selected from 1 to 5. In another embodiment of the present disclosure, the relevant dose is in the range of 50 to 1000 μM.

In an embodiment, the present disclosure provides a method of treating or preventing or treating HIV infection in a subject by immune evasion as described herein, wherein the compounds causes restoration of immune signaling via T cell activation through inhibiting Nef-CD80/86 interactions.

In an embodiment, the present disclosure provides a method for treating/preventing infections which have low levels of CD80/86 receptors compared to non-infected state, selected from the including chronic lymphocytic leukemia, colon carcinoma, multiple myeloma, viral infections including HIV, HPV, herpes.

In an embodiment, the present disclosure relates to a method for preventing HIV subtypes causing disease in a subject in need thereof comprising: administering to a HIV high risk subject a relevant dose of a compound selected from the group consisting of 4-(5-(4-(trifluoromethyl)phenyl)pyrimidin-2-yl)morpholine (I₃b), naphthalen-2-yl (2-fluorophenyl)carbamate (II₃), and 4-[2-(3,5-dimethylphenoxy)acetamido]-2-hydroxybenzoic acid (III₂a). In another embodiment of the present disclosure, the relevant dose is in the range of 50 to 1000 μM.

In an embodiment, the present disclosure relates to a method for preventing HIV subtypes causing disease in a subject comprising: administering to a HIV infected subject a therapeutic dose of a compound selected from a group consisting of 4-(5-(4-(trifluoromethyl)phenyl)pyrimidin-2-yl)morpholine (I₃b), naphthalen-2-yl (2-fluorophenyl)carbamate (II₃), 4-[2-(3,5-dimethylphenoxy)acetamido]-2-hydroxybenzoic acid (III₂a). and combinations thereof. In another embodiment of the present disclosure, the therapeutic dose is in the range of 50 to 1000 μM. In yet another embodiment, the compound is a combination of 4-(5-(4-(trifluoromethyl)phenyl)pyrimidin-2-yl)morpholine (I₃b), naphthalen-2-yl (2-fluorophenyl)carbamate (II₃), 4-[2-(3,5-dimethylphenoxy)acetamido]-2-hydroxybenzoic acid (III₂a).

In an embodiment, the present disclosure relates to method for treating HIV subtypes causing disease in a subject comprising: administering to a HIV infected subject a therapeutic dose of a compound selected from a group consisting of 4-(5-(4-(trifluoromethyl)phenyl)pyrimidin-2-yl)morpholine (I₃b), naphthalen-2-yl (2-fluorophenyl)carbamate (II₃), and 4-[2-(3,5-dimethylphenoxy)acetamido]-2-hydroxybenzoic acid (III₂a). In another embodiment of the present disclosure, the relevant dose is in the range of 50 to 1000 μM.

In an embodiment, the present disclosure relates to method for treating HIV subtypes causing disease in a subject comprising: administering to a HIV infected subject a therapeutic dose of a compound selected from a group consisting of 4-(5-(4-(trifluoromethyl)phenyl)pyrimidin-2-yl)morpholine (I₃b), naphthalen-2-yl (2-fluorophenyl)carbamate (II₃), 4-[2-(3,5-dimethylphenoxy)acetamido]-2-hydroxybenzoic acid (III₂a), and combinations thereof. In another embodiment of the present disclosure, the therapeutic dose is in the range of 50 to 1000 μM. In yet another embodiment, the compound is a combination of 4-(5-(4-(trifluoromethyl)phenyl)pyrimidin-2-yl)morpholine (I₃b), naphthalen-2-yl (2-fluorophenyl)carbamate (II₃), 4-[2-(3,5-dimethylphenoxy)acetamido]-2-hydroxybenzoic acid (III₂a).

In an embodiment, the present disclosure relates to a method for preventing or treating HIV infection in a subject by immune evasion mediated by internalization of CD80/86 receptors by its Nef protein comprising: administering to a subject a relevant dose of a compound selected from 4-(5-(4-(trifluoromethyl)phenyl)pyrimidin-2-yl)morpholine (I₃b), naphthalen-2-yl (2-fluorophenyl)carbamate (II₃), or 4-[2-(3,5-dimethylphenoxy)acetamido]-2-hydroxybenzoic acid (III₂a). In another embodiment of the present disclosure, the relevant dose is in the range of 50 to 1000 μM.

In an embodiment, the present disclosure relates to a method for treating/preventing infections which have low levels of CD80/86 receptors compared to non-infected state, selected from the including cancers like chronic lymphocytic leukemia, colon carcinoma, multiple myeloma, viral infections including HIV, HPV, herpes using the compound of Formula I, Formula II, and Formula III or its stereoisomers, pharmaceutically acceptable salts, complexes, hydrates, solvates, tautomers, polymorphs, racemic mixtures, optically active forms and pharmaceutically active derivative thereof.

The infections in bovine, feline, simian that act through Nef superfamily pathways.

In an embodiment, the present disclosure relates to a compound of Formula I, Formula II, and Formula III or its stereoisomers, pharmaceutically acceptable salts, complexes, hydrates, solvates, tautomers, polymorphs, racemic mixtures, optically active forms and pharmaceutically active derivative thereof, for use in killing or inhibiting the growth virus.

In an embodiment, the present disclosure relates to a compound of Formula I, Formula II, and Formula III or its stereoisomers, pharmaceutically acceptable salts, complexes, hydrates, solvates, tautomers, polymorphs, racemic mixtures, optically active forms and pharmaceutically active derivative thereof, for use in killing or inhibiting the growth of HIV.

In an embodiment, the present disclosure relates to use of a compound of Formula I, Formula II, and Formula III or its stereoisomers, pharmaceutically acceptable salts, complexes, hydrates, solvates, tautomers, polymorphs, racemic mixtures, optically active forms and pharmaceutically active derivative thereof, in killing or inhibiting the growth of HIV.

In an embodiment, the present disclosure relates to a compound of Formula I, Formula II, and Formula III or its stereoisomers, pharmaceutically acceptable salts, complexes, hydrates, solvates, tautomers, polymorphs, racemic mixtures, optically active forms and pharmaceutically active derivative thereof, for use in treating a disease or condition in a patient wherein said disease or condition is caused by HIV.

In an embodiment, the present disclosure relates to use of a compound of Formula I, Formula II, and Formula III or its stereoisomers, pharmaceutically acceptable salts, complexes, hydrates, solvates, tautomers, polymorphs, racemic mixtures, optically active forms and pharmaceutically active derivative thereof, in treating disease or condition in a patient, wherein said disease or condition is caused by HIV. The patient is a typically a mammal, preferably a human.

In an embodiment, the present disclosure relates to a method of treating a disease or condition in a patent, said method comprising administering to a patient a compound of Formula I, Formula II, and Formula III or its stereoisomers, pharmaceutically acceptable salts, complexes, hydrates, solvates, tautomers, polymorphs, racemic mixtures, optically active forms and pharmaceutically active derivative thereof, wherein said disease or condition is caused by HIV.

In an embodiment, the present disclosure relates to a compound of Formula I, Formula II, and Formula III or its stereoisomers, pharmaceutically acceptable salts, complexes, hydrates, solvates, tautomers, polymorphs, racemic mixtures, optically active forms and pharmaceutically active derivative thereof, for use as a medicament.

In an embodiment, the present disclosure relates to a compound of Formula I, Formula II, and Formula III or its stereoisomers, pharmaceutically acceptable salts, complexes, hydrates, solvates, tautomers, polymorphs, racemic mixtures, optically active forms and pharmaceutically active derivative thereof, for use in the preparation of medicaments for inhibiting viral growth.

In an embodiment, the present disclosure relates to medicaments that include a compound of Formula I, Formula II, and Formula III, or an addition salt of the compound of Formula I, Formula II, and Formula III with a pharmaceutically acceptable acid or base. These medicaments find their use in therapeutics, especially in the treatment of viral infection caused by HIV.

In an embodiment, the present disclosure relates to the use of a compound of Formula I, Formula II, and Formula III or its stereoisomers, pharmaceutically acceptable salts, complexes, hydrates, solvates, tautomers, polymorphs, racemic mixtures, optically active forms and pharmaceutically active derivative thereof, in the manufacture of a medicament for the production of an antiviral effect in a warm-blooded animal such as man.

In an embodiment, the present disclosure relates to a method for producing an antiviral effect in a warm-blooded animal such as man, said method including administering to said animal an effective amount of a compound of Formula I, Formula II, and Formula III or a pharmaceutically acceptable salt thereof.

In an embodiment, the present disclosure relates to a compound of Formula I, Formula II, and Formula III or a pharmaceutically acceptable salt thereof, for use in the treatment and/or prophylaxis of viral infections in a warm-blooded animal, such as man.

In an embodiment, the present disclosure relates to a compound of Formula I, Formula II, and Formula III or a pharmaceutically acceptable salt thereof, for the therapeutic and prophylactic treatment of mammals including humans, in particular in treating viral infections caused by HIV, is normally formulated in accordance with standard pharmaceutical practice as a pharmaceutical composition.

In an embodiment, the present disclosure relates to a pharmaceutical composition including a compound of Formula I, Formula II, and Formula III or its stereoisomers, pharmaceutically acceptable salts, complexes, hydrates, solvates, tautomers, polymorphs, racemic mixtures, optically active forms and pharmaceutically active derivative thereof, and at least one pharmaceutically acceptable carrier, diluent, or excipient.

The term, “pharmaceutically acceptable” includes compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The compounds of Formula I, Formula II, and Formula III may form stable pharmaceutically acceptable acid or base salts, and in such cases administration of a compound as a salt may be appropriate. The salts may be formed by conventional means. The compositions of the disclosure may be in a form suitable for oral use, for topical use, for administration by inhalation, for administration by insufflation or for parenteral administration.

The compositions of the present disclosure may be obtained by conventional procedures using conventional pharmaceutical excipients well known in the art.

The compounds disclosed herein may be applied as a sole therapy or may involve, in addition to a compound of the disclosure, one or more other substances and/or treatments.

### Process and Characterization Data

There is also provided a process as shown in the following Schemes 1-7, for the preparation of compounds of the Formula I, Formula II, and Formula III, wherein all the groups are as defined earlier. The examples given below are provided by the way of illustration only and therefore should not be construed to limit the scope of the invention.

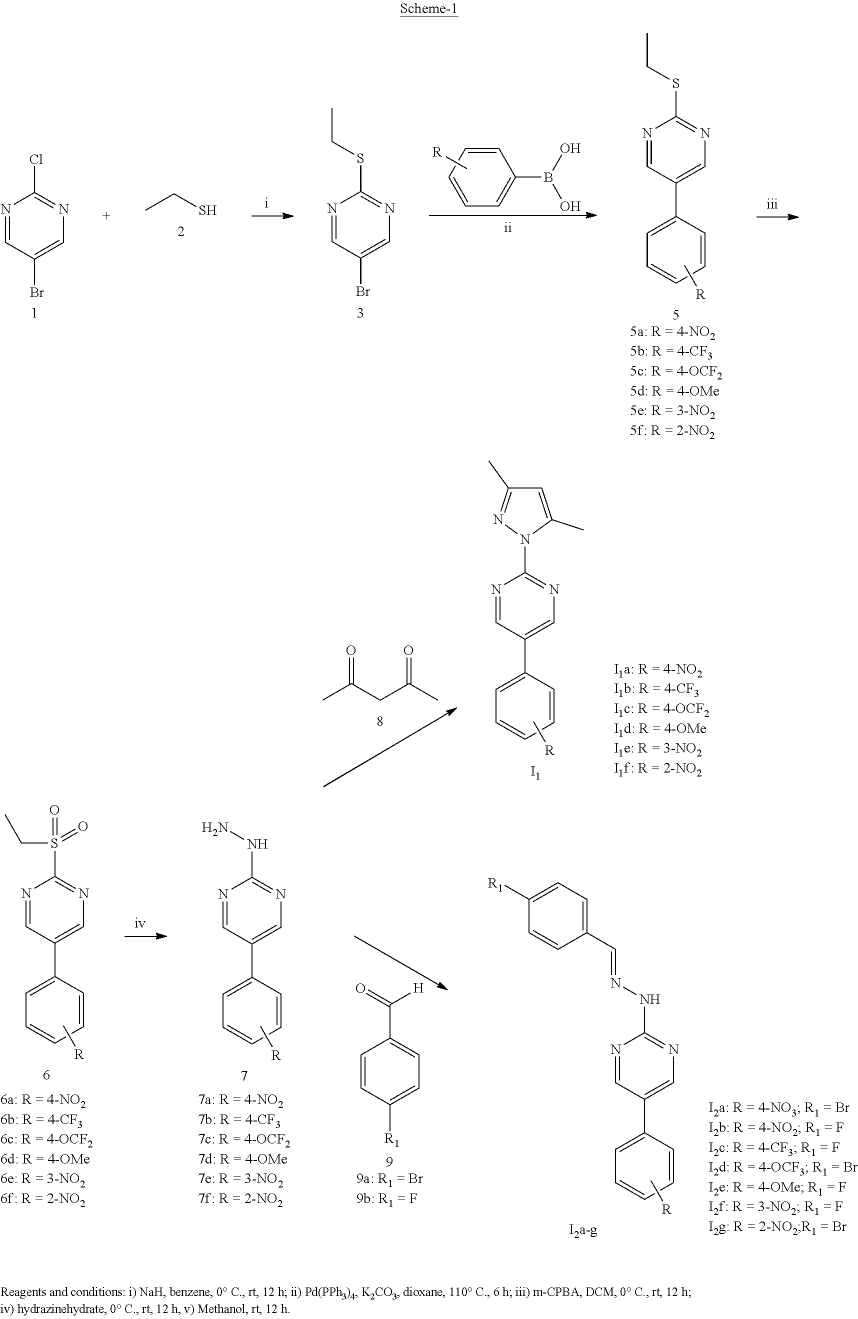

### General Procedure for the Synthesis of I_{1a-f} of Scheme-1

To a solution of compound 7 (3.4 mmol) in methanol was added acetyl acetone 8 (5.19 mmol) and catalytic amounts of acetic acid. The resulting mixture was stirred at 60° C.-70° C. for 5 h. After completion of the reaction (reaction monitored by TLC) the solvent was removed under vacuum, and the compound was extracted with EtOAc. The combined organic layer was dried over anhydrous Na₂SO₄ and concentrated under reduced pressure. The crude products were purified on a silica gel column using hexane/EtOAc to furnish compound I₁a-f.

### 2-(3,5-Dimethyl-1H-pyrazol-1-yl)-5-(4-nitrophenyl)pyrimidine (I₁a, Scheme 1)

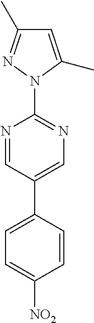

¹H NMR (400 MHz, CDCl₃) δ 9.28 (m, 2H), 8.38-8.24 (m, 2H), 7.94-7.80 (m, 2H), 6.14 (s, 1H), 2.38 (s, 6H); HRMS (ESI-TOF) calcd for C₁₅H₁₄N₅O₂ [M+H]⁺ 296.1147, found 296.1147.

### 2-(3,5-Dimethyl-1H-pyrazol-1-yl)-5-[4-(trifluoromethyl)phenyl]pyrimidine (I₁b, Scheme 1)

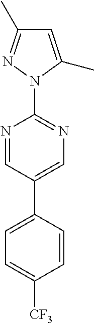

¹H NMR (500 MHz, CDCl₃) δ 9.24 (s, 2H), 7.70-7.67 (m, 2H), 7.61-7.58 (m, 2H), 6.13 (s, 1H), 2.36 (m, 6H); HRMS (ESI-TOF) calcd for C₁₆H₁₃F₃N₄ [M+H]⁺ 318.1092, found 318.1083.

### 2-(3,5-Dimethyl-1H-pyrazol-1-yl)-5-[4-(trifluoromethoxy)phenyl]pyrimidine (I₁c, Scheme 1)

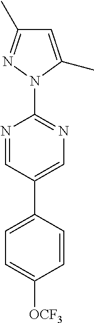

¹H NMR (500 MHz, CDCl₃) δ 9.36 (s, 2H), 7.68-7.48 (m, 2H), 7.19-6.96 (m, 2H), 6.12 (s, 1H), 2.44-2.25 (m, 6H); HRMS (ESI-TOF) calcd for C₁₆H₁₃F₃N₄O [M+H]⁺ 334.1041, found 334.1062.

### 2-(3,5-Dimethyl-1H-pyrazol-1-yl)-5-(4-methoxyphenyl)pyrimidine (I₁d, Scheme 1)

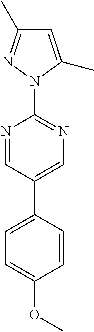

¹H NMR (500 MHz, CDCl₃) δ 9.21 (s, 2H), 7.63-7.55 (m, 2H), 7.12-6.96 (m, 2H), 6.12 (s, 1H), 3.81 (s, 3H), 2.36 (s, 6H); HRMS (ESI-TOF) calcd for C₁₆H₁₆N₄O [M+H]⁺ 280.1324, found 280.1348.

### 2-(3,5-Dimethyl-1H-pyrazol-1-yl)-5-(3-nitrophenyl)pyrimidine (I₁e, Scheme 1)

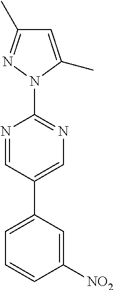

¹H NMR (500 MHz, CDCl₃) δ 9.26 (s, 2H), 8.56 (s, 1H), 8.22-8.19 (m, 1H), 7.99-7.91 (m, 1H), 7.68-7.59 (m, 1H), 6.13 (s, 1H), 2.37 (s, 6H); HRMS (ESI-TOF) calcd for C₁₅H₁₃N₅O₂ [M+H]⁺ 295.1069, found 295.1054.

### 2-(3,5-Dimethyl-1H-pyrazol-1-yl)-5-(2-nitrophenyl)pyrimidine (I₁f, Scheme 1)

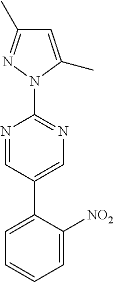

¹H NMR (500 MHz, CDCl₃) δ 9.12 (s, 2H), 8.29-8.27 (m, 1H), 7.82-7.79 (d, J=29.8 Hz, 1H), 7.60 (s, 1H), 6.13 (s, 1H), 2.36 (m, 6H); HRMS (ESI-TOF) calcd for C₁₅H₁₃N₅O₂ [M+H]⁺ 295.1069, found 295.1047.

### General Procedure for the Synthesis of I₂a-g of Scheme 1:

To a solution of compound 7 (0.4 mmol) in methanol/ethanol was added substituted benzaldehyde 9 (0.51 mmol) and catalytic amounts of acetic acid. The resulting mixture was stirred at room temperature for 5 h. After completion of the reaction (reaction monitored by TLC) the solvent was removed under vacuum, and the compound was extracted with EtOAc. The combined organic layer was dried over anhydrous Na₂SO₄ and concentrated under reduced pressure. The crude products were purified on a silica gel column using hexane/EtOAc to furnish compound I₂a-g.

### 2-[2-(4-Bromobenzylidene)hydrazinyl)-5-(4-nitrophenyl)]pyrimidine (I₂a, Scheme 1)

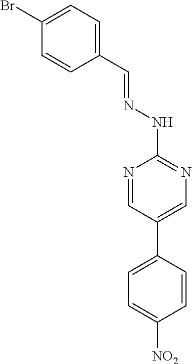 [SMILES: O=[N+]([O-])c1ccc(-c2cnc(N/N=C/c3ccc(Br)cc3)nc2)cc1]

¹H NMR (400 MHz, CDCl₃) δ 8.74 (s, 1H), 8.71 (s, 2H), 8.28 (d, J=8.8 Hz, 2H), 7.86 (s, 1H), 7.61 (dd, J=15.3, 8.6 Hz, 5H), 7.48 (d, J=8.5 Hz, 3H); HRMS (ESI-TOF) calcd for C₁₇H₁₃BrN₅O₂: [M+H] 399.2280 found 399.2282.

### 2-(2-(4-Fluorobenzylidene)hydrazinyl)-5-(4-nitrophenyl)pyrimidine (I₂b, Scheme 1)

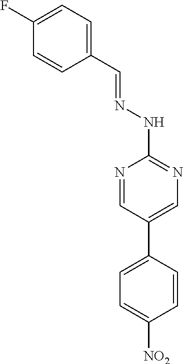 [SMILES: O=[N+]([O-])c1ccc(-c2cnc(N/N=C/c3ccc(F)cc3)nc2)cc1]

¹H NMR (400 MHz, CDCl₃) δ 8.74 (s, 1H), 8.71 (s, 2H), 8.28 (d, J=8.8 Hz, 2H), 7.86 (s, 1H), 7.61 (dd, J=15.3, 8.6 Hz, 5H), 7.48 (d, J=8.5 Hz, 3H); HRMS (ESI-TOF) calcd for C₁₇H₁₃FN₅O₂: [M+H] 338.1053 found 338.1055.

### (E)-2-(2-(4-Fluorobenzylidene)hydrazinyl)-5-(4-(trifluoromethyl)phenyl)pyrimidine (I₂c, Scheme 1)

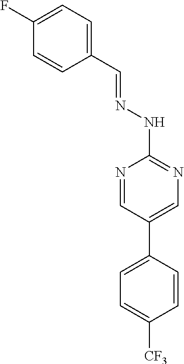 [SMILES: Fc1ccc(/C=N/Nc2ncc(-c3ccc(C(F)(F)F)cc3)cn2)cc1]

¹H NMR (400 MHz, CDCl₃) δ 9.03 (s, 1H), 8.86 (m, 2H), 7.69-7.64 (m, 2H), 7.63-7.58 (m, 2H), 7.56 (s, 1H), 7.54-7.48 (m, 2H), 7.30-7.23 (m, 2H); HRMS (ESI-TOF) calcd for C₁₈H₁₂F₄N₄: [M+H] 360.0998 found 360.0986.

### (E)-2-[2-(4-Bromobenzylidene)hydrazinyl)-5-(4-(trifluoromethoxy)phenyl]pyrimidine (I₂d, Scheme 1)

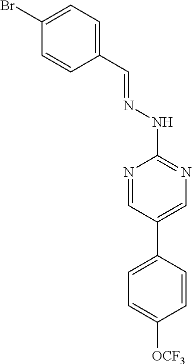 [SMILES: FC(F)(F)Oc1ccc(-c2cnc(N/N=C/c3ccc(Br)cc3)nc2)cc1]

¹H NMR (500 MHz, CDCl₃) δ 9.81 (s, 1H), 8.87 (s, 2H), 7.67-7.57 (m, 4H), 7.32-7.24 (m, 2H), 7.15-7.09 (m, 2H), 7.00 (s, 1H); HRMS (ESI-TOF) calcd for C₁₈H₁₂BrF₃N₄O: [M+H] 436.0147 found 436.0126.

### (E)-2-[2-(4-Fluorobenzylidene)hydrazinyl)-5-(4-methoxyphenyl]pyrimidine (I₂e, Scheme 1)

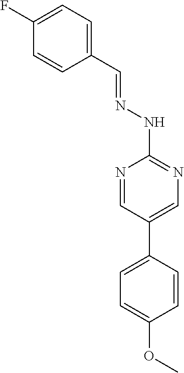 [SMILES: COc1ccc(-c2cnc(N/N=C/c3ccc(F)cc3)nc2)cc1]

¹H NMR (500 MHz, CDCl₃) δ 9.01 (s, 1H), 8.84-8.77 (m, 2H), 7.63-7.59 (m, 2H), 7.56 (s, 1H), 7.54-7.49 (m, 2H), 7.29-7.25 (m, 2H), 7.08-7.04 (m, 2H), 3.91-3.72 (m, 3H); HRMS (ESI-TOF) calcd for C₁₈H₁₅FN₄O: [M+H] 322.1230 found 322.1242.

### (E)-2-(2-(4-Fluorobenzylidene)hydrazinyl)-5-(3-nitrophenyl)pyrimidine (I₂f, Scheme 1)

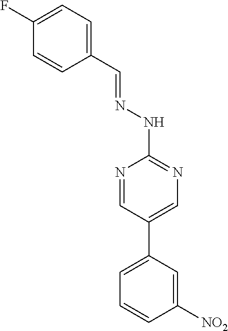 [SMILES: O=[N+]([O-])c1cccc(-c2cnc(N/N=C/c3ccc(F)cc3)nc2)c1]

¹H NMR (500 MHz, CDCl₃) δ 9.04 (s, 1H), 8.89-8.83 (m, 2H), 8.59 (s, 1H), 8.25 (s, 1H), 8.01 (s, 1H), 7.71 (s, 1H), 7.57 (s, 1H), 7.54-7.48 (m, 2H), 7.32-7.23 (m, 2H); HRMS (ESI-TOF) calcd for C₁₇H₁₂FN₅O₂: [M+H] 337.0975 found 337.0954.

### (E)-2-[(2-(4-Bromobenzylidene)hydrazinyl)-5-(2-nitrophenyl)]pyrimidine (I₂g, Scheme 1)

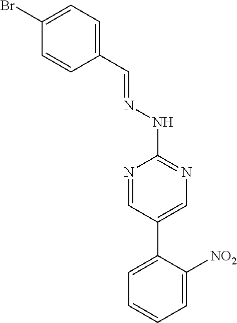 [SMILES: O=[N+]([O-])c1ccccc1-c1cnc(N/N=C/c2ccc(Br)cc2)nc1]

¹H NMR (500 MHz, CDCl₃) δ 9.07 (s, 1H), 8.60-8.55 (m, 2H), 8.30 (s, 1H), 7.85 (s, 1H), 7.80 (s, 1H), 7.61 (s, 1H), 7.60-7.55 (m, 2H), 7.53 (s, 1H), 7.40-7.34 (m, 2H); HRMS (ESI-TOF) calcd for C₁₇H₁₂FN₅O₂: [M+H] 337.0975 found 337.0968.

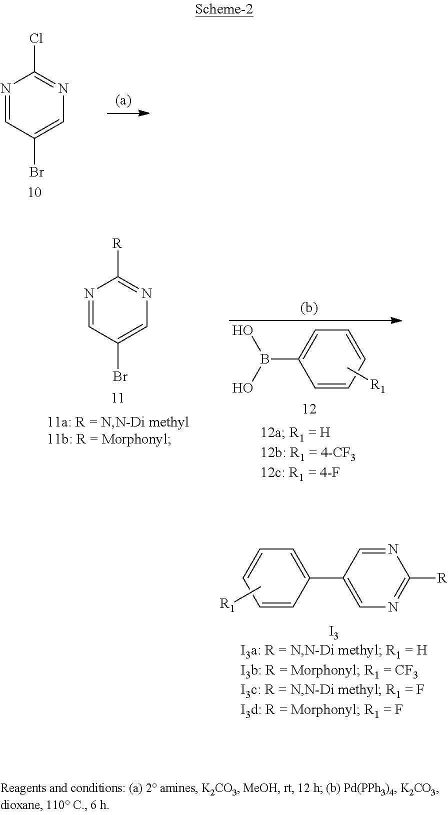

### General Procedure for the Synthesis of I₃a-d of Scheme 2:

To a solution of 11 (8.2 mmol) in dioxane (3 mL) under nitrogen atmosphere were added tetrakis(triphenylphosphine)palladium (0.047 g, 0.41 mmol), substituted phenylboronic acid 12 (0.98 mmol), and K₂CO₃ dissolved in 2 mL of water. The mixture was stirred under reflux for 6 h. The reaction mixture was diluted with EtOAc, filtered through celite, and washed with water. The combined organic layer was dried over anhydrous Na₂SO₄ and concentrated under reduced pressure. The crude products were purified on a silica gel column using hexane/EtOAc to furnish compound I₃a-d.

### N,N-Dimethyl-5-phenylpyrimidin-2-amine (I₃a, Scheme 2)

¹H NMR (400 MHz, CDCl₃) δ 8.49 (s, 2H), 7.38 (dt, J=15.2, 7.5 Hz, 4H), 7.26 (t, J=7.1 Hz, 1H), 3.17 (s, 6H); HRMS (ESI-TOF) calcd for C₂H₁₄N₃: [M+H] 200.1188 found 200.1187.

### 4-[5-(4-(Trifluoromethyl)phenyl)pyrimidin-2-yl]morpholine (I₃b, Scheme 2)

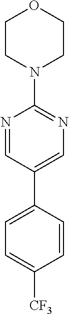 [SMILES: FC(F)(F)c1ccc(-c2cnc(N3CCOCC3)nc2)cc1]

¹H NMR (400 MHz, CDCl₃) δ 8.58 (s, 1H), 7.70 (d, J=8.2 Hz, 1H), 7.59 (d, J=8. Hz, 2H), 3.87 (m, 4H), 3.83-3.77 (m, 4H); HRMS (ESI-TOF) calcd for C₁₅H₁₅F₃N₃O: [M+H]⁺ 310.1167 found 310.1170.

### 5-(4-Fluorophenyl)-N,N-dimethylpyrimidin-2-amine (I₃c, Scheme 2)

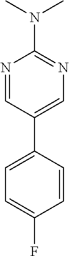 [SMILES: CN(C)c1ncc(-c2ccc(F)cc2)cn1]

¹H NMR (400 MHz, CDCl₃) δ 8.65 (s, 2H), 7.68-7.51 (m, 2H), 7.29-7.12 (m, 2H), 2.97 (s, 6H); HRMS (ESI-TOF) calcd for C₁₅H₁₂FN₃: [M+H]⁺ 217.1015 found 217.1036.

### 4-[5-(4-Fluorophenyl)pyrimidin-2-yl]morpholine (I₃d, Scheme 2)

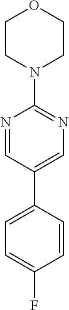 [SMILES: Fc1ccc(-c2cnc(N3CCOCC3)nc2)cc1]

¹H NMR (400 MHz, CDCl₃) 68.74 (m, 2H), 7.64-7.58 (m, 2H), 7.23-7.14 (m, 2H), 3.80-3.75 (m, 2H), 3.52-3.47 (m, 4H), 3.43-3.38 (m, 4H); HRMS (ESI-TOF) calcd for C₁₄H₁₄FN₃O: [M+H]⁺ 259.1121 found 259.1158.

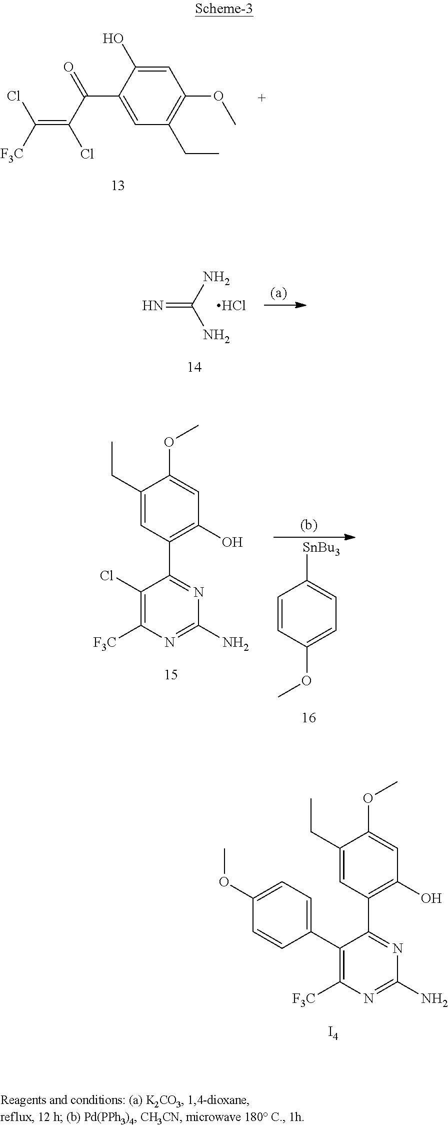

### General Procedure for the Synthesis of I₄ of Scheme 3

To a solution of 13 (8.2 mmol) and 14 in dioxane (3 mL) were refluxed for 12 h. The reaction mixture was diluted with EtOAc, filtered through celite, and washed with water. The combined organic layer was dried over anhydrous Na₂SO₄ and concentrated under reduced pressure. The crude product was further used without any purification. The compound 15 (8.2 mmol) was dissolved in acetonitrile (3 mL) under nitrogen atmosphere and then added tetrakis(triphenylphosphine)palladium (0.41 mmol), substituted compound 16 (0.98 mmol), and K₂CO₃ solution (dissolved in 2 mL of water). The mixture was heated 180° C. on microwave for 1 h. The reaction mixture was diluted with EtOAc, filtered through celite, and washed with water. The combined organic layer was dried over anhydrous Na₂SO₄ and concentrated under reduced pressure. The crude products were purified on a silica gel column using hexane/EtOAc to furnish compound I₄ as a white solid.

### 2-[2-amino-5-(4-methoxyphenyl)-6-(trifluoromethyl)pyrimidin-4-yl]-4-ethyl-5-methoxyphenol (I₄, Scheme 3)

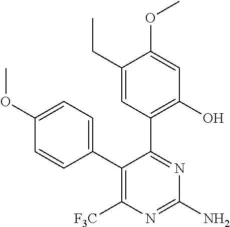 [SMILES: CCc1cc(-c2nc(N)nc(C(F)(F)F)c2-c2ccc(OC)cc2)c(O)cc1OC]

¹H NMR (500 MHz, CDCl₃) δ 7.61-7.55 (d, 2H), 7.31 (s, 1H), 7.07-7.04 (d, 2H), 6.61 (s, 1H), 6.55 (s, 1H), 3.86-3.79 (m, 6H), 2.61-2.52 (q, 2H), 1.68 (s, 2H), 1.31-1.25 (t, 3H); HRMS (ESI-TOF) calcd for C₂₁H₂₀F₃N₃O₃: [M+H] 419.1457 found 419. 1479.

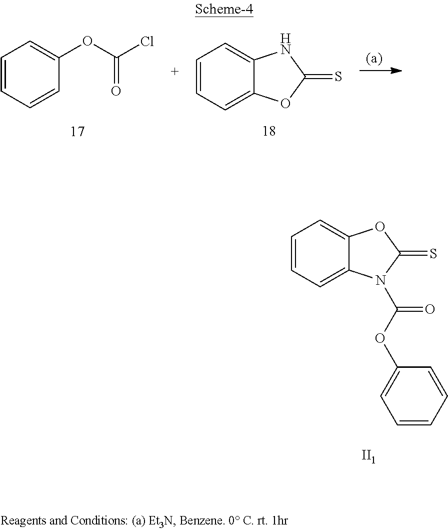 [SMILES: O=C(Cl)Oc1ccccc1 ; S=c1[nH]c2ccccc2o1 ; O=C(Oc1ccccc1)n1c(=S)oc2ccccc21 ; "II1"]

### General Procedure for the Synthesis of II) of Scheme 4:

To a solution of benzo[d]oxazole-2(3H)-thione 18 (2.6 mmol) in ethylacetate was added phenyl chloroformate 17 slowly at 0° C. (4.00 mmol) and catalytic amounts of pyridine. The resulting mixture was stirred at rt for 1 h. After completion of the reaction (reaction monitored by TLC) the solvent was removed under vacuum, and the compound was extracted with EtOAc. The combined organic layer was dried over anhydrous Na₂SO₄ and concentrated under reduced pressure. The crude products were purified on a silica gel column using hexane/EtOAc to furnish compound II).

### Phenyl 2-thioxobenzo[d]oxazole-3(2H)-carboxylate (II₁), Scheme 4)

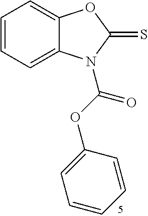 [SMILES: O=C(Oc1ccccc1)n1c(=S)oc2ccccc21]

¹H NMR (400 MHz, CDCl₃) δ 7.82-7.76 (m, 1H), 7.45-7.37 (m, 2H), 7.33-7.25 (m, 6H). LC-MS (ESI+): m\z 272.0303 [M+H]⁺.

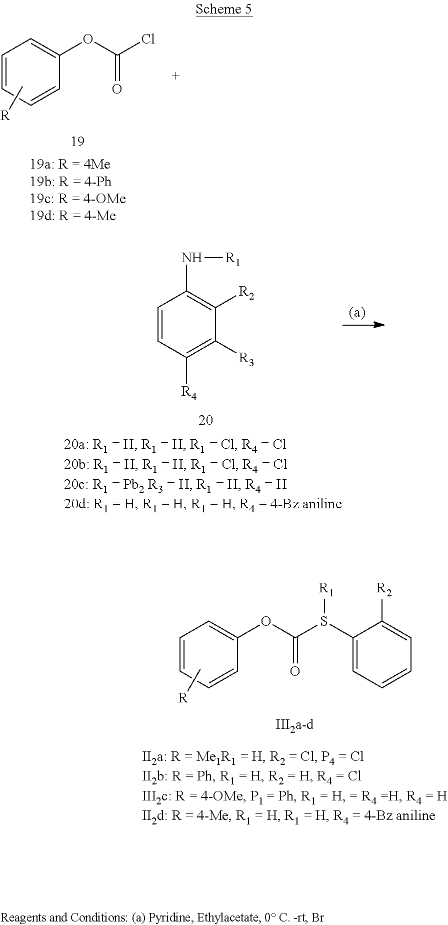

### General Procedure for the Synthesis of II₂a-d of Scheme 5

To a solution of substituted anilines 20 (2.6 mmol) in ethylacetate was added substituted phenyl chloroformate 19 slowly at 0° C. (4.00 mmol) and catalytic amounts of pyridine. The resulting mixture was stirred at rt for 1 h. After completion of the reaction (reaction monitored by TLC) the solvent was removed under vacuum, and the compound was extracted with EtOAc. The combined organic layer was dried over anhydrous Na₂SO₄ and concentrated under reduced pressure. The crude products were purified on a silica gel column using hexane/EtOAc to furnish compound II₂a-d.

### p-Tolyl (3,4-dichlorophenyl)carbamate (II₂a, Scheme 5)

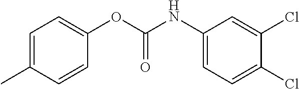 [SMILES: Cc1ccc(OC(=O)Nc2ccc(Cl)c(Cl)c2)cc1]

¹H NMR (400 MHz, CDCl₃) δ 10.11(s, 1H), 7.60 (s, 1H), 7.35 (d, J=2.2 Hz, 2H), 7.15-7.03 (m, 2H), 7.03-6.90 (m, 2H), 2.41-2.18 (m, 3H). LC-MS (ESI+): m\z 296.0240 [M+H]⁺.

### [1,1′-biphenyl]-4-yl(3,4-dichlorophenyl)carbamate (II₂b, Scheme 5)

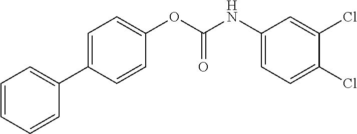 [SMILES: O=C(Nc1ccc(Cl)c(Cl)c1)Oc1ccc(-c2ccccc2)cc1]

¹H NMR (400 MHz, CDCl₃) 610.11 (s, 1H), 7.83 (s, 1H), 7.76-7.47 (m, 4H), 7.47-7.41 (m, 2H), 7.35 (d, J=3.4 Hz, 2H), 7.26-7.04 (m, 3H). LC-MS (ESI+): m\z 358.0396 [M+H]⁺.

### 4-Methoxyphenyl diphenylcarbamate (II₂c, Scheme 5)

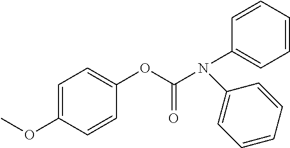 [SMILES: COc1ccc(OC(=O)N(c2ccccc2)c2ccccc2)cc1]

¹H NMR (400 MHz, CDCl₃) δ 7.33-7.23 (m, 1H), 7.15-7.07 (m, 1H), 7.03-6.95 (m, 1H), 6.88-6.81 (m, 1H), 6.81-6.75 (m, 1H), 6.75-6.67 (m, 1H), 3.74 (s, 1H). LC-MS (ESI+): m\z 320.1281 [M+H]⁺.

### Di-p-tolyl [methylenebis(4,1-phenylene)]dicarbamate (II₂d, Scheme 5)

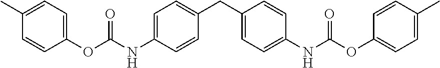

¹H NMR (400 MHz, CDCl₃) δ 9.86 (s, 2H), 7.52-7.38 (m, 4H), 7.38-7.29 (m, 4H), 7.19-7.07 (m, 4H), 7.04-6.95 (m, 4H), 3.91-3.57 (s, 2H), 2.50-2.17 (s, 6H). LC-MS (ESI+): m\z 467.1965 [M+H]⁺.

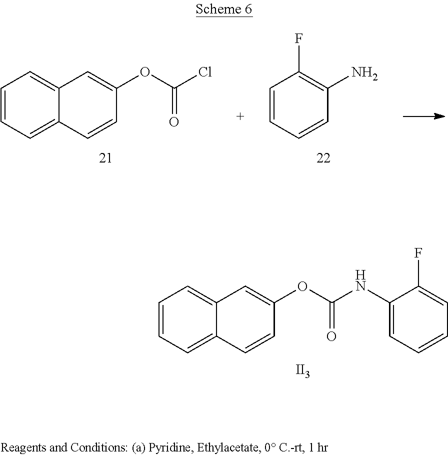

### General Procedure for the Synthesis of II₃ of Scheme 6:

To a solution of substituted aniline 22 (2.6 mmol) in ethylacetate was added substituted phenyl chloroformate 21 slowly at 0° C. (4.00 mmol) and catalytic amounts of pyridine. The resulting mixture was stirred at rt for 1 h. After completion of the reaction (reaction monitored by TLC) the solvent was removed under vacuum, and the compound was extracted with EtOAc. The combined organic layer was dried over anhydrous Na₂SO₄ and concentrated under reduced pressure. The crude products were purified on a silica gel column using hexane/EtOAc to furnish compound II₃.

### Naphthalen-2-yl (2-fluorophenyl)carbamate (II₃, Scheme 6)

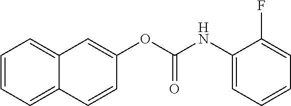 [SMILES: O=C(Nc1ccccc1F)Oc1ccc2ccccc2c1]

¹H NMR (400 MHz, CDCl₃) δ 9.86 (s, 1H), 7.78-7.68 (m, 1H), 7.52 (s, 1H), 7.34 (t, J=5.1 Hz, 1H), 7.10 (dd, J=15.8, 3.3 Hz, 1H). LC-MS (ESI+): m\z 282.0295 [M+H]⁺.

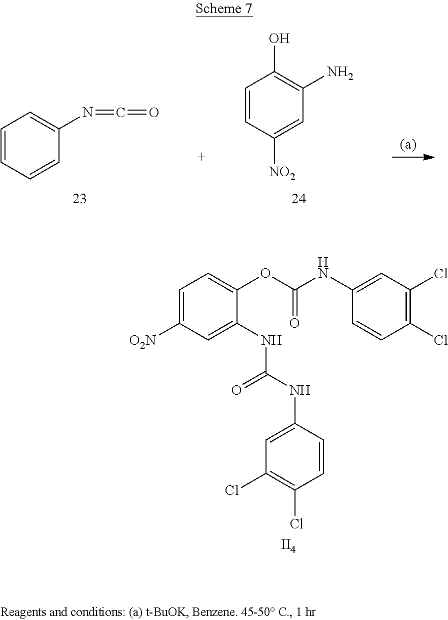

### General Procedure for the Synthesis of II₄ of Scheme 7:

To a solution of phenylisocyante 23(4.2 mmol) in benzene was added potassium tert-butoxide (10 mol %) slowly and then 2-amino-4-nitrophenol 24 (4.2 mmol) was added. The resulting mixture was stirred at 45-50° C. for 1 h. After completion of the reaction (reaction monitored by TLC) the solvent was removed under vacuum, and the compound was extracted with EtOAc. The combined organic layer was dried over anhydrous Na₂SO₄ and concentrated under reduced pressure. The crude products were purified on a silica gel column using hexane/EtOAc to furnish compound II₄.

### 2-[3-(3,4-dichlorophenyl)ureido]-4-nitrophenyl (3,4-dichlorophenyl)carbamate (II₄, Scheme 7)

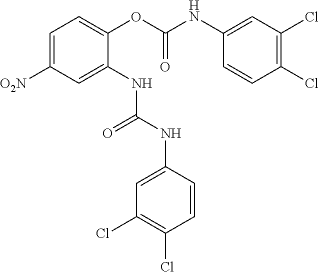 [SMILES: O=C(Nc1ccc(Cl)c(Cl)c1)Nc1cc([N+](=O)[O-])ccc1OC(=O)Nc1ccc(Cl)c(Cl)c1]

¹H NMR (400 MHz, DMSO-d₆) δ 9.23 (s, 1H), 8.59 (s, 1H), 8.41 (s, 1H), 8.05 (s, 1H), 7.81 (s, 1H), 7.55 (d, J=34.9 Hz, 2H), 7.46-7.32 (m, 3H), 7.28 (s, 1H), 7.01 (s, 1H). LC-MS (ESI+): m\z 528.9635 [M+H]⁺.

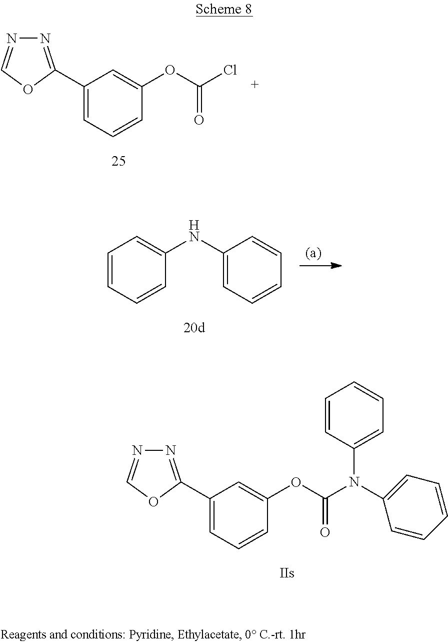 [SMILES: O=C(Cl)Oc1cccc(-c2nnco2)c1 ; c1ccc(Nc2ccccc2)cc1 ; O=C(Oc1cccc(-c2nnco2)c1)N(c1ccccc1)c1ccccc1]

### General Procedure for the Synthesis of II₅ of Scheme 8

To a solution of substituted aniline 20d (2.6 mmol) in ethylacetate was added substituted phenyl chloroformate 25 slowly at 0° C. (4.00 mmol)and catalytic amounts of pyridine. The resulting mixture was stirred at rt for 1 h. After completion of the reaction (reaction monitored by TLC) the solvent was removed under vacuum, and the compound was extracted with EtOAc. The combined organic layer was dried over anhydrous Na₂SO₄ and concentrated under reduced pressure. The crude products were purified on a silica gel column using hexane/EtOAc to furnish compound II₅.

### 3-[(1,3,4-oxadiazol-2-yl)phenyl]diphenylcarbamate (II₅, Scheme 8)

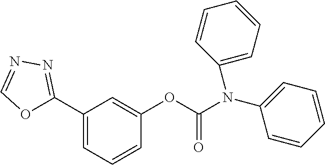 [SMILES: O=C(Oc1cccc(-c2nnco2)c1)N(c1ccccc1)c1ccccc1]

¹H NMR (400 MHz, DMSO) δ 9.45 (s, 1H), 7.82 (s, 1H), 7.51 (s, 1H), 7.42-7.26 (m, 10H), 7.18 (m, 1H), 7.14-7.03 (m, 2H).). LC-MS (ESI+): m\z 358.1186 [M+H]⁺.

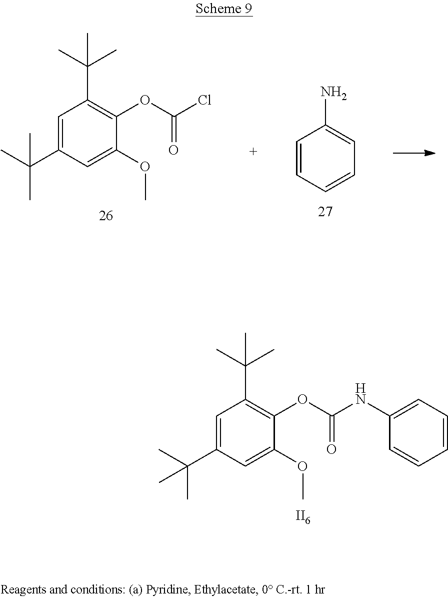

### General Procedure for the Synthesis of II₆ of Scheme 9:

To a solution of substituted aniline 27 (2.6 mmol) in ethyl acetate was added substituted phenyl chloroformate 26 slowly at 0° C. (4.00 mmol) and catalytic amounts of pyridine. The resulting mixture was stirred at rt for 1 h. After completion of the reaction (reaction monitored by TLC) the solvent was removed under vacuum, and the compound was extracted with EtOAc. The combined organic layer was dried over anhydrous Na₂SO₄ and concentrated under reduced pressure. The crude products were purified on a silica gel column using hexane/EtOAc to furnish compound II₆.

### 2,4-di-tert-butyl-6-methoxyphenyl phenylcarbamate (II₆, Scheme 9)

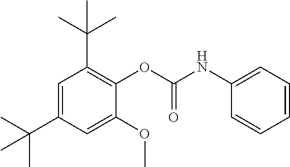 [SMILES: COc1cc(C(C)(C)C)cc(C(C)(C)C)c1OC(=O)Nc1ccccc1]

¹H NMR (400 MHz, CDCl₃) δ 9.86(s, 1H), 7.60-7.34 (m, 4H), 7.16 (m, 1H), 7.03 (s, 1H), 6.79 (s, 1H), 3.81-3.74 (m, 3H), 1.43-1.36 (m, 9H), 1.35-1.29 (m, 9H); LC-MS (ESI+): m\z 356.2220 [M+H]⁺.

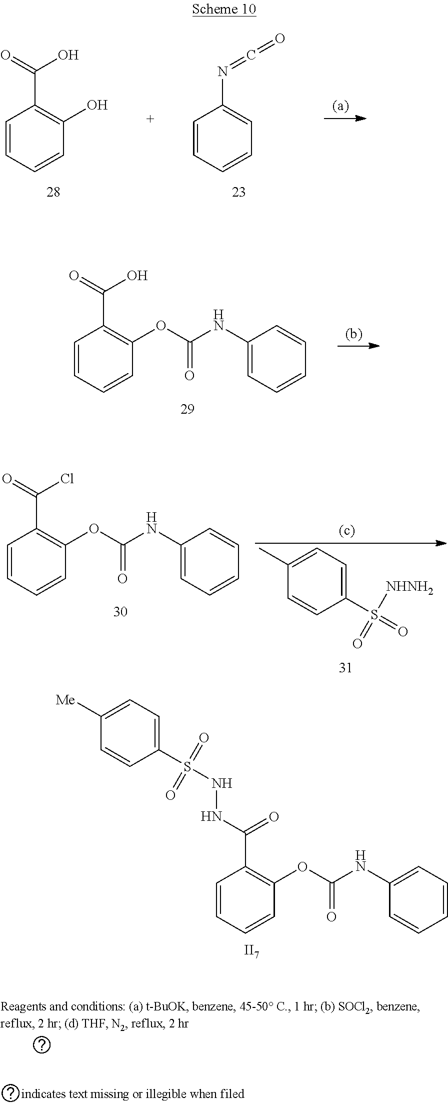

### General Procedure for the Synthesis of II₇ of Scheme 10):

To a solution of acid 29 (2 mmol) in benzene (5 mL) was added thionyl chloride (6 mmol). The reaction mixture was stirred under reflux for 2 hrs and concentrated to give the crude acid chlorides 30 as yellow liquids. This liquid was dissolved in dry THF (5 ml) added drop wise to p-tosylhydrazine 31 (1 mmol). After being refluxed for 2-4 hrs the reaction mixture was concentrated. Reaction completion monitored by TLC. The resulting mixture was partition between ethyl acetate and water and the organic layer was separated and finally washed with brine, dried over anhydrous sodium sulphate, and filtered. The filtrate was concentrated under reduced pressure. The residue was purified by silica gel column chromatography by using gradient mixture of ethyl acetate and hexane to afford compounds 117.

### 2-[(2-Tosylhydrazine-1-carbonyl)phenyl]phenylcarbamate (II₇, Scheme 10)

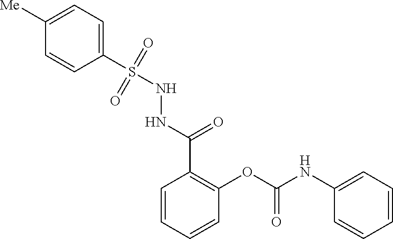 [SMILES: Cc1ccc(S(=O)(=O)NNC(=O)c2ccccc2OC(=O)Nc2ccccc2)cc1]

¹H NMR (400 MHz, DMSO) δ 8.99 (s, 1H), 8.12-7.99 (m, 1H), 7.85 (s, 2H), 7.69-7.46 (m, 4H), 7.46-7.33 (m, 5H), 7.30 (s, 1H), 7.11 (s, 1H), 4.56 (s, 1H), 2.41-2.35 (m, 3H); LC-MS (ESI+): m\z 426.1118 [M+H]⁺.

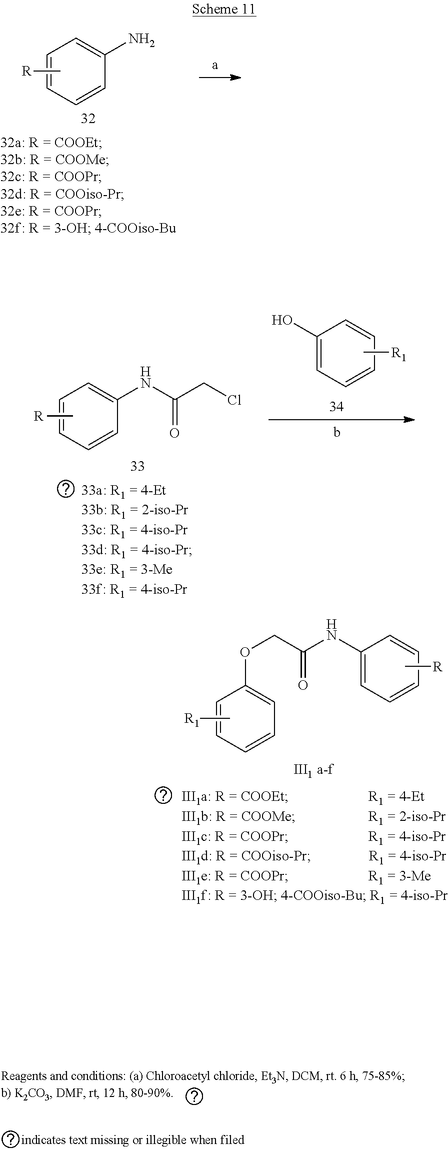

### General Procedure for the Synthesis of III₁a-g of Scheme 11:

To a solution of substituted anilines 32 (5 mmol) in DCM (5 ml) and triethylamine (10 mmol) was added chloroacetyl chloride (7.5 mmol). The reaction was stirred at room temperature for 6 hours then diluted with water and extract with DCM, the organic layer was separated and washed with brine, dried over anhydrous sodium sulphate and filtered. The filtrate was concentrated to give the crude 2-chloro amides 33 as a yellow liquid. This yellow liquid was dissolved in dimethyl formamide (5 ml) was added K₂CO₃ (15 mmol) and corresponding substituted phenols 34 at room temperature. After being stirred at room temperature for 12 h, the reaction mixture was diluted with ice water and extract with ethyl acetate, the organic layer was separated and washed with brine, dried over anhydrous sodium sulphate and filtered. The filtrate was concentrated under reduced pressure. The residue was purified by silica gel column chromatography using EtOAc/Hexane as eluents to get compounds III₁a-f.

### Ethyl 4-[2-(4-ethylphenoxy)acetamido]benzoate (III₁a, Scheme 11)

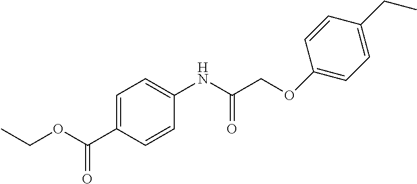 [SMILES: CCOC(=O)c1ccc(NC(=O)COc2ccc(CC)cc2)cc1]

¹H NMR (400 MHz, CDCl₃) δ 8.45 (s, 1H), 8.04 (d, J=8.7 Hz, 2H), 7.68 (d, J=8.7 Hz, 2H), 7.18 (d, J=8.5 Hz, 2H), 6.92 (d, J=8.6 Hz, 2H), 4.60 (s, 2H), 4.37 (q, J=7.1 Hz, 2H), 2.62 (q, J=7.6 Hz, 2H), 1.39 (t, J=7.1 Hz, 3H), 1.22 (t, J=7.6 Hz, 3H);HRMS (ESI-TOF) calcd for C₁₉H₂₁NO₄ [M+H]⁺ 328.1549, found 328.1567.

### Methyl 4-[2-(2-isopropylphenoxy)acetamido]benzoate (III₁b, Scheme 11)

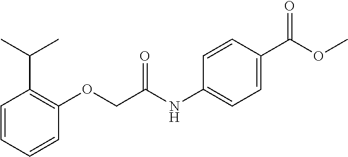 [SMILES: COC(=O)c1ccc(NC(=O)COc2ccccc2C(C)C)cc1]

¹H NMR (400 MHz, CDCl₃) δ 8.49 (s, 1H), 8.03 (d, J=8.65 Hz, 2H), 7.64 (d, J=8.65 Hz, 2H), 7.24 (d, J=7.4 Hz, 1H), 7.19 (t, J=8.02 Hz, 1H), 7.06 (t, J=7.38 Hz, 1H), 6.84(d, J=8.03 Hz, 1H), 4.64 (s, 2H), 3.91 (s, 3H), 3.34-3.28 (m, 1H), 1.32 (d, J=7.38 Hz, 6H); HRMS (ESI-TOF) calcd for C₁₉H₂₁NO₄ [M+H]⁺ 328.1549, found 328.1565.

### Propyl 4-[2-(4-isopropylphenoxy)acetamido]benzoate (III₁c, Scheme 11)

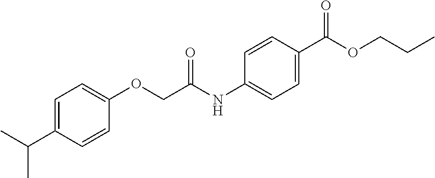 [SMILES: CCCOC(=O)c1ccc(NC(=O)COc2ccc(C(C)C)cc2)cc1]

¹H NMR (400 MHz, CDCl₃) δ 8.47 (s, 1H), 8.07 (d, J=8.7 Hz, 2H), 7.71 (d, J=8.7 Hz, 2H), 7.23 (d, J=8.6 Hz, 2H), 6.95 (d, J=8.6 Hz, 2H), 4.63 (s, 2H), 4.30 (t, J=6.7 Hz, 2H), 2.95-2.88 (m, 1H), 1.88-1.76(m, 2H), 1.26 (d, J=6.9 Hz, 6H), 1.05 (t, J=7.4 Hz, 3H); HRMS (ESI-TOF) calcd for C₂₁H₂₅NO₄ [M+Na]⁺ 378.1682, found 378.1681.

### Isopropyl 4-[2-(4-isopropylphenoxy)acetamido]benzoate (III₁d, Scheme 11)

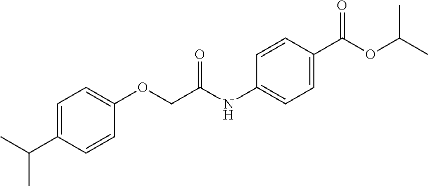 [SMILES: CC(C)OC(=O)c1ccc(NC(=O)COc2ccc(C(C)C)cc2)cc1]

¹H NMR (400 MHz, CDCl₃) δ 8.47 (s, 1H), 8.06 (d, J=8.7 Hz, 2H), 7.71 (d, J=8.7 Hz, 2H), 7.23 (d, J=8.6 Hz, 2H), 6.95 (d, J=8.7 Hz, 2H), 5.30-5.23(m, 1H), 4.63 (s, 2H), 2.95-2.88 (m, 1H), 1.39 (d, J=6.3 Hz, 6H), 1.26 (d, J=6.9 Hz, 6H); HRMS (ESI-TOF) calcd for C₂₁H₂₅NO₄ [M+Na]⁺ 378.1682, found 378.1645.

### Propyl 4-[2-(m-tolyloxy)acetamido]benzoate (III₁e, Scheme 11)

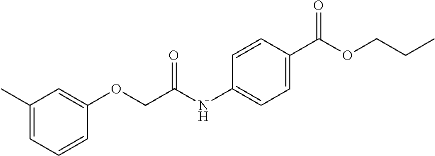 [SMILES: CCCOC(=O)c1ccc(NC(=O)COc2cccc(C)c2)cc1]

¹H NMR (400 MHz, CDCl₃) δ 8.44 (s, 1H), 8.05 (d, J=8.7 Hz, 2H), 7.69 (d, J=8.7 Hz, 2H), 7.23 (t, J=7.8 Hz, 1H), 6.89 (d, J=7.5 Hz, 1H), 6.84-6.77 (m, 2H), 4.62 (s, 2H), 4.27 (t, J=6.7 Hz, 2H), 2.37 (s, 3H), 1.85-1.74 (m, 2H), 1.03 (t, J=7.4 Hz, 3H); HRMS (ESI-TOF) calcd for C₁₉H₂₁NO₄ [M+H]⁺ 328.1549, found 328.1541.

### Isobutyl 2-hydroxy-4-[2-(4-isopropylphenoxy)acetamido]benzoate (III₁f, Scheme 11):

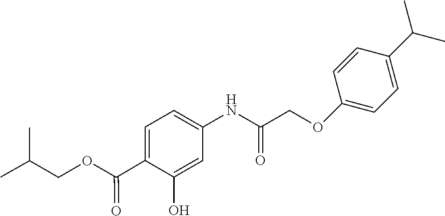 [SMILES: CC(C)COC(=O)c1ccc(NC(=O)COc2ccc(C(C)C)cc2)cc1O]

¹H NMR (400 MHz, CDCl₃) δ 7.85 (s, 4H), 7.36-7.15 (m, 8H), 7.12 (s, 4H), 7.04 (s, 4H), 7.01-6.90 (m, 12H), 4.93-4.87 (m, 8H), 4.37 (s, 4H), 4.28-4.03 (m, 8H), 3.03 (s, 3H), 2.27 (s, 3H), 1.42-1.20 (m, 24H), 1.13-0.90 (m, 24H). LC-MS (ESI+): m\z 386.1962 [M+H]⁺.

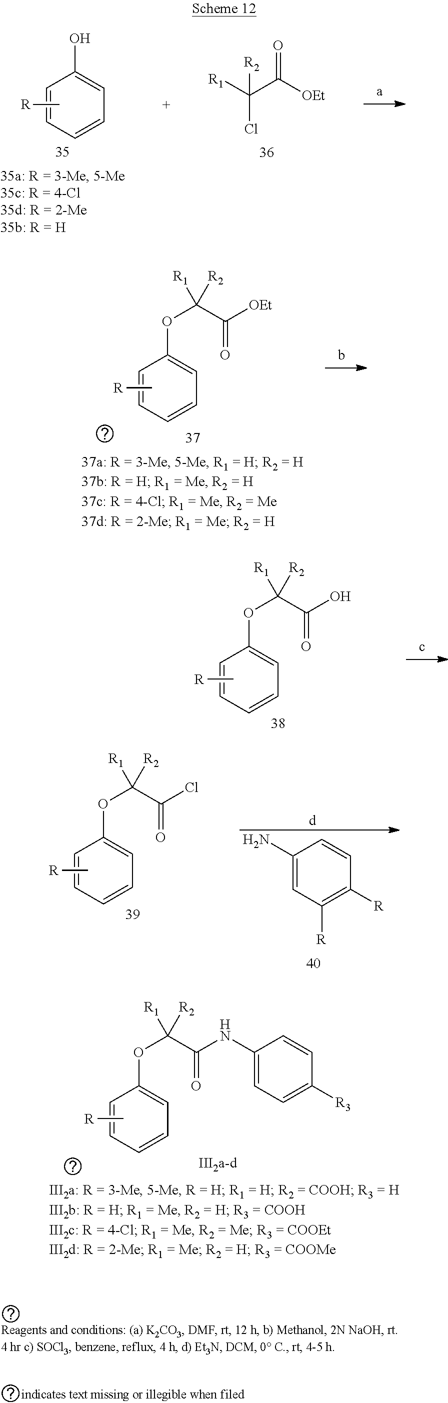

### General Procedure for the Synthesis of III₂a-d, Scheme 12):

To a suspension of substituted phenoxyalkyl acids 38 (2 mmol) in benzene (5 mL) was added thionyl chloride (6 mmol). The reaction mixture was stirred under reflux for 5 h and concentrated to give the crude acid chlorides 39 as yellow liquids. This liquid was dissolved in dry DCM (5 ml) added drop wise to the substituted anilines 40 (1 mmol) in triethylamine (3 mmol) and DCM (5 ml) at 0° C. After being stirred at room temperature for 4 h the reaction mixture was concentrated. The resulting mixture was partition between ethyl acetate and water and the organic layer was separated. The organic layer washed with saturated bicarbonate solution and washed with 2N HCl and finally washed with brine, dried over anhydrous sodium sulphate, and filtered. The filtrate was concentrated under reduced pressure. The residue was purified by silica gel column chromatography by using gradient mixture of ethyl acetate and hexane to afford compounds III₂a-d.

### 4-[2-(3,5-dimethylphenoxy)acetamido]-2-hydroxybenzoic acid (III₂a, Scheme 12)

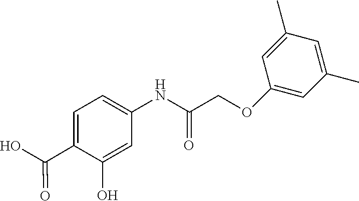 [SMILES: Cc1cc(C)cc(OCC(=O)Nc2ccc(C(=O)O)c(O)c2)c1]

¹H NMR (400 MHz, DMSO) δ 9.88 (s, 1H), 8.05 (s, 1H), 7.49 (s, 1H), 7.10 (s, 1H), 6.78-6.68 (m, 3H), 4.84-4.78 (m, 2H), 2.38-2.32 (m, 6H). LC-MS (ESI+): m\z 316.1179 [M+H]⁺.

### 4-(2-Phenoxypropanamido)benzoic acid (III₂b, Scheme 12)

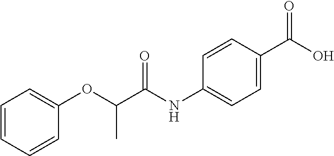 [SMILES: CC(Oc1ccccc1)C(=O)Nc1ccc(C(=O)O)cc1]

¹H NMR (400 MHz, Acetone-d₆) δ 9.72 (s, 1H), 7.98 (d, J=8.7 Hz, 2H), 7.86 (d, J=8.7 Hz, 2H), 7.36-7.27 (m, 2H), 7.03 (d, J=7.9 Hz, 2H), 6.99 (t, J=7.4 Hz, 1H), 4.92 (q, J=6.7 Hz, 1H), 1.63 (d, J=6.7 Hz, 3H);HRMS (ESI-TOF) calcd for C₁₆H₁₅NO₄ [M+H]⁺ 286.1079, found 286.1074.

### Ethyl 4-[2-(4-chlorophenoxy)-2-methylpropanamido]benzoate (III₂c, Scheme 12)

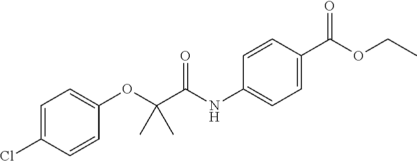 [SMILES: CCOC(=O)c1ccc(NC(=O)C(C)(C)Oc2ccc(Cl)cc2)cc1]

¹H NMR (400 MHz, CDCl₃) δ 8.73 (s, 1H), 8.05 (d, J=8.4 Hz, 2H), 7.69 (d, J=8.6 Hz, 2H), 7.29 (d, J=8.4 Hz, 2H), 6.95 (d, J=8.6 Hz, 2H), 4.38 (q, J=7.1 Hz, 2H), 1.59 (s, 6H), 1.41 (t, J=7.1 Hz, 3H); HRMS (ESI-TOF) calcd for C₁₈H₁₈ClNO₄ [M+H]⁺ 348.1002, found 348.0999.

### Methyl 4-[2-(o-tolyloxy)propanamido]benzoate (III₂d, Scheme 12)

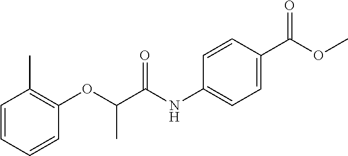 [SMILES: COC(=O)c1ccc(NC(=O)C(C)Oc2ccccc2C)cc1]

¹H NMR (400 MHz, CDCl₃) δ 8.52 (s, 1H), 8.04 (d, J=8.7 Hz, 2H), 7.67 (d, J=8.7 Hz, 2H), 7.27-7.16 (m, 2H), 6.99 (t, J=7.4 Hz, 1H), 6.87 (d, J=8.2 Hz, 1H), 4.82 (q, J=6.7 Hz, 1H), 3.92 (s, 3H), 2.38 (s, 3H), 1.69 (d, J=6.8 Hz, 3H); HRMS (ESI-TOF) calcd for C₁₈H₁₉NO₄ [M+H]⁺ 314.1392, found 314.1428.

### Synthesis of the Intermediates

### General Procedure for the Synthesis of Intermediate 3 of I₁a-f (Scheme 1):

To a solution of compound 1 (1.93 mg, 10 mmol) in dry benzene at 0° C. was added ethane thiol 2 (0.93 g, 1.5 mmol), followed by NaH (0.36 mg, 1.5 mmol) in portion wise. The resulting mixture was stirred at room temperature for 3 h. After completion of the reaction (reaction monitored by TLC) the solvent was removed under vacuum, it was quenched with ice-cold water and extracted with ethylacetate. The combined organic layer was dried over anhydrous Na₂SO₄ and concentrated under reduced pressure. The crude products were purified on a silica gel column using hexane/EtOAc to give compound 3 as a white liquid (1.9 g, 88%). ¹H NMR (500 MHz, CDCl₃) δ 8.90-8.73 (s, 2H), 3.16-3.00 (q, 2H), 1.41-1.23 (t, 3H); Mass (ESI+) 217.9513 [M+H].

### General Procedure for the Synthesis of Intermediate 5a-f of I₁a-f (Scheme 1)

To a solution of 3 (8.7 mmol) in dioxane (4 mL) under nitrogen atmosphere were added tetrakis(triphenylphosphine)palladium (0.44 mmol), substituted phenylboronic acid 4 (10.5 mmol), and K₂CO₃ (8 mmol), dissolved in 4 mL of water. The mixture was stirred under reflux for 6 h. The reaction mixture was diluted with EtOAc, filtered through celite, and washed with water. The combined organic layer was dried over anhydrous Na₂SO₄ and concentrated under reduced pressure. The crude products were purified on a silica gel column using hexane/EtOAc to furnish compound 5.

### 2-(Ethylthio)-5-(4-nitrophenyl)pyrimidine 5a of I₁ (Scheme 1)

¹H NMR (500 MHz, CDCl₃) δ 8.87-8.58 (s, 2H), 8.46-8.20 (d, 2H), 7.93-7.64 (d, 2H), 3.18-3.05 (q, 2H), 1.38-1.29 (t, 3H); Mass (ESI+) 261.05 [M+H].

### 2-(Ethylthio)-5-(4-trifluoromethylphenyl)pyrimidine 5b of I₁ (Scheme 1)

¹H NMR (500 MHz, CDCl₃) δ 8.51-8.45 (s, 2H), 8.21-8.13 (d, 2H), 7.73-7.54 (d, 2H), 3.03-2.96 (q, 2H), 1.31-1.22 (t, 3H); Mass (ESI+) 255.09 [M+H].

### 2-(Ethylthio)-5-(4-trifluoromethoxyphenyl)pyrimidine 5c of I₁ (Scheme 1)

¹H NMR (500 MHz, CDCl₃) δ 8.57-8.49 (s, 2H), 8.21-8.17 (d, 2H), 7.29-7.13 (d, 2H), 3.03-2.96 (q, 2H), 1.31-1.22 (t, 3H); Mass (ESI+) 271.16 [M+H].

### 2-(Ethylthio)-5-(4-methoxyphenyl)pyrimidine 5d of I₁ (Scheme 1)

¹H NMR (500 MHz, CDCl₃) δ 8.51-8.45 (s, 2H), 8.19-8.14 (d, 2H), 7.09-6.94 (d, 2H), 3.21 (s, 3H), 3.03-2.96 (q, 2H), 1.31-1.22 (t, 3H); Mass (ESI+) 217.12 [M+H].

### 2-(Ethylthio)-5-(3-nitrophenyl)pyrimidine 5e of I₁ (Scheme 1)

¹H NMR (500 MHz, CDCl₃) δ 8.87-8.58 (s, 2H), 8.48-8.23 (m, 4H), 3.18-3.05 (q, 2H), 1.38-1.29 (t, 3H); Mass (ESI+) 261.05 [M+H].

### 2-(Ethylthio)-5-(2-nitrophenyl)pyrimidine 5f of I₁ (Scheme 1)

¹H NMR (500 MHz, CDCl₃) δ 8.87-8.58 (s, 2H), 8.46-8.10 (m, 4H), 3.18-3.05 (q, 2H), 1.38-1.29 (t, 3H); Mass (ESI+) 261.05 [M+H].

### General Procedure for the Synthesis of Intermediate 6a-f of I₁ (Scheme 1)

To a solution of compound 5 (7.6 mmol) in DCM at 0° C. was added 3-chloroperbenzoic acid (15.3 mmol) in portion wise. The resulting mixture was stirred at room temperature for 3 h. After completion of the reaction (reaction monitored by TLC) the solvent was removed under vacuum, saturated bicarbonate solution (10 mL) was added, and the reaction mixture was extracted with EtOAc. The combined organic layer was dried over anhydrous Na₂SO₄ and concentrated under reduced pressure to give compound 6. The crude products were further used without any purification.

General procedure for the synthesis of intermediate 7a-f of I₁ (Scheme 1)

To a solution of compound 6 (5.1 mmol) in THF was added hydrazine hydrate (0.5 g, 10.2 mmol). The resulting mixture was stirred at 60° C.-70° C. for 3 h. After completion of the reaction (reaction monitored by TLC) the solvent was removed under vacuum, and the reaction mixture was extracted with EtOAc. The combined organic layer was dried over anhydrous Na₂SO₄ and concentrated under reduced pressure to give compound 7. The crude products were further used without any purification.

### General Procedure for the Synthesis of Intermediate 11a-b of I₃a-d (Scheme 2)

To a solution of compound 10 (1 mmol) in methanol was added 2° amine (1.5 mmol), and K₂CO₃ (1 mmol). The resulting mixture was stirred at room temperature for 1 h. After completion of the reaction (reaction monitored by TLC) the solvent was removed under vacuum, and the compound was extracted with EtOAc. The combined organic layer was dried over anhydrous Na₂SO₄ and concentrated under reduced pressure. The crude products were purified on a silica gel column using hexane/EtOAc to get compound 11.

### General Procedure for the Synthesis of Intermediate 29 of II₇ (Scheme 10):

To a solution of phenylisocyante 23 (4.2 mmol) in benzene was added potassium tert-butoxide (10 mol %) slowly and then salicylic acid 28 (4.2 mmol) was added. The resulting mixture was stirred at 45-50° C. for 1 h. After completion of the reaction (reaction monitored by TLC) the solvent was removed under vacuum, and the compound was extracted with EtOAc. The combined organic layer was dried over anhydrous Na₂SO₄ and concentrated under reduced pressure. The crude products were purified on a silica gel column using hexane/EtOAc to afford compound 29.

### General Procedure for the Synthesis of Intermediate 38a-d of III₂a-d (Scheme 12):

To a solution of substituted phenols 35 (10 mmol) in DMF (10 ml) was added K₂CO₃ (20 mmol) and substituted 2-chloroethylacetate 36 (12 mmol) at room temperature. The reaction mixture was stirred at room temperature for 12 h, and then the reaction mixture was poured in ice water, extracted with ethyl acetate twice. Combined the organic layers washed with brine solution and dried over anhydrous Na₂SO₄, and concentrated to get the crude compounds 37. These crude compounds 37 were dissolved in methanol (10 ml) was added 2N NaOH (6 ml) at room temperature and stirred for 12 h. Solvent was evaporated under vacuum and the resulting mixture was acidified with 2N HCl and then solids were filtered off. The solids were washed with water and hexane and dried for some time under vacuum to get the pure substituted 2-phenoxyacetic acids 38a-d in 80-90%.

### 2-(3,5-Dimethylphenoxy)acetic acid (38a, Scheme 12)

¹H NMR (400 MHz, CD₃OD) δ 7.01 (s, 1H), 6.77-6.67 (m, 2H), 4.94-4.87 (s, 2H), 2.39-2.32 (m, 6H); LC-MS (ESI+): m\z 386.1962 [M+H]⁺.

### 2-Phenoxypropanoic acid (38b, Scheme 12)

¹H NMR (400 MHz, CD₃OD) δ 7.34 (t, J=8.0 Hz, 2H), 7.01 (t, J=7.4 Hz, 1H), 6.97 (d, J=7.9 Hz, 2H), 4.75 (q, J=6.75 Hz, 1H), 1.64 (d, J=6.75 Hz, 3H); LC-MS (ESI+): m\z 167.08 [M+H]⁺.

### 2-(4-Chlorophenoxy)-2-methylpropanoic acid (38c, Scheme 12)

¹H NMR (400 MHz, CD₃OD) δ 7.29 (d, J=8.2 Hz, 2H), 6.95 (d, J=8.2 Hz, 2H), 1.59 (s, 6H);LC-MS (ESI+): m\z 215.12 [M+H]⁺.

### 2-(o-Tolyloxy)propanoic acid (38d, Scheme 12)

¹H NMR (400 MHz, CD₃OD) δ 7.28-7.15 (m, 2H), 6.99 (t, J=7.4 Hz, 1H), 6.87 (d, J=8.2 Hz, 1H), 4.82 (q, J=6.7 Hz, 1H), 1.69 (d, J=6.8 Hz, 3H); LC-MS (ESI+): m\z 181.09 [M+H]⁺.

### EXAMPLES

The following examples provide the details about the, applications of the compounds of the present disclosure. It should be understood the following is representative only, and that the invention is not limited by the details set forth in these examples.

### Example 1

### Biological Activity/Assay

Through an in vitro biochemistry screen, small molecules were identified which can target Nef interaction with CD80 and CD86. Out of the 1061 compounds, three scaffolds were identified. Compounds from these scaffolds (Formula I, Formula II, and Formula III) were validated in (a.) Primary biochemistry screen; and (b.) Cell-based screen.

### Validation of Compounds in Primary Screen

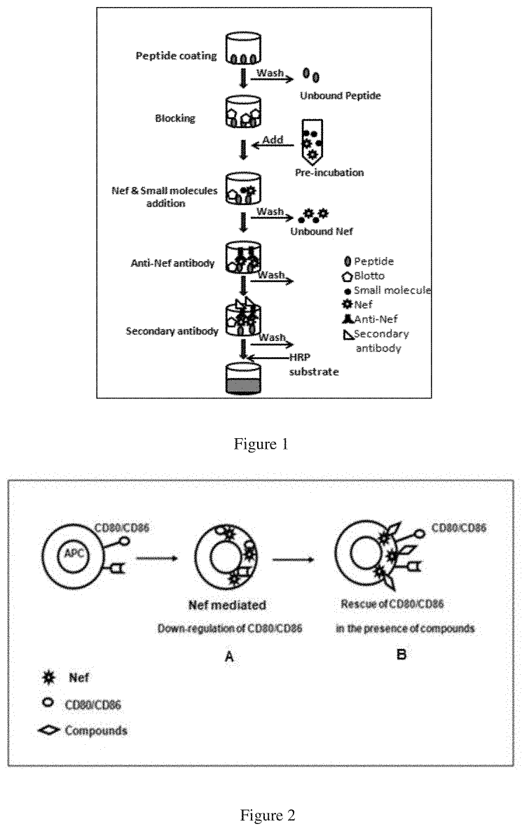

Standard ELISA assay was used to validate the compounds as a primary screen. The basic steps of the assay are outlined in FIG. 1. The cytosolic peptides CD80, CD86 and CD74 are coated on a microplate, while CD80 and CD86 has higher binding capacity with the Nef protein, CD74 is non-specific peptide and is a peptide negative control for Nef binding. The coated plates are incubated overnight at 4° C. The plates were washed 5 times with PBS (0.02% Tween-20) before every step. After washing, the wells were blocked with 5% blotto and incubated at RT with shaking (650 rpm). The plates were washed 5 times with PBS (0.02%-tween20) after every step. Primary antibody mouse anti-Nef raised in rabbit dissolved in 2.5% blotto (1:1000) were added and incubated on shaker for 1 hour at RT. Plate was washed and 100 μL anti rabbit raised in donkey dissolved in 2.5% blotto (1:11000) was added and incubated at RT for 45 min. Plate was washed with PBS and TMB substrate solution was added and incubated in dark for 5-10 min. The reaction was stopped with 1N H₂SO₄ and the absorbance was measured at 450 nm. Positive control (Nef) and Negative control (No peptide with Nef) were analysed for highest binding of Nef with peptide. The absorbance of the plates was measured at 450 nm.

A throughput screen was performed to pull out the actives. Standard quality control metrics like Z′ were used to qualify plates and Z′ scores were used to identify actives. The IC₅₀ value of the compounds were further determined to evaluate the efficacy of the test compounds.

The IC₅₀ values of the compounds have been illustrated in Table 1. Eighteen compounds were found to show HIV inhibiting activity and increasing the expression of CD80 and CD86 proteins. Actives predominantly belong to compounds from Formula I, II and III with IC₅₀s in nano or subnanomolar ranges were selected. Selected compounds were then screened in the cell-based assays systems.

TABLE 1
pIC₅₀ values of representative actives from Primary Screen

| | | | CD80 pIC50 | CD86 pIC50 |
|---|---|---|---|---|
| SI No | Formula | Compound ID | value | value |
| 1 | Formula I | I₁a | 11.63 | |
| 2 | | I₂a | 15.04 | |
| 3 | | I₂b | 15.52 | |
| 4 | | I₃a | 10.96 | |
| 5 | | I₃b | 9.89 | 8.79 |
| 6 | Formula II | II₁ | 10.04 | |
| 7 | | II₃ | | 10.9 |
| 8 | | II₄ | 10.56 | 9.56 |
| 9 | | II₅ | 11.56 | |
| 10 | | II₆ | 12.46 | |
| 11 | | II₇ | 10.06 | |
| 12 | | II₂a | 14.7 | |
| 13 | | II₂c | 12.71 | |
| 14 | | II₂d | 7.98 | 6.29 |
| 15 | Formula III | III₁e | 8.63 | |
| 16 | | III₁a | 17.03 | |
| 17 | | III₂a | 11.35 | |
| 18 | | III₂b | 8.95 | |
| 19 | | III₂c | 8.82 | |

Table 1 illustrates the efficacy of individual compounds in inhibiting the Nef mediated downregulation of CD80 and CD86 proteins. Out of the total 19 compounds of Formula I, II, and III, the compounds which were found effective in inhibiting Nef-CD80 binding, in terms of higher pIC₅₀ values (IC₅₀s in nano or subnanomolar ranges), were compounds I₁a (pIC₅₀=11.63), I₂a (pIC₅₀=15.04), I₂b (pIC₅₀=15.52), I₃a (pIC₅₀=10.96), I₃b (pIC₅₀=9.89) of Formula I, compounds II₁ (pIC₅₀=10.04), II₄ (pIC₅₀=10.56), II₆ (pIC₅₀=12.46), II₅ (pIC₅₀=11.52), II₂c (pIC₅₀=12.71), II₇ (pIC₅₀=10.06), II₂d (pIC₅₀=7.98), II₂a (pIC₅₀=14.70) of Formula II, and III₂b (pIC₅₀=8.95), III₂a (pIC₅₀=11.35), III₁e (pIC₅₀=8.63), III₁a (pIC₅₀=17.03), III₂c (pIC₅₀=8.82) of Formula III respectively. Some of the effective compounds in terms of higher pIC₅₀ values were I₂a (pIC₅₀=15.04), I₂b (pIC₅₀=15.52), III₁a (pIC₅₀=17.03), and II₂a (pIC₅₀=14.70) respectively. On the other hand, the compounds which were found effective in inhibiting Nef-CD86 binding were I₃b (pIC₅₀=8.79) of Formula I and II₄ (pIC₅₀=9.56), II₃ (pIC₅₀=10.90) and II₂d (pIC₅₀=6.29) of Formula II. Thus, the compound which were active in inhibiting interactions of Nef protein with both CD80 and CD86 proteins were I₂b, II₄ and II₂d respectively.

### Example 2

### Cell-Based Screen: Phenotypic Screen for Downregulation

The schematic of the cell-based assay is as shown in the FIG. 2. CD80 and CD86 are two major co-stimulatory cell surface proteins that are present on antigen presenting cells (APCs) and provide a co-stimulatory signal necessary for activation and survival of the naïve T cells. Both these proteins get down regulated during HIV infection via a Nef mediated pathway. It has been speculated that Nef ability to reduce CD80 and CD86 surface expression in infected cells can prevent the activation of naive T cells, necessary for an efficient recognition and elimination of the target cells. FIG. 2 illustrates that in the presence of the compounds of Formula I, Formula II or Formula III, the binding of Nef with CD80 and CD86 is inhibited thus increasing their expression. The increased expression would activate naïve T cells and aid in eliminating the infection.

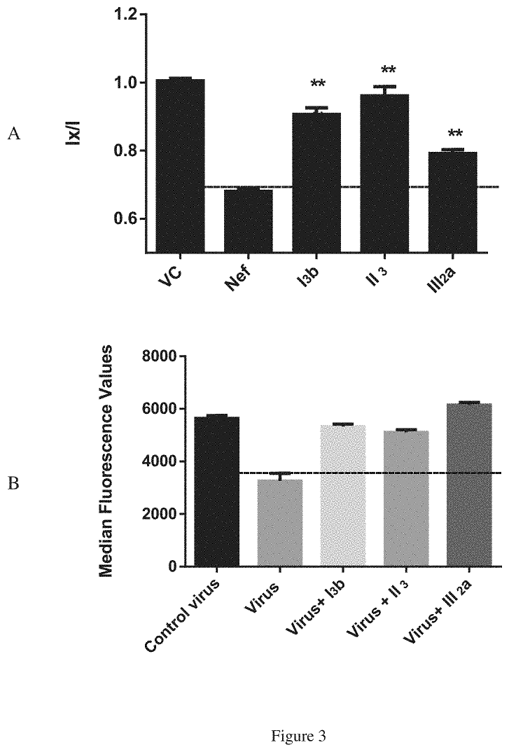

Briefly monocyte/B cell lines were either delivered with Nef protein or exposed to replication deficient retrovirus and the levels of surface CD80/86 was evaluated by flow cytometry with/without compound treatment. The compounds were qualified based on the ability to reverse the down regulation of CD80/86 caused by the Nef protein. Similar quality checks were used in the secondary screen as in primary screen. Results from representative compounds were tested in the phenotypic assay for downregulation is presented in the FIG. 3. Briefly, B/monocyte cells were preexposed to 100 μM of the 4 compounds for 2 hrs and then infected with non-replicative retrovirus containing NEF. The internalization of surface receptors were measured by flow cytometry after optimal infection (48-72 hrs). Representative compounds from each scaffold (I₃b, II₃ and III₂a) showed reversal of Nef mediated internalization of CD80/86.

### Example 3

### Efficacy Assay System to Test T Cell Activation:

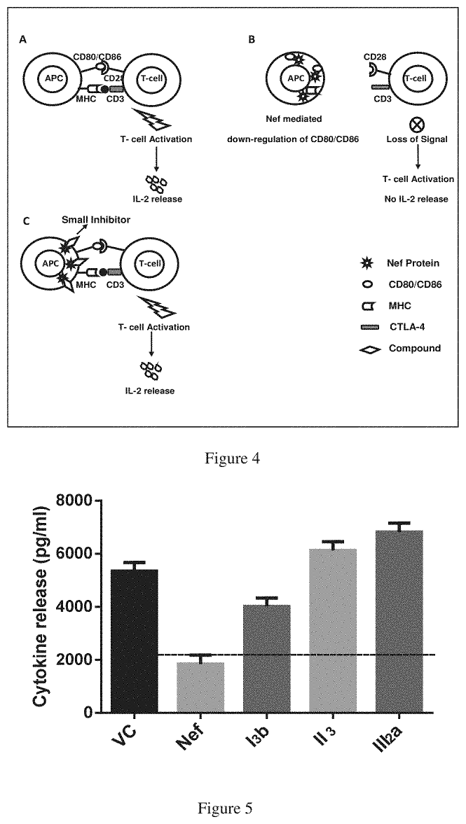

The appropriate cell system was designed to mimic the biology of the said interactions. The schematic representation of the cell-based T cell activation assay is as shown in FIG. 4. FIG. 4(A) represents the principle behind the activation of naïve T cells by the antigen presenting cells (APC), including dendritic cells, B cells, monocytes, macrophages. The activation is based on CD80/86 co-stimulation along with CD3 activation by major histocompatibility complex (MHC) as evidenced by an increase in IL-2 release from the activated T cells.

FIG. 4(B) is a schematic representation that displays the addition of Nef in the APC causes down regulation of the CD80/86 and hence loss of T cell activation, which leads to a reduction in IL-2 release.

FIG. 4(C) displays the rescue of internalization of CD80/86 back to the surface of the APC by means of inhibitors to the Nef protein delivered to the APC cell line, which restores CD80/86 co-stimulation and hence results in IL-2 release from the T cells.

Representative compounds from the Formula I, II and III were tested in the functional T cell assay and the results are as presented in FIG. 5. B-cell line was pre-exposed to 100 μM of the 4 compounds for 1 hr. The cells were washed and then Nef was introduced via protein delivery agent. The cells were washed again and co-cultured with T cell line. The IL-2 release (as an indicator of activation) was measured by ELISA after 3 hrs. Addition of Nef reduced the T cell activation by more than 50%. Addition of compounds (I₃b, II₃ and III₂a) reversed the Nef mediated inactivation.

### Advantage

The method of treatment of HIV infection by targeting Nef-CD80/86 interactions has a very distinct and unique mechanism of action: prevention of immune evasion of infected cells. None of the potential or existing therapy has targeted the viral strategy of immune evasion and the present disclosure presents a new class of drugs. Also, targeting host-virus interface is also less amenable to drug resistance than pure viral targets. This, hence, would provide hope to class of people where existing retroviral treatments have failed and also a profound impact on pre-exposure prophylaxis to the hot-spot population.

1. A method for treating HIV subtypes causing disease in a subject comprising:
administering to a HIV infected subject a therapeutic dose of a compound selected from a group consisting of Formula I, 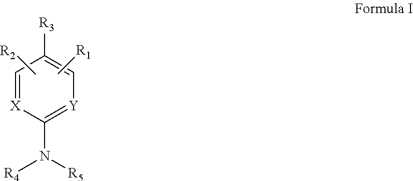
or its stereoisomers, pharmaceutically acceptable salts, polymorphs, solvates and hydrates thereof, wherein
R₁ and R₂ are independently selected from the group consisting of hydrogen, C₆₋₁₀ aryl, C₁₋₁₀ alkyl, C₂₋₁₀ alkenyl, C₂₋₁₀ alkynyl, and amino, wherein C₁₋₁₀ alkyl, C₂₋₁₀ alkenyl, C₂₋₁₀ alkynyl, and C₆₋₁₀ aryl are optionally substituted with one to four substituents independently selected from hydroxyl, halogen, C₁₋₁₀ alkyl, C₁₋₁₀ alkoxy or C₂₋₁₀ heterocyclyl;
R₃ is C₆₋₁₀ aryl, wherein C₆₋₁₀ aryl is optionally substituted with one to four substituents independently selected from C₁₋₁₀ alkoxy, nitro, halogen or C₁₋₁₀ alkyl, wherein C₁₋₁₀ alkyl, and C₁₋₁₀ alkoxy is optionally substituted with one to four substituents selected from halogen, hydrogen, hydroxyl, C₁₋₁₀ alkyl, C₁₋₁₀ alkoxy or C₂₋₁₀ heterocyclyl;
R₄ and R₅ are independently selected from the group consisting of hydrogen, C₁₋₁₀ alkyl, and —N═CHC₆ aryl, wherein N═CHC₆ aryl is further substituted with one to four halogen; or R₄ and R₅ are joined to form a C₂₋₁₀ heterocyclyl or a C₂₋₁₀ heteroaryl, wherein C₂₋₁₀ heterocyclyl or C₂₋₁₀ heteroaryl is optionally substituted with one to four substituents independently selected from C₁₋₁₀ alkyl; and
X and Y are N; 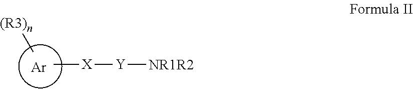
or its stereoisomers, pharmaceutically acceptable salts, polymorphs, solvates and hydrates thereof, wherein,
R₁, and R₂ are independently selected from the group consisting of hydrogen, C₁₋₁₀ alkyl, hydroxy, thio, amino, C₂₋₁₀ heterocyclyl, C₅₋₁₀ aryl, C₂₋₁₀ heteroaryl, acyl, amido, imido, sulfinyl, sulfonyl, carboxaldehyde, cyano, isocyano, azido, hydrazino, nitro, halo; or R₁ and R₂ are joined to form an optionally substituted monocyclic or a bicyclic ring, wherein C₁₋₁₀ alkyl, C₂₋₁₀ heterocyclyl, C₅₋₁₀ aryl, C₂₋₁₀ heteroaryl or monocyclic or a bicyclic ring are optionally substituted with one four substituents independently selected from hydroxy, C₁₋₁₀ alkyl, C₅₋₁₀ aryl, C₁₋₁₀ alkoxy, halogen, haloalkyl, perhaloalkyl, cyano, thio, and —CH₂-Ph-NH—COO-Ph-Me;
X is O;
Y is C═O;
Ar is selected from C₁₋₁₀ alkyl, C₅₋₁₀ aryl or C₂₋₁₀ heteroaryl,
R₃ is absent or is selected from hydroxy, C₁₋₁₀ alkyl, C₅₋₁₀ aryl, C₂₋₁₀ heteroaryl, C₂₋₁₀ heterocyclyl, C₁₋₁₀ alkoxy, halogen, haloalkyl, perhaloalkyl, nitro, cyano, CH₂PhNHCOOPh-Me, NHCONHPh-(Cl)₂ or CONHNHSO₂Ph-Me; and
n is selected from 0-5; and 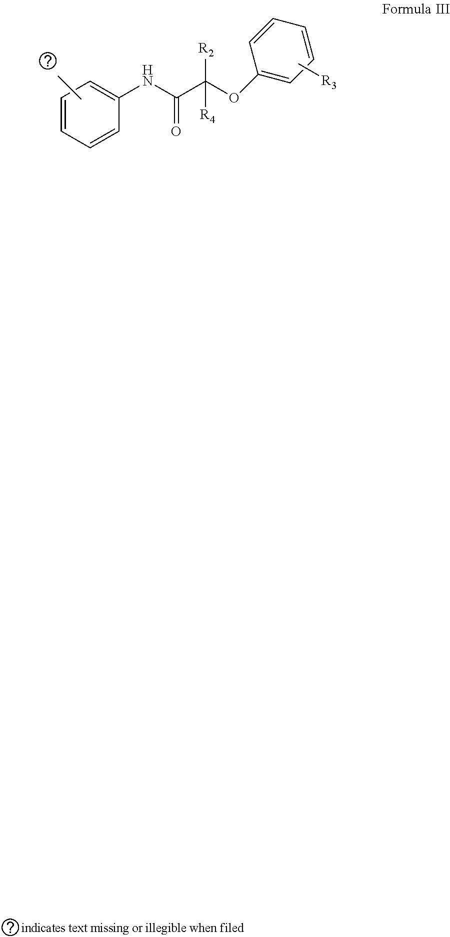
or its stereoisomers, pharmaceutically acceptable salts, polymorphs, solvates and hydrates thereof, wherein,
R₁ is independently selected from the group consisting of OH, COOH, and COORa, wherein Ra is C₁₋₁₀ alkyl;
R₂ and R₄ is independently selected from hydrogen or C₁₋₁₀ alkyl;
R₃ is selected from C₁₋₁₀ alkyl or halogen; and
n is selected from 1 to 5.

2. A method for preventing HIV subtypes causing disease in a subject in need thereof comprising: administering to a HIV high risk subject a relevant dose of a compound selected from a group consisting of 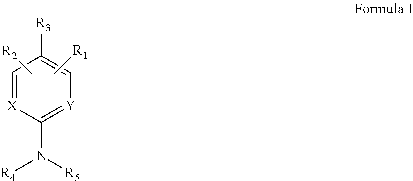
or its stereoisomers, pharmaceutically acceptable salts, polymorphs, solvates and hydrates thereof, wherein
R₁ and R₂ are independently selected from the group consisting of hydrogen, C₆₋₁₀ aryl, C₁₋₁₀ alkyl, C₂₋₁₀ alkenyl, C₂₋₁₀ alkynyl, and amino, wherein C₁₋₁₀ alkyl, C₂₋₁₀ alkenyl, C₂₋₁₀ alkynyl, and C₆₋₁₀ aryl are optionally substituted with one to four substituents independently selected from hydroxyl, halogen, C₁₋₁₀ alkyl, C₁₋₁₀ alkoxy or C₂₋₁₀ heterocyclyl;
R₃ is C₆₋₁₀ aryl, wherein C₆₋₁₀ aryl is optionally substituted with one to four substituents independently selected from C₁₋₁₀ alkoxy, nitro or C₁₋₁₀ alkyl, wherein C₁₋₁₀ alkyl, and C₁₋₁₀ alkoxy is optionally substituted with one to four substituents selected from halogen; hydrogen, hydroxyl, C₁₋₁₀ alkyl, C₁₋₁₀ alkoxy or C₂₋₁₀ heterocyclyl;
R₄ and R₅ are independently selected from the group consisting of hydrogen, C₁₋₁₀ alkyl, and —N═CHC₆ aryl, wherein N═CHC₆ aryl is further substituted with one to four halogen; or R₄ and R₅ are joined to form a C₂₋₁₀ heterocyclyl or a C₂₋₁₀ heteroaryl, wherein C₂₋₁₀ heterocyclyl or C₂₋₁₀ heteroaryl is optionally substituted with one to four substituents independently selected from C₁₋₁₀ alkyl; and
X and Y are N; 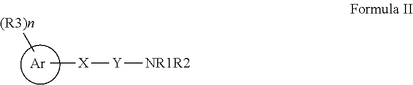
or its stereoisomers, pharmaceutically acceptable salts, polymorphs, solvates and hydrates thereof, wherein,
R₁, and R₂ are independently selected from the group consisting of hydrogen, C₁₋₁₀ alkyl, hydroxy, thio, amino, C₂₋₁₀ heterocyclyl, C₅₋₁₀ aryl, C₂₋₁₀ heteroaryl, acyl, amido, imido, sulfinyl, sulfonyl, carboxaldehyde, cyano, isocyano, azido, hydrazino, nitro, halo; or R₁ and R₂ are joined to form an optionally substituted monocyclic or a bicyclic ring, wherein C₁₋₁₀ alkyl, C₂₋₁₀ heterocyclyl, C₅₋₁₀ aryl, C₂₋₁₀ heteroaryl or monocyclic or a bicyclic ring are optionally substituted with one four substituents independently selected from hydroxy, C₁₋₁₀ alkyl, C₅₋₁₀ aryl, C₁₋₁₀ alkoxy, halogen, haloalkyl, perhaloalkyl, cyano, thio, and —CH₂-Ph-NH—COO-Ph-Me;
X is O;
Y is C═O;
Ar is selected from C₁₋₁₀ alkyl, C₅₋₁₀ aryl or C₂₋₁₀ heteroaryl,
R₃ is absent or is selected from hydroxy, C₁₋₁₀ alkyl, C₅₋₁₀ aryl, C₂₋₁₀ heteroaryl, C₂₋₁₀ heterocyclyl, C₁₋₁₀ alkoxy, halogen, haloalkyl, perhaloalkyl, nitro, cyano, CH₂PhNHCOOPh-Me, NHCONHPh-(Cl)₂ or CONHNHSO₂Ph-Me; and
n is selected from 0-5; and 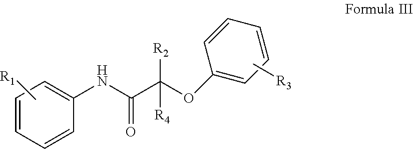
or its stereoisomers, pharmaceutically acceptable salts, polymorphs, solvates and hydrates thereof, wherein,
R₁ is independently selected from the group consisting of OH, COOH, and COORa, wherein Ra is C₁₋₁₀ alkyl;
R₂ and R₄ is independently selected from hydrogen or C₁₋₁₀ alkyl;
R₃ is selected from C₁₋₁₀ alkyl or halogen; and
n is selected from 1 to 5.

3. The method according to claim 1, wherein compound of Formula I is selected from a group consisting of:

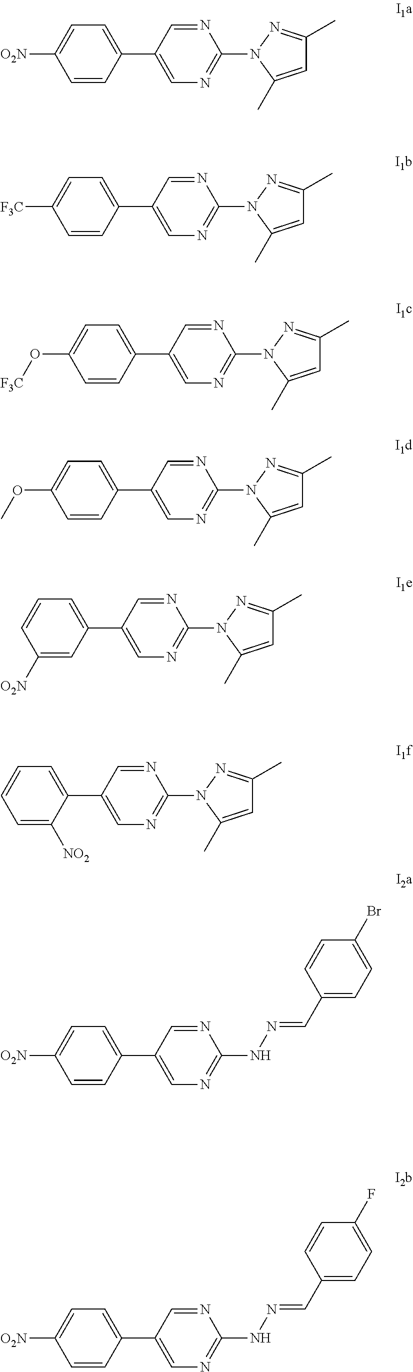

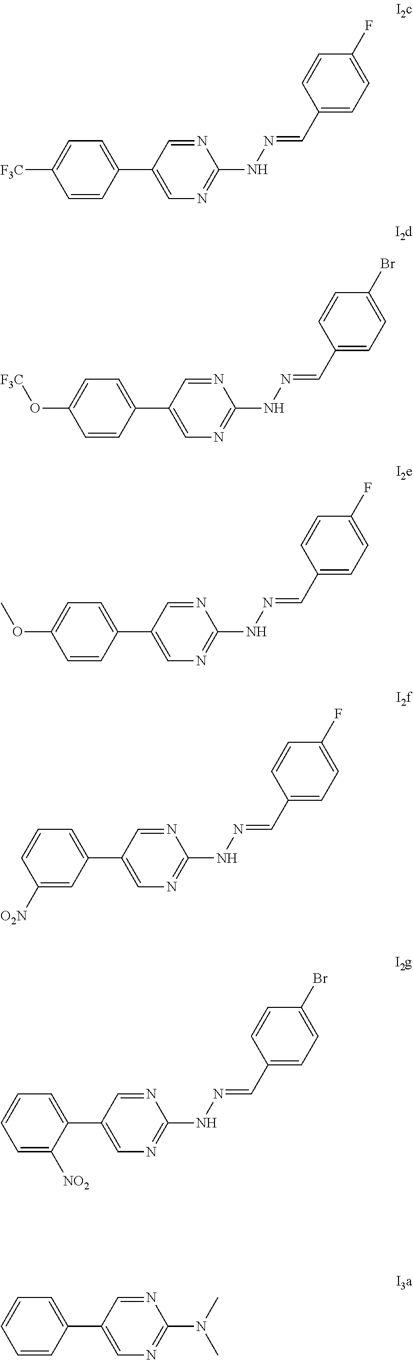 [SMILES: Fc1ccc(/C=N/Nc2ncc(-c3ccc(C(F)(F)F)cc3)cn2)cc1 ; FC(F)(F)Oc1ccc(-c2cnc(N/N=C/c3ccc(Br)cc3)nc2)cc1 ; COc1ccc(-c2cnc(N/N=C/c3ccc(F)cc3)nc2)cc1 ; O=[N+]([O-])c1cccc(-c2cnc(N/N=C/c3ccc(F)cc3)nc2)c1 ; O=[N+]([O-])c1ccccc1-c1cnc(N/N=C/c2ccc(Br)cc2)nc1 ; CN(C)c1ncc(-c2ccccc2)cn1]

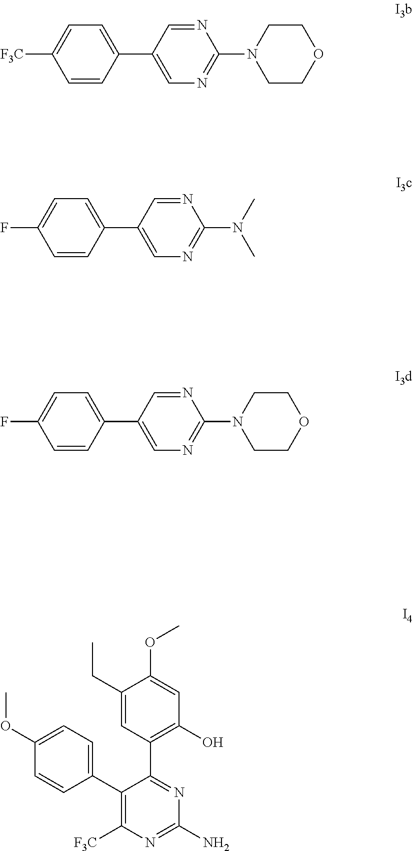 [SMILES: FC(F)(F)c1ccc(-c2cnc(N3CCOCC3)nc2)cc1 ; CN(C)c1ncc(-c2ccc(F)cc2)cn1 ; Fc1ccc(-c2cnc(N3CCOCC3)nc2)cc1 ; CCc1cc(-c2nc(N)nc(C(F)(F)F)c2-c2ccc(OC)cc2)c(O)cc1OC]

4. The method according to claim 1, wherein compound of Formula I is selected from a group consisting of:

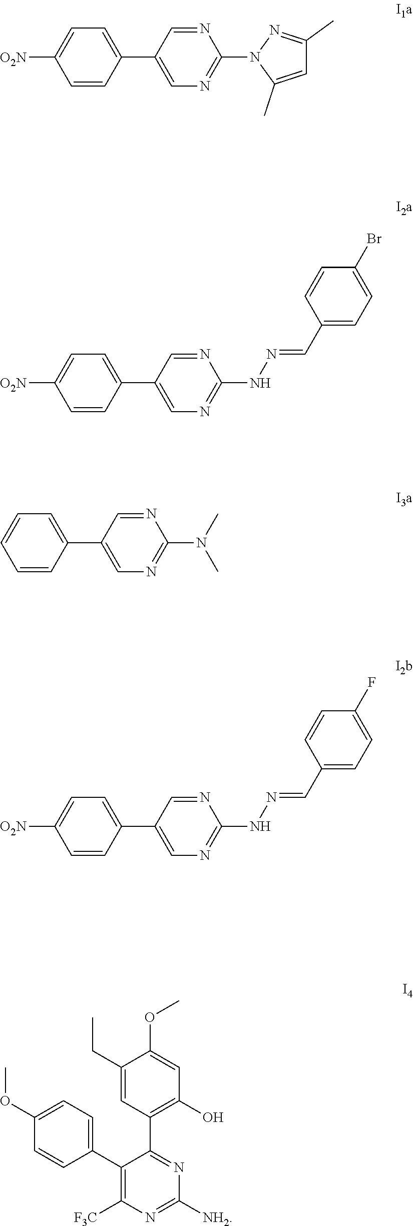 [SMILES: Cc1cc(C)n(-c2ncc(-c3ccc([N+](=O)[O-])cc3)cn2)n1 ; O=[N+]([O-])c1ccc(-c2cnc(N/N=C/c3ccc(Br)cc3)nc2)cc1 ; CN(C)c1ncc(-c2ccccc2)cn1 ; O=[N+]([O-])c1ccc(-c2cnc(N/N=C/c3ccc(F)cc3)nc2)cc1 ; CCc1cc(-c2nc(N)nc(C(F)(F)F)c2-c2ccc(OC)cc2)c(O)cc1OC]

5. The method according to claim 1, wherein compound of Formula II is selected from a group consisting of:

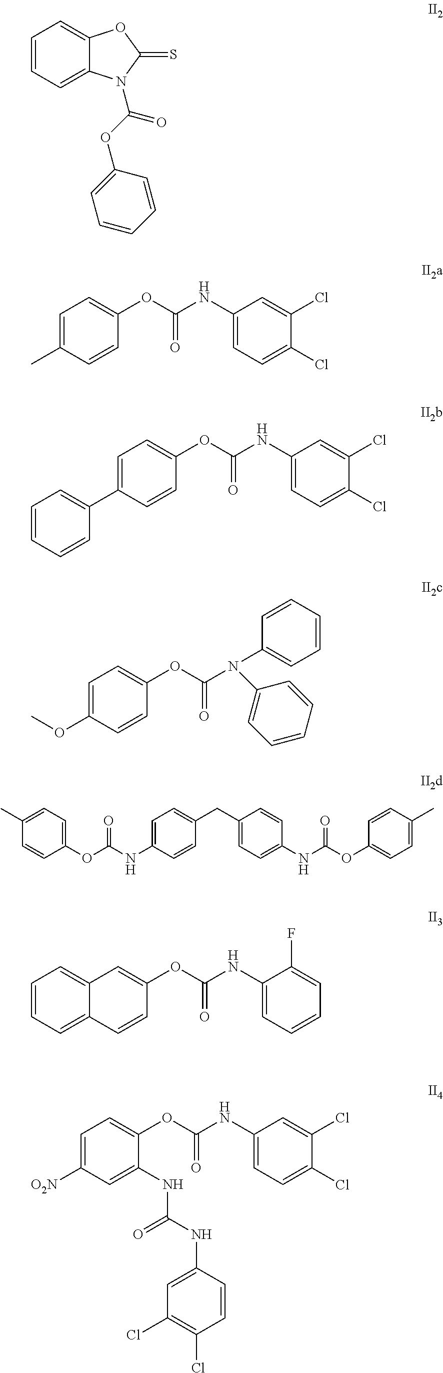

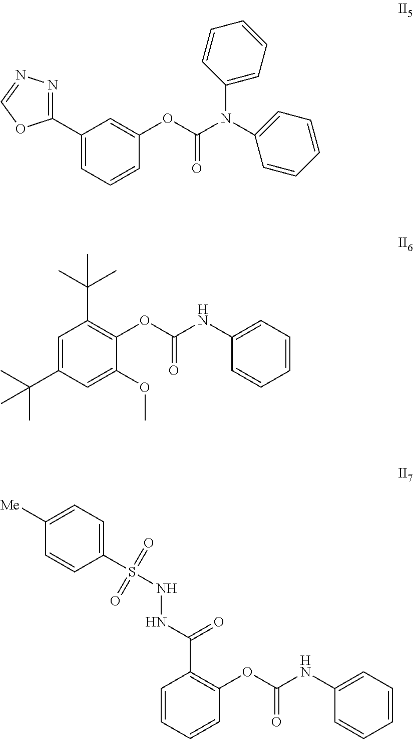 [SMILES: O=C(Oc1cccc(-c2nnco2)c1)N(c1ccccc1)c1ccccc1 ; COc1cc(C(C)(C)C)cc(C(C)(C)C)c1OC(=O)Nc1ccccc1 ; Cc1ccc(S(=O)(=O)NNC(=O)c2ccccc2OC(=O)Nc2ccccc2)cc1]

6. The method according to claim 1, wherein compound of Formula III is selected from a group consisting of:

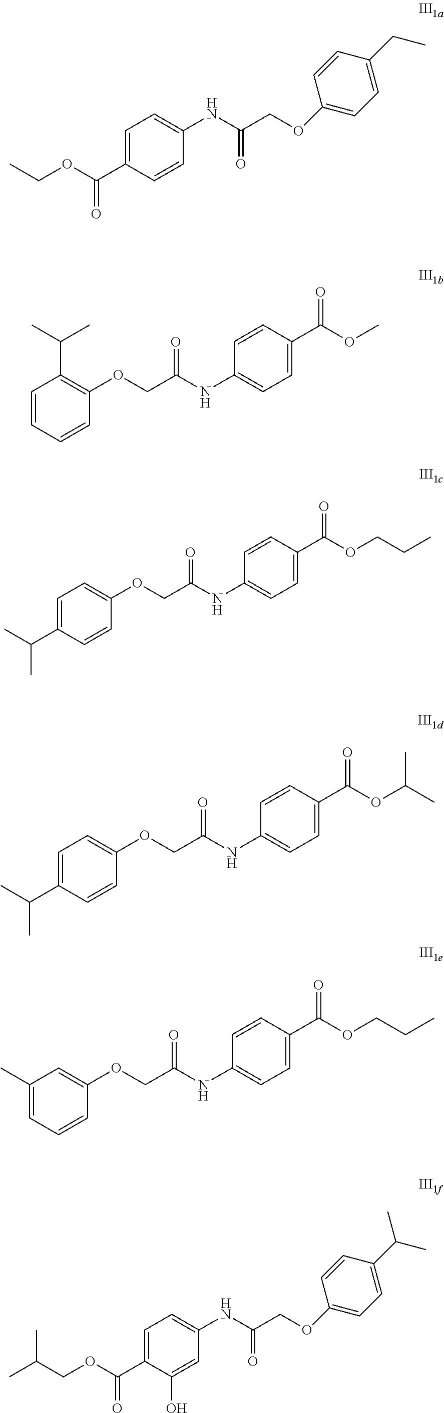

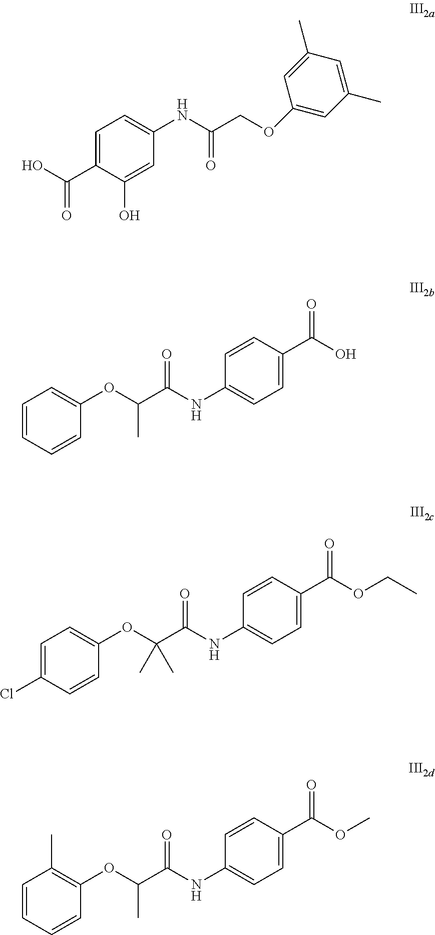 [SMILES: Cc1cc(C)cc(OCC(=O)Nc2ccc(C(=O)O)c(O)c2)c1 ; CC(Oc1ccccc1)C(=O)Nc1ccc(C(=O)O)cc1 ; CCOC(=O)c1ccc(NC(=O)C(C)(C)Oc2ccc(Cl)cc2)cc1 ; COC(=O)c1ccc(NC(=O)C(C)Oc2ccccc2C)cc1]

7. A method of treating or preventing HIV infection in a subject by immune evasion mediated by internalization of CD80/86 receptors by its Nef protein comprising:
administering to a subject a relevant dose of a compound selected from a group consisting of 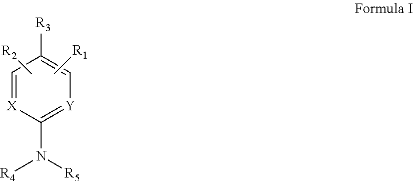
or its stereoisomers, pharmaceutically acceptable salts, polymorphs, solvates and hydrates thereof, wherein
R₁ and R₂ are independently selected from the group consisting of hydrogen, C₆₋₁₀ aryl, C₁₋₁₀ alkyl, C₂₋₁₀ alkenyl, C₂₋₁₀ alkynyl, and amino, wherein C₁₋₁₀ alkyl, C₂₋₁₀ alkenyl, C₂₋₁₀ alkynyl, and C₆₋₁₀ aryl are optionally substituted with one to four substituents independently selected from hydroxyl, halogen, C₁₋₁₀ alkyl, C₁₋₁₀ alkoxy or C₂₋₁₀ heterocyclyl;
R₃ is C₆₋₁₀ aryl, wherein C₆₋₁₀ aryl is optionally substituted with one to four substituents independently selected from C₁₋₁₀ alkoxy, nitro or C₁₋₁₀ alkyl, wherein C₁₋₁₀ alkyl, and C₁₋₁₀ alkoxy is optionally substituted with one to four substituents selected from halogen; hydrogen, hydroxyl, C₁₋₁₀ alkyl, C₁₋₁₀ alkoxy or C₂₋₁₀ heterocyclyl;
R₄ and R₅ are independently selected from the group consisting of hydrogen, C₁₋₁₀ alkyl, and —N═CHC₆ aryl, wherein N═CHC₆ aryl is further substituted with one to four halogen; or R₄ and R₅ are joined to form a C₂₋₁₀ heterocyclyl or a C₂₋₁₀ heteroaryl, wherein C₂₋₁₀ heterocyclyl or C₂₋₁₀ heteroaryl is optionally substituted with one to four substituents independently selected from C₁₋₁₀ alkyl; and
X and Y are N; 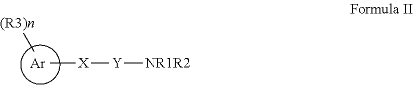
or its stereoisomers, pharmaceutically acceptable salts, polymorphs, solvates and hydrates thereof, wherein,
R₁, and R₂ are independently selected from the group consisting of hydrogen, C₁₋₁₀ alkyl, hydroxy, thio, amino, C₂₋₁₀ heterocyclyl, C₅₋₁₀ aryl, C₂₋₁₀ heteroaryl, acyl, amido, imido, sulfinyl, sulfonyl, carboxaldehyde, cyano, isocyano, azido, hydrazino, nitro, halo; or R₁ and R₂ are joined to form an optionally substituted monocyclic or a bicyclic ring, wherein C₁₋₁₀ alkyl, C₂₋₁₀ heterocyclyl, C₅₋₁₀ aryl, C₂₋₁₀ heteroaryl or monocyclic or a bicyclic ring are optionally substituted with one four substituents independently selected from hydroxy, C₁₋₁₀ alkyl, C₅₋₁₀ aryl, C₁₋₁₀ alkoxy, halogen, haloalkyl, perhaloalkyl, cyano, thio, and —CH₂-Ph-NH—COO-Ph-Me;
X is O;
Y is C═O;
Ar is selected from C₁₋₁₀ alkyl, C₅₋₁₀ aryl or C₂₋₁₀ heteroaryl,
R₃ is absent or is selected from hydroxy, C₁₋₁₀ alkyl, C₅₋₁₀ aryl, C₂₋₁₀ heteroaryl, C₂₋₁₀ heterocyclyl, C₁₋₁₀ alkoxy, halogen, haloalkyl, perhaloalkyl, nitro, cyano, CH₂PhNHCOOPh-Me, NHCONHPh-(Cl)₂ or CONHNHSO₂Ph-Me; and
n is selected from 0-5; and 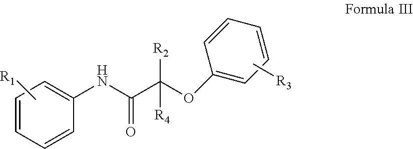
or its stereoisomers, pharmaceutically acceptable salts, polymorphs, solvates and hydrates thereof, wherein,
R₁ is independently selected from the group consisting of OH, COOH, and COORa, wherein Ra is C₁₋₁₀ alkyl;
R₂ and R₄ is independently selected from hydrogen or C₁₋₁₀ alkyl;
R₃ is selected from C₁₋₁₀ alkyl or halogen; and
n is selected from 1 to 5.

8. The method as claimed in claim 7, wherein the compounds causes restoration of immune signaling via T cell activation through inhibiting Nef-CD80/86 interactions.

9. (canceled)

10. The method according to claim 2, wherein compound of Formula I is selected from a group consisting of:

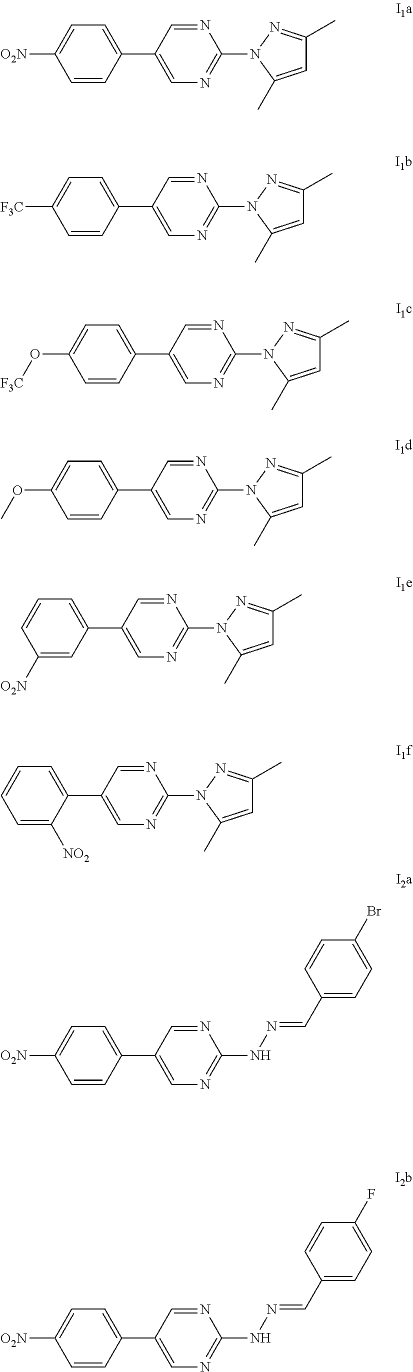

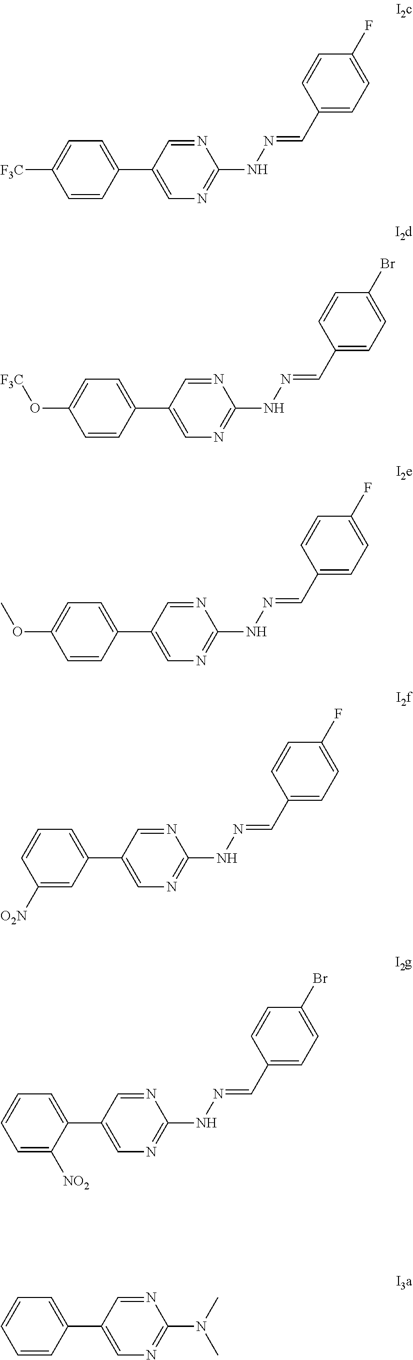 [SMILES: Fc1ccc(/C=N/Nc2ncc(-c3ccc(C(F)(F)F)cc3)cn2)cc1 ; FC(F)(F)Oc1ccc(-c2cnc(N/N=C/c3ccc(Br)cc3)nc2)cc1 ; COc1ccc(-c2cnc(N/N=C/c3ccc(F)cc3)nc2)cc1 ; O=[N+]([O-])c1cccc(-c2cnc(N/N=C/c3ccc(F)cc3)nc2)c1 ; O=[N+]([O-])c1ccccc1-c1cnc(N/N=C/c2ccc(Br)cc2)nc1 ; CN(C)c1ncc(-c2ccccc2)cn1]

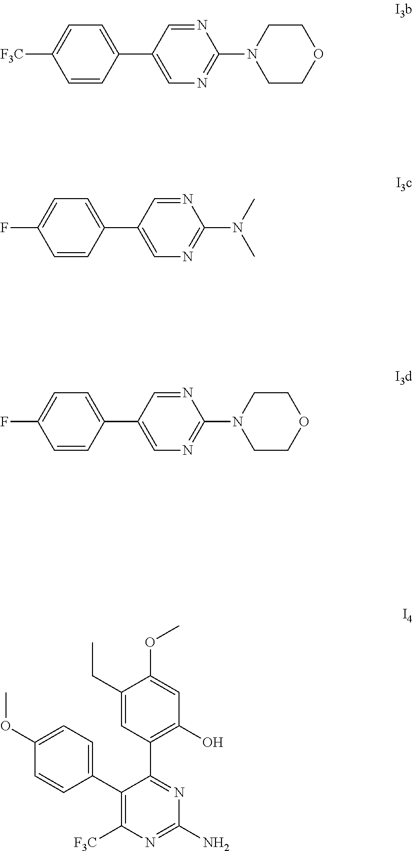 [SMILES: FC(F)(F)c1ccc(-c2cnc(N3CCOCC3)nc2)cc1 ; CN(C)c1ncc(-c2ccc(F)cc2)cn1 ; Fc1ccc(-c2cnc(N3CCOCC3)nc2)cc1 ; CCc1cc(-c2nc(N)nc(C(F)(F)F)c2-c2ccc(OC)cc2)c(O)cc1OC]

11. The method according to claim 2, wherein compound of Formula I is selected from a group consisting of:

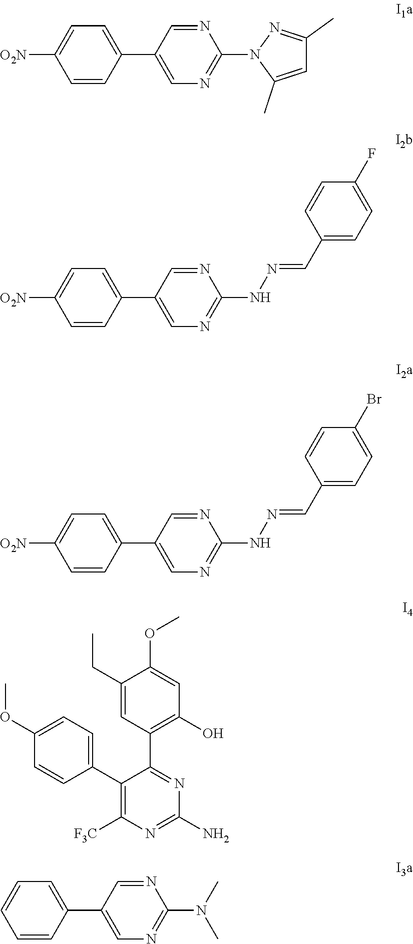

12. The method according to claim 2, wherein compound of Formula II is selected from a group consisting of:

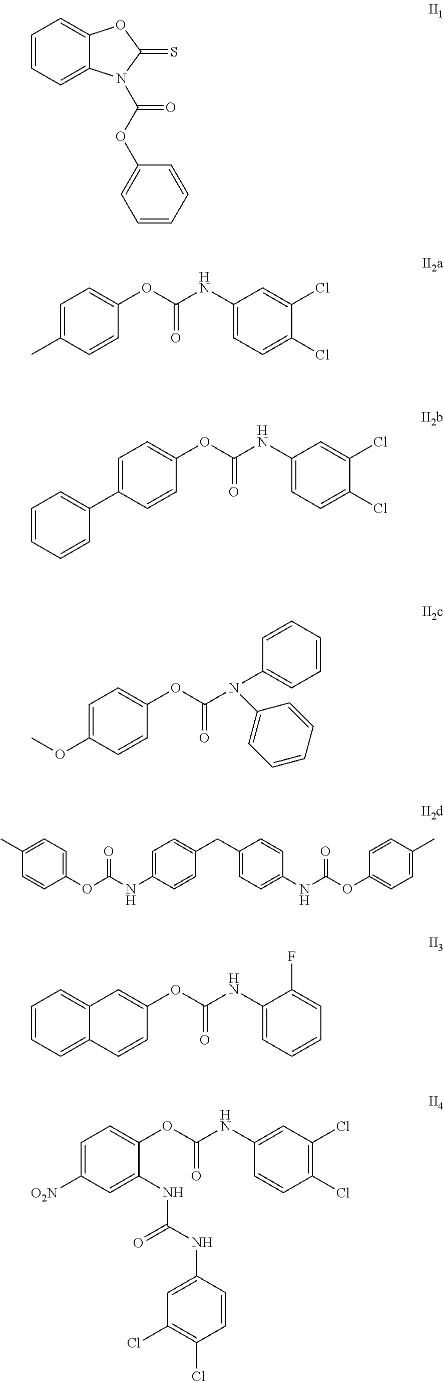

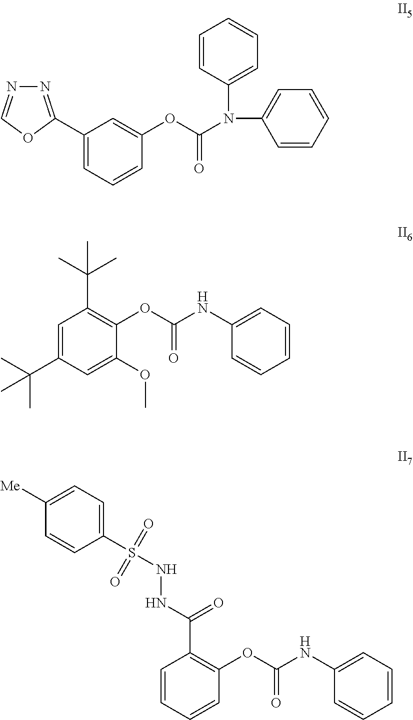 [SMILES: O=C(Oc1cccc(-c2nnco2)c1)N(c1ccccc1)c1ccccc1 ; COc1cc(C(C)(C)C)cc(C(C)(C)C)c1OC(=O)Nc1ccccc1 ; Cc1ccc(S(=O)(=O)NNC(=O)c2ccccc2OC(=O)Nc2ccccc2)cc1]

13. The method according to claim 2, wherein compound of Formula III is selected from a group consisting of: 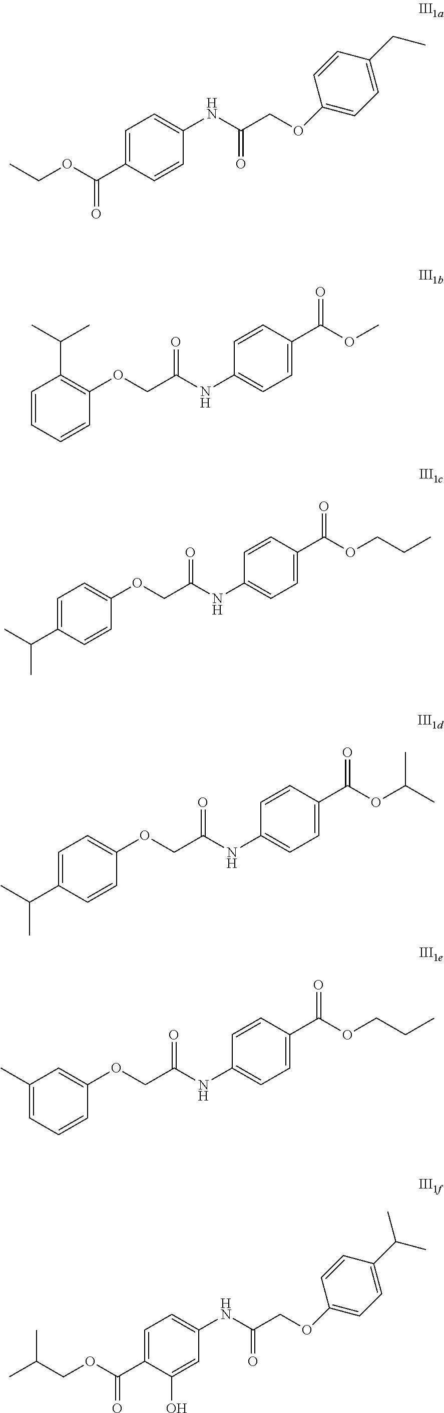 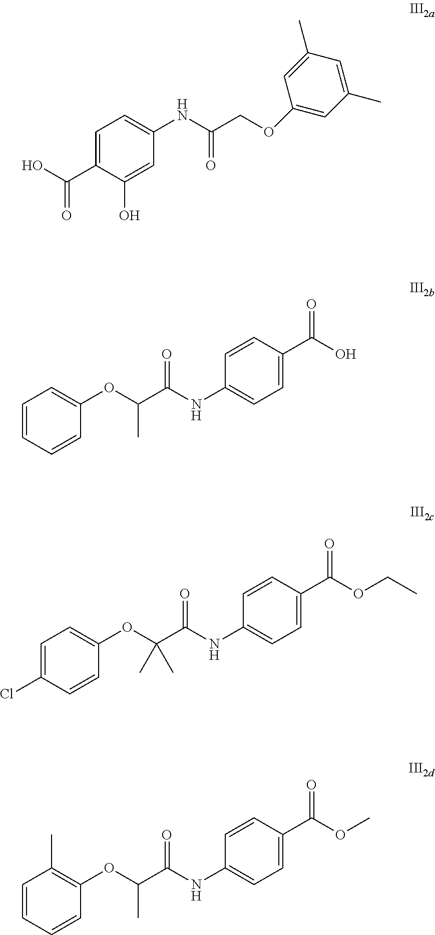 [SMILES: Cc1cc(C)cc(OCC(=O)Nc2ccc(C(=O)O)c(O)c2)c1 ; CC(Oc1ccccc1)C(=O)Nc1ccc(C(=O)O)cc1 ; CCOC(=O)c1ccc(NC(=O)C(C)(C)Oc2ccc(Cl)cc2)cc1 ; COC(=O)c1ccc(NC(=O)C(C)Oc2ccccc2C)cc1]
